(19) United States
(12) Patent Application Publication
(10) Pub. No.: US 2002/0077276 A1
(43) Pub. Date: Jun. 20, 2002

(54) Compositions and methods for treating hemorrhagic virus infections and other disorders

(76) Inventors: Terry M. Fredeking, Bedford, TX (US); George M. Ignatyev, Koltsovo (RU)

Correspondence Address: HELLER EHRMAN WHITE & MCAULIFFE LLP, 4250 EXECUTIVE SQ, 7TH FLOOR, LA JOLLA, CA, 92037, US

(21) Appl. No.: 09/840,707

(22) Filed: Apr. 23, 2001

Related U.S. Application Data
(62) Division of application No. 09/562,979, filed on Apr. 27, 2000.
(60) Provisional application No. 60/198,210, filed on Apr. 27, 1999.

(51) Int. Cl. A01N 37/18; A61K 38/00

(52) U.S. Cl. 514/2

(57) ABSTRACT

Cytokine-receptor and cytokine antagonist-enriched blood-dervided compositions and methods of preparing and using the compositions are provided. Also provided are compositions and methods for the treatment or prevention of disorders, especially acute inflammatory disorders involving pathological responses of the immune system, such as viral hemorrhagic diseases, sepsis, rheumatoid arthritis and other autoimmune disorders, acute cardiovascular events, flare-ups and acute phases of multiple sclerosis, wasting disorders and other disorders involving deleterious expression of cytokines and other factors, including tumor necrosis factor (TNF) and interleukin-1 (IL-1) are provided.

### RELATED APPLICATIONS

This application is a divisional of U.S. application Ser. No. 09/562,979, filed Apr. 27, 2000, by Terry M. Fredeking and George M. lgnatyev, entitled “COMPOSITIONS AND METHODS FOR TREATING HEMORRHAGIC VIRUS INFECTIONS AND OTHER DISORDERS”. Benefit of priority under 35 U.S.C. §119(e) to U.S. provisional application Ser. No. 60/198,210, which was filed as U.S. application Ser. No. 09/301,274, filed Apr. 27, 1999, and converted to a provisional on Apr. 27, 2000, by Terry M. Fredeking and George M. Ignatyev, entitled “COMPOSITIONS AND METHODS FOR TREATING HEMORRHAGIC VIRUS INFECTIONS AND OTHER DISORDERS”, is claimed herein.

The subject matter of each of U.S. application Ser. Nos. 09/562,979 and 09/301,274 is incorporated by reference in its entirety.

### FIELD OF INVENTION

The present invention relates to compositions and methods for treating and/or preventing in mammals, particularly humans, acute inflammatory responses and diseases. More particularly, compositions and combinations of compositions and methods for the treatment of disorders, especially acute inflammatory disorders, involving pathological responses of the immune system are provided. Hence the disclosure herein provides compositions and methods for preventing and/or treating diseases, disorders and conditions that include viral hemorrhagic diseases and other acute infectious diseases, sepsis, rheumatoid arthritis and other autoimmune disorders, acute cardiovascular events, flare-ups and acute phases of multiple sclerosis, wasting disorders and other disorders involving deleterious expression of cytokines and other factors, including tumor necrosis factor (TNF) and interleukin-1 (IL-1).

### BACKGROUND OF THE INVENTION

### Diseases and Disorders Associated with or Characterized by Acute Inflammatory Responses

Responses of the immune system to pathogens and to other bodily insults are essential for survival of mammals. Inappropriate or excessive response, however, is associated with certain acute and chronic diseases. In such cases, inappropriate stimulation of various defense strategies involving inflammatory cells and the immune system produces the symptoms characteristic of the disease. The response of a mammal to infection with a hemorrhagic virus or a pathogenic strain of Escherichia coli and sepsis are exemplary of such responses. There are few, if any, effective treatments to counteract these responses.

### Interleukin-1 and Receptors Therefor

The two forms of lnterleukin-1 (IL-1α and IL-1β) are cytokines produced primarily by mononuclear phagocytes, but also by a number of other cell types including skin keratinocytes, some epithelial cells, and some cells of the central nervous system (CNS). These cytokines produce a wide variety of effects on numerous cell types, including the induction or suppression of the production of a great number of other proteins including interleukins, cytokines, tumor necrosis factors, and colony stimulating factors. IL-1α and IL-1β are thus important mediators of the inflammatory and immune responses of animals. Because of the early appearance of IL-1 during the inflammatory reaction and the immune response, and because of the variety of effects produced by IL-1α and IL-1β, these factors play a role in the production of pathological conditions resulting in chronic inflammation, septic shock, and defects in hematopoiesis. The effects of these interleukins result from the binding of these factors to two distinct cell surface receptors, IL-1R Types 1 and II. Type I receptor is an 80 kDa protein found on T cells, fibroblasts, and keratinocytes. Type II receptor is a 68 kDa protein found on B cells and polymorphonuclear leukocytes (PMNs). In general, the Type I receptor binds to IL-1α or IL-1β with approximately equal affinity and the Type II receptor binds IL-1β more strongly than IL-1α. Results indicate that only the Type I receptor is capable of transducing a signal and can produce all of the biological effects attributed to IL-1. It has been suggested that the function of the membrane-bound Type II receptor is to serve as the precursor for a soluble IL-1 binding factor that can be shed under appropriate circumstances to antagonize and modulate IL-1 activity. A naturally occurring IL-1 binding protein has been described that seems to correspond to the soluble external portion of the Type II receptor.

A different type of naturally occurring inhibitor of IL-1 activity was discovered and purified from the urine of patients with monocytic leukemia. A cDNA clone encoding this polypeptide has been isolated from monocytes and found to code for a mature 152 amino acid residue glycoprotein of 25,000 molecular weight. This molecule, known as secreted IL-1 receptor antagonist (slL-1 Ra), shows 25% amino acid homology to IL-1β and 19% homology to IL-1α. Evidence indicates that the inhibitory action of sIL-1Ra results from binding of IL-1Ra to the IL-1 receptor Type I with an affinity comparable to that of IL-1α or IL-1β (Kd -200 pM), thus competing with IL-1α or β for binding to this receptor. This binding, however, does not result in signal transduction. IL-1Ra binds to the IL-1 receptor Type II with considerably lower affinity than that shown by IL-1β.

Cells know to produce IL-1ra include monocytes, neutrophils, macrophages and fibroblasts. Cytokines known to upregulate IL-1Ra production include IL-13, IL-6, IL-4, IFN-γ, GM-CSF and TGF-β, the latter apparently by triggering IL-1 production which itself triggers IL-1ra synthesis. The amino acid sequences of IL-1ra from at least four species have been determined (human, rat, mouse and rabbit) and found to be at least 75% homologous (Cominelli etal. (1994) J. Biol. Chem. 269:6963), IL-1ra can also be synthesized as a strictly intracellular form whose production is the result of an alternative splicing of exon 1 (Butcher et al. (1994) J. Immunol. 153:701; Arend et al. (1993) Adv. Immunol. 54:167). IL-1Ra is released in vivo during experimentally-induced inflammation and as part of the natural course of many diseases. Administered experimentally, IL-1Ra has been demonstrated to block IL-1 activity in vitro and in vivo.

### Tumor Necrosis Factors and Receptors Therefor

Tumor necrosis factors (TNFs) are pleiotropic cytokines that are primary modifiers of the inflammatory and immune reactions of animals produced in response to injury or infection. Two forms of TNF, designated TNF-α (or cachectin) and TNF-β (or lymphotoxin), have been described. These forms share 30% sequence similarity and compete for binding to the same receptors. TNFs play a necessary and beneficial role as mediators of host resistance to infections and tumor formation. Over production or inappropriate expression of these factors can lead to a variety of pathological conditions, including wasting, systemic toxicity, and septic shock (see, Beutler et al. (1988) Ann. Rev. Biochem.57:505; and Vilcek et al. (1991) J. Biol. Chem. 266:7313).

The actions of TNFs are produced subsequent to binding of the factors to cell surface receptors. Two distinct TNF receptors have been identified and cloned. Virtually all cell types studied show the presence of one or both of these receptor types. One receptor type, termed TNFR-II (Type A, Type α, 75 kDa or utr antigen), has an apparent molecular weight of 75 kDa. The gene for this receptor encodes a presumptive transmembrane protein of 439 amino acid residues (Dembic et al. (1990) Cytokine 2:231; Tartaglia etal. (1992) Immunol. Today 13:151). The other receptor type, termed TNFR-I (Type B, Type β, 55 kDa or htr antigen) has an apparent molecular weight of about 55 kDa. The gene for this protein encodes a transmembrane protein of 426 amino acid residues (Schall et al. (1990)Cell 61:361; Loetscher et al. (1990) Cell 61:351; Tartaglia etal. (1992) Immunol. Today 13:151). Both receptor types show high affinity binding of either TNF-α or TNF-62 . The two receptor types are immunologically distinct but their extracellular domains show similarities in the pattern of cysteine residue locations in four domains (Dembic et al. (1990) Cytokine 2:231).

Soluble TNF binding proteins in human serum and urine (Seckinger et al. (1989) J. Biol. Chem. 264:11966; Olsson et al. (1989) Eur. J. Haematol. 42:270; and Engelmann et al. (1990) J. Biol. Chem. 265:1541) that can neutralize the biological activities of TNF-α and TNF-β have been identified. Two types have been identified and designated sTNF RI (or TNF BPI) and sTNF RIh (or TNF BPII). These soluble forms are truncated forms of the two types of TNF receptors. The soluble receptor forms apparently arise as a result of shedding of the extracellular domains of the receptors, and concentrations of about 1-2 ng/mL are found in the serum and urine of healthy subjects (Aderka et al. (1992) Lymphokine and Cytokine Res. 11:157; Chouaib et al. (1991) Immunol. today 12:141). The levels of the soluble receptors vary from individual to individual but are stable over time for given individuals (Aderka et al. (1992) Lymphokine and Cytokine Res. 11:157).

The physiological role of the soluble TNF receptors is not known. It is known that both types of soluble receptors can bind to TNF in vitro and inhibit its biological activity by competing with cell surface receptors for TNF binding.

### Hemorrhagic Virus Desease and Disorders

A syndrome referred to as viral hemorrhagic fever is caused by one of several RNA viruses that include members of the viral families of Arenaviridae, Bunyaviridae, Filoviridae and Flaviviridae (see, e.g., Peters et al., Textbook of human virology (Belshe, ed.), Mosby Year Book, pp. 699-712 (1991)). Pronounced hemorrhage manifestations are characteristic of these fevers as well as disseminated intravascular coagulation (DIC), generalized shock, and a high mortality rate (30%-90%) (Fisher-Hoch et al., J. Infect. Dis., 1523:887-894 (1985); Fisher-Hoch, Rev. Med. Virol., 3:7-13 (1993); Murphy et al., Virology (Fields and Knipe, eds.), Raven, N.Y., pp. 936-942 (1990)). Despite some understanding of the progress of these diseases and responses, there are few, if any, effective treatments.

Due to the severity and breadth of viral hemorrhagic diseases and other disorders associated with a deleterious immune response, there is a great need for effective treatments of such diseases, disorders and conditions. Therefore, it is an object herein to provide treatments for such diseases and disorders.

### SUMMARY OF THE INVENTION

Methods and compositions for treating disorders and diseases involving acute inflammatory responses are provided. The methods and composition provided herein are used to treat various types viral and infectious diseases and other diseases, conditions and disorders, including but are not limited to, viral hemorrhagic diseases and other acute infectious diseases, sepsis, cachexia, rheumatoid arthritis and other autoimmune disorders, acute cardiovascular events, chronic myelogenous leukemia and transplanted bone marrow-induced graft-versus-host disease, septic shock, immune complex-induced colitis, cerebrospinal fluid inflammation, autoimmune disorders, multiple sclerosis and other such disorders. Other disorders, conditions and diseases include, but are not limited to, trauma, such as polytrauma, burns, major surgery; systemic inflammatory response syndrome (SIRS); adult respiratory distress syndrome (ARDS); acute liver failure; inflammatory bowel disease, Crohn's disease and other such disorders.

In a particular embodiment, methods and compositions for treating viral and other infectious diseases, particularly bacterial sepsis and viral hemorrhagic diseases or disorders, particularly those viral hemorrhagic diseases or disorders caused by infection with a Bunyaviridaea, a Filoviridae, a Flaviviridae, or an Arenaviridae virus, and other disorders, such as sepsis, particularly that associated with exposure to gram negative bacterial endotoxins, and shock, including that associated with trauma, and infections, such as parasitic infections, that are characterized by an immunologic response, particularly an acute inflammatory responses, involving cellular activation, including production of tumor necrosis factors, interleukins, chemokines and interferons are provided.

Compositions for effecting such treatment are also provided. Tetracycline and tetracycline-like compounds and the blood-derived compositions for effecting such treatment are provided herein. It is shown herein that tetracycline compounds and tetracycline-like compounds as defined herein can be used for treatment of disorders involving acute inflammatory responses. The tetracycline and tetracycline-like compounds are used to treat the disorders and also to produce blood product compositions from donors for the treatment of the disorders. The blood product compositions and the tetracycline and tetracycline-like compounds can be used together or each can be used for treatment of these disorders.

Also provided are methods of preparing blood or fractions thereof for use in preparing compositions for treatment of acute inflammatory conditions, disorders and diseases, by treating the blood or fraction thereof in vitro or in vivo with a compound that is tetracycline or tetracycline-like compound. Hence methods for preparation of blood-derived compositions for treatment of diseases, conditions and disorders characterized by or involving an inflammatory immune response are provided. Methods for such production are provided. The compositions are produced either in vitro or in vivo or a combination thereof by contacting blood or blood fraction or product with a tetracycline and/or tetracycline-like compound for a sufficient time to result in at least about a 3-fold increase in the level of a selected cytokine receptors, such as IL-1 receptors and/or TNF receptors. Hence, the level of receptors, such as IL-1 receptors and/or soluble TNF receptors, in the blood or blood fraction or product is tested before and after contacting with the tetracycline or tetracycline-like compound.

In particular, a method for producing a cytokine-receptor-enriched blood product by treating blood or a fraction thereof with a tetracycline or tetracycline-like compound; and harvesting, by methods described herein or known to those of skill in the art, fractions thereof, and selecting the cytokine-receptor enriched plasma, serum or other fraction. The resulting compositions are enriched for cytokine receptors compared to the blood prior to treatment. The receptors of interest include soluble tumor necrosis factor (TNF) receptors and/or interleukin-1RA (IL-1RA) receptors. Contacting the blood or fraction thereof can be effected in vitro or in vivo. Hence a method for producing cytokine-receptor-enriched compositions by treating white blood cells in vitro with a tetracycline or tetracycline-like compound to induce receptor expression; and collecting extracellular medium is provided.

The resulting compositions and use thereof for treatment of conditions, diseases and disorders associated with acute inflammatory responses are provided.

Processes for producing compositions suitable for treating viral hemorrhagic diseases or disorders are provided. These processes include some or all of the steps of: a) administering one or more tetracycline compounds to a mammal; b) collecting blood from the mammal; and c) recovering serum or plasma from the collected blood to thereby produce a composition for use in treating the disorders or diseases. Such compositions, which are preferably derived from the plasma, can be used to treat viral hemorrhagic diseases or disorders, particularly those viral hemorrhagic diseases or disorders caused by infection of a Bunyaviridae, a Filoviridae, a Flaviviridae, or an Arenaviridae virus. These compositions also can be used to treat any disorder involving a cytotoxic response, including but not limited to sepsis and endotoxic shock. The plasma (or serum portion) may be further fractionated and fractions that possess the desired therapeutic activity (treatment of symptoms associated with the viral infection, shock or other such disorder) identified empirically and formulated, if necessary, into compositions for treatment of the mammal. For humans, the plasma (or blood) is preferably derived from a human treated with a tetracycline compound.

In particular, plasma or derivatives of the plasma produced by administering a tetracycline or tetracycline-like compound, and then isolating the fraction rich in released soluble factors, such as II-1 receptors and TNF-1 receptors. The plasma fraction is for treating acute events, including the viral infections, and cardiovascular events. Hence compositions containing these soluble receptors, immunoattenuating factors, are provided. These are produced by administering a tetracycline compound or a tetracycline-like compound to induce the factors, harvesting the plasma, optionally enriching the plasma for these factors that sop up inflammatory factors. The resulting composition is administered.

Also provided are the resulting blood-derived compositions, and methods of treatment of treating viral hemorrhagic diseases or disorders and other diseases involving a cytotoxic response in which TNF or IL-1 or both or other cytokines or receptors therefor are elevated, by administering the blood-derived compositions.

Also provided are methods of treatment of these conditions, diseases and disorders (collectively referred to as conditions). The compositions are administered to a mammal with a condition associated with or characterized by an acute inflammatory response. These compositions can be administered in combination with tetracycline and/or tetracycline-like compounds and also optionally in combination with other therapies for each disorder. The combination therapies may be administered simultaneously, consecutively, intermittently or in any desired or effective order. The may be repeated as needed.

Hence in certain embodiments, tetracycline and tetracycline-like compounds other related compounds and the blood-derived compositions provided herein are used to treat various types viral and infectious diseases, particularly viral hemorrhagic diseases or disorders, particularly those viral hemorrhagic diseases or disorders caused by infection with a Bunyaviridae, a Filoviridae, a Flaviviridae, or an Arenaviridae virus, and other disorders, such as sepsis, particularly that associated with exposure to gram negative bacterial endotoxins, and shock, including that associated with trauma, and infections, such as parasitic infections, that are characterized by an immunologic response, particularly acute inflammatory responses, involving cellular activation, including production tumor necrosis factors, interleukins, chemokines and interferons. Hence the tetracycline and tetracycline-like compounds and the blood-derived compositions provided herein are used to treat conditions and disorders, including but are not limited to, sepsis, cachexia, rheumatoid arthritis, chronic myelogenous leukemia and transplanted bone marrow-induced graft-versus-host disease, septic shock, immune complex-induced colitis and cerebrospinal fluid inflammation.

Encompassed within the methods are the uses of any tetracycline compound, or derivatives thereof, or a mixture thereof, and tetracycline-like compounds that can alleviate, reduce, ameliorate, or prevent viral hemorrhagic diseases or disorders and other acute inflammatory response; or place or maintain in a state of remission of clinical symptoms or diagnostic markers associated with such diseases or disorders.

Of particular interest are methods of treatment for viral hemorrhagic diseases and disorders caused by infection with a Bunyaviridae, a Filoviridae, a Flaviviridae, or an Arenaviridae virus. The compounds and compositions provided herein can be used alone or in combination with other treatments for hemorrhagic disorders. Viruses that cause hemorrhagic diseases include, but are not limited to, Bunyaviridae, a Filoviridae, a Flaviviridae, and Arenaviridae viruses. The Bunyaviridae viruses include, but are not limited to, bunyavirus (Bunyamwera, Bwamba, California, Capim, Guama, phlebovirus koongol, patois, simbu and tete viruses), sandfly fever virus, Rift Valley fever virus of sheep and ruminants, Nairovirus, Crimean-Congo hemorrhagic fever virus, Uukuvirus, Uukuniemi virus, Hantaan virus and Korean hemorrhagic fever virus. In particular, the Bunyaviridae viruses include, Crimean-Congo hemorrhagic fever virus, Hantaan virus and Korean hemorrhagic fever virus. The Filoviridae viruses include, but are not limited to, ebola virus, such as the Zaire, Sudan, Reston and Ivory Coast subtypes, and Marburg viruses. Other Flaviviridae virus include flavivirus, Brazilian encephalitis virus, Bussuquara virus, Dengue virus, iiheus virus, Israel turkey meningoencephalitis virus, Japanese B encephalitis virus, Kunjin virus, Kyasanur forest disease virus, Langat virus, Louping ill virus, Modoc virus, Murray valley encephalitis virus, Ntaya virus, omsk hemorrhagic fever virus, powassan virus, St. Louis encephalitis virus, spondwnei virus, tick-borne encephalitis, Uganda S virus, US bat salivary gland virus, wesselsbron virus, West Nile fever virus, yellow fever virus, Zika virus, European tick-borne encephalitis, Far Eastern tick-borne encephalitis virus, Russian tick-borne encephalitis, and Dengue virus, including but are not limited to, Dengue type 1, Dengue type 2, Dengue type 3 and Dengue type 4 virus. The Arenaviridae viruses include, but are not limited to, Junin virus, Lassa virus such as the Josiah strain or Nigerian strain, Machupo virus, Pichinde virus, lymphocytic choriomeningitis virus, Lassa fever virus and arenavirus.

Provided herein are combinations, preferably in the form of pharmaceutical compositions, including one or more tetracycline compound(s) and one or more anti-hemorrhagic virus treatments. The combinations are typically pharmaceutical compositions that include a tetracycline compound formulated for single dosage administration, and an agent, other than a tetracycline compound, that is an anti-hemorrhagic viral agent, such as a vaccine, antibody or other pharmaceutical. The compound and agent can be administered separately, such as sequentially, or can be administered intermittently, or together as two separate compositions or as a mixture in a single composition. The dosage of each can be empirically determined, but is generally the dosage of a agent normally used to treat the hemorrhagic viral infection, and an amount of a tetracycline compound sufficient to further enhance treatment, or sufficient when used alone to reduce or ameliorate or in some manner reduce symptoms. The combinations can be packaged as kits.

In a preferred embodiment, the combination contains a single composition containing the tetracycline compound and anti-hemorrhagic virus agent formulated for oral delivery or two compositions, one containing a tetracycline compound and the other an anti-viral-hemorrhagic agent, where each is in a pharmaceutically acceptable carrier or excipient in tablet, capsule, or other single unit dosage form. Alternatively, the two components can be mixed in a single composition. In other embodiments, the compositions are formulated for rectal, topical, inhalation, buccal (e.g., sublingual), parenteral (e.g., subcutaneous, intramuscular, intradermal, or intravenous including bolus injection) and transdermal administration. Specific therapeutic regimens, pharmaceutical compositions, and kits are also provided.

Also provided is a method for treating viral hemorrhagic diseases or disorders in mammals, including humans, particularly those viral hemorrhagic diseases or disorders caused by infection of any virus causing such disease or disorder, including but not limited to a Bunyaviridae, a Filoviridae, a Flaviviridae, or an Arenaviridae virus, by administrating a therapeutically effective and non-lethal amount of one or more tetracycline compound(s).

Tetracycline compounds include, but are not limited to chlortetracycline, demeclocycline, doxycycline, methacycline, minocycline, oxytetracycline and tetracycline. Tetracycline-like compounds are those that share the property of altering folic acid metabolism in bacteria. Such compounds include thalidomide and sulfa drugs.

Anti-hemorrhagic virus treatments include treatment protocols and agents that are used to treat hemorrhagic viral diseases or ameliorate the symptoms thereof. Such agents include, but are not limited to agents that inhibit interleukin-1 (IL-1) and agents that inhibit TNF. Other anti-hemorrhagic viral agents, include, but are not limited to, anti-viral vaccines, anti-viral antibodies, a viral-activated immune cells, such as activated cytotoxic cells, and viral-activated immune serum.

Agents that inhibit IL-1, include, but are not limited to, anti-IL-1 antibodies, anti-IL-1 receptor antibodies, IL-1 receptor antagonists, an IL-1 production inhibitors, IL-1 receptor production inhibitors, and IL-1 releasing inhibitors.

IL-1 receptor antagonists include, but are not limited to, the IL-1 receptor antagonist (IL-1Ra), IL-1 receptor intracellular ligand protein, a Type II IL-1 receptor, a soluble IL-1 receptor, a non-biologically active (i.e., non-functional) mutein of IL-1 that binds to IL-receptors, a non-functional mutein of IL-1 receptor and small molecule antagonists, such as histamine antagonist, a aryl-or heteroaryl-1-alkyl-pyrrole-2-carboxylic acid compound and a 5-lipoxygenase pathway inhibitor.

IL-1 production inhibitors include antisense oligonucleotides, 5-hydroxy- and 5-methoxy-2-amino-pyrimidines, a 3-substituted-2-oxindole-1-carboxamide, a 4,5-diaryl-2(substituted)imidazole and a 2-2′-[1,3-propan-2-onediyl-bis(thio)]bis-1-H-imidazole. IL-1 releasing inhibitors include IL-1 converting enzyme inhibitors, such as, but are not limited to, a peptide based interleukin-1 beta converting enzyme inhibitor, a pyridazinodiazepine, SDZ 224-015, an aspartate-based inhibitor, an aspartyl alpha-((1-phenyl-3-(trifluoromethyl)-pyrazol-5-yl)oxy)methyl ketone, L-741,494, TX, CPP-32 and CMH-1.

Agents that inhibit TNF include, but are not limited to, anti-TNF antibody (polyclonal or monoclonal), an anti-TNF receptor antibody (polyclonal or monoclonal), a TNF receptor antagonist, a TNF production inhibitor, a TNF receptor production inhibitor and a TNF releasing inhibitor. Anti-TNF monoclonal antibodies, include, but are not limited to, Mabp55r, Mabp75r, 3B10, h3B10-9, MAK 195F, CA2 and CDP571. Other TNF receptor antagonists include, but are not limited to, soluble TNF receptor, a non-functional mutein that binds to the TNF receptor, but does not exhibit TNF biological activity, a non-functional mutein of TNF and small molecule antagonists, such as but are not limited to, a mercapto alkyl peptidyl compound, an arylsulfonyl hydroxamic acid derivative, a salt of an alkaline-earth metal, a pentoxifylline, a hydroxamic acid compound, a retinoic acid, a histamine antagonist, a leflunomide, a 1-Alkoxy-2-(alkoxy- or cycloalkoxy-)-4-(cyclothioalkyl- or cyclothio-alkenyl-)benzene, a vinigrol, a cyclohexene-ylidene derivative, a quinazoline compound and BN 50739. Other TNF receptor antagonists include, but are not limited to, TNF receptor death domain ligand protein, a tumor necrosis factor binding protein (TNF-BP), a TNF receptor-IgG heavy chain chimeric protein, a bacterial lipopolysaccharide binding peptide derived from CAP37 protein and a Myxoma virus T2 protein. TNF production inhibitors, include antisense oligonucleotides, quinoline-3-carboxamide compounds and derivatives of 2-pyrrolidinone. TNF releasing inhibitors include isoxazoline compounds and catechol diether compounds.

Methods herein are for stimulating release of the receptors such as, but not limited to, TNF-α, IL-1 receptors and other soluble factors that down-regulate excessive T-helper 1 (TH1) response, that is stimulated by tetracycline administration. The receptors are those that bind to and/or inhibit inflammatory factors that are released in various inflammatory conditions, viral infections, bacterial infections, and conditions associate with fungal and parastitic infections, inflammatory responses, such as asthma, sepsis, rheumatoids, atherosclerosis, inflammatory responses associated with injury, and cardiovascular events and events related to cell activation, i.e., acute events brought on by excessive release of inflammatory factors.

### DETAILED DESCRIPTION OF THE INVENTION

Particular compositions, combinations, kits and methods are described in the sections and subsections as follows:

A. Definitions

B. Combinations and Kits and Compositions for Treatment of Acute Inflammatory Responses

1. Tetracycline-like compounds

2. Tetracycline compounds

a. Anti-inflammatory activity of tetracyclines

b. Exemplary tetracycline compounds

(1) Chlortetracycline

(2) Demeclocycline

(3) Doxycycline

(4) Methacycline

(5) Minocycline

(6) Oxytetracycline

(7) Tetracycline

(8) Other Chemically-Modified Tetracyclines

C. Hemorrhagic Viruses and the Immune Response

D. Parmaceutical Compositions, Formulation and Modes of Administration thereof

1. Anti-viral-hemorrhagic agents

a. lnterleukin-1 (II-1) inhibitors

b. Tumor necrosis factor (TNF) inhibitors

c. Anti-viral vaccine, antibody and virally-activated immune cells and serum

(1) Anti-viral vaccine

(a) Anti-Bunyaviridae Vaccine

(b) Anti-Filoviridae Vaccine

(c) Anti-Flaviviridae Vaccine

(d) Anti-Arenaviridae Vaccine

(2) Anti-viral antibodies

(a) Anti-Bunyaviridae Antibody

(b) Anti-Filoviridae Antibody

(c) Anti-Flaviviridae Antibody

(d) Anti-Arenaviridae Antibody

(3) Viral-activated immune cell and serum

(4) Small molecule anti-viral agents

2. Formulation and routes of administration

E. Blood-derived Compostions and Methods of Treatment

1. Blood-derived compositions and processes for producing compositions for treating diseases and disorders characterized by or associated with acute inflammatory responses

a. Preparation of Serum and Plasma

b. Further Fractionation of Plasma

(1) Preparation of Albumin-Containing Fraction

(2) Preparaton of Globulin-Containing Fraction

(3) Preparation of AHF-Containing Fraction

(4) Preparation of Fraction Containing Soluble IL-1 Receptor or Soluble TNF Receptor

C. Methods of treatment using the resulting blood-derived compositions

F. Viral hemorrhagic Disease or Disorder and Diagnosis Thereof

1. Bunyaviridae Virus Infection

2. Filoviridae Virus Infection

3. Flaviviridae Virus Infection

4. Arenaviridae Virus Infection

G. Examples

A. Definitions

Unless defined otherwise, all technical and scientific terms used herein have the same meaning as is commonly understood by one of skill in the art to which this invention belongs. Where permitted, all patents, applications, published applications and other publications and sequences from GenBank and other data bases referred to throughout the disclosure herein are incorporated by reference in their entirety.

As used herein, “tetracycline compound” refers to any compound having the activity of a tetracycline, prodrugs, salts, esters or other derivatives of tetracycline, preferably in a pharmaceutically acceptable form, known to those of skill in the art.

Tetracycline, which is well known to those of skill in the art, has the structure:

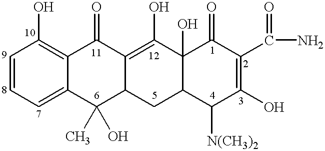

It is intended herein for the the term “tetracycline” to encompass all pharmaceutically active species of tetracycline compounds, solutions thereof and mixtures thereof, prodrugs thereof and any drug recognized as a tetracycline. Tetracycline includes forms, such as hydrated forms, and compositions such as aqueous solutions, hydrolyzed products or ionized products of these compounds; and these compounds may contain different numbers of attached water molecules. Thus, as used herein, the term tetracycline compound encompasses all derivatives and analogs and modified forms thereof, including but not limited to, those set forth herein. Tetracycline and tetracycline-like compounds include, but are not limited to aspirin, aureomycin, apicycline, chlortetracycline, clomocycline, demeclocyline, guamecycline, lymecycline, meclocycline, methacycline, minocycline, oxytetracycline, penimepicycline, pipacycline, rolitetracycline, sancycline, and senociclin, as well as any others falling within the above formula. Also included among tetracycline-like compounds are compounds that alter bacterial folic acid metabolism, such as sulfa drugs, including sulfonamides, and thalidomide. Such compounds can be identified by their ability to alter bacterial folic acid metabolism.

As used herein, tetracycline-like compounds, such as aureomycin, sulfa drugs and thalidomide, refer to compounds that have the activity of tetracycline in the methods herein. Such compounds can be identified by their ability to alter folic acid metabolism in bacterial species, particularly those in which tetracycline alters folic acid metabolism.

As shown herein, a tetracycline and tetracycline-like compound herein is a compound that stimulates release of soluble factors in the blood that attenuate inflammatory responses.

Any tetracycline compound(s), when used alone or in combination with other compounds, that can alleviate, reduce, ameliorate, prevent, or place or maintain in a state of remission of clinical symptoms or diagnostic markers associated with viral hemorrhagic diseases or disorders, particularly those viral hemorrhagic diseases or disorders caused by infection of a Bunyaviridae, a Filoviridae, a Flaviviridae, or an Arenaviridae virus, are intended for use in the methods, compositions and combinations provided herein.

As used herein, an anti-hemorrhagic virus treatment refers to any treatment designed to treat hemorrhagic viral infections by lessening or ameliorating the symptoms. Treatments that prevent the infection or lessen its severity are also contemplated. An anti-hemorrhagic virus agent (used interchangeable with “anti-viral-hemorrhagic agent”) refers to any agents used in the treatment. These include any agents, when used alone or in combination with other compounds, that can alleviate, reduce, ameliorate, prevent, or place or maintain in a state of remission of clinical symptoms or diagnostic markers associated with viral hemorrhagic diseases or disorders, particularly those viral hemorrhagic diseases or disorders caused by infection of a Bunyaviridae, a Filoviridae, a Flaviviridae, or an Arenaviridae virus, can be used in methods, combinations and compositions provided herein. Non-limiting examples of anti-viral-hemorrhagic agents include interleukin-1 (IL-1) inhibitors, tumor necrosis factor (TNF) inhibitors, anti-viral vaccines, anti-viral antibodies, viral-activated immune cells and viral-activated immune sera.

As used herein, anti-hemorrhagic virus agent (anti-viral-hemorrhagic agent) or anti-hemorrhagic virus treatment does not encompass “tetracycline compound” or use thereof for treatment, but encompasses all agents and treatment modalities known to those of skill in the art to ameliorate the symptoms of a hemorrhagic viral infection.

As used herein, a cytokine is a factor, such as lymphokine or monokine, that is produced by cells that affect the same or other cells. A “cytokine” is one of the group of molecules involved in signaling between cells during immune responses. Cytokines are proteins or peptides; and some are glycoproteins.

As used herein, “interleukin (IL)” refers to a large group of cytokines produced mainly by T cells, although some are also produced by mononuclear phagocytes, or by tissue cells. They have a variety of functions, but most of them are involved in directing other cells to divide and differentiate. Each interleukin acts on specific, limited groups of cells which express the correct receptors for that cytokine.

As used herein, “interleukin-1 (IL-1)” refers to interleukins made by certain antigen presenting cells (APCs) that, along with IL-6, act as co-stimulatory signals for T cell activation. The IL-1 gene family includes IL-1α, IL-1β and IL-1 receptor antagonist (IL-1Ra) (Dinarello, Eur. Cytokine Netw., 5(6):517-522 (1994)). Each member is first synthesized as a precursor protein; the precursors for IL-1 (proIL-1α and proIL-1β) have molecular weights of about 31,000 Da. The prolL-1a and mature 17,000 Da IL-1α are biologically active whereas the proIL-1β requires cleavage to a 17,000 Da peptide for optimal biological activity. The IL-IRa precursor has a leader sequence and is cleaved to its mature form and secreted like most proteins. IL-1α and IL-1β are potent agonists where IL-1Ra is a specific receptor antagonist. Moreover, IL-IRa appears to be a pure receptor antagonist with no agonist activity in vitro or in vivo. Although IL-1Ra is a secreted protein, there is another form of this molecule which is retained inside cells. It is called “intracellular” (ic) IL-1Ra. IcIL-1Ra results from alternate mRNA splice insertion of the IL-1Ra gene replacing the exon coding for the signal peptide. The forms of IL-1Ra are functionally indistinguishable.

Thus, reference, for example, to “IL-1” encompasses all proteins encoded by the IL-1 gene family including IL-1α, IL-1β, IL-1Ra and icIL-lRa, or an equivalent molecule obtained from any other source or that has been prepared synthetically. It is intended to encompass IL-1 with conservative amino acid substitutions that do not substantially alter its activity. Suitable conservative substitutions of amino acids are known to those of skill in this art and may be made generally without altering the biological activity of the resulting molecule. Those of skill in this art recognize that, in general, single amino acid substitutions in non-essential regions of a polypeptide do not substantially alter biological activity (see, e.g., Watson et al. Molecular Biology of the Gene, 4th Edition, 1987, The Bejacmin/Cummings Pub. co., p.224).

Such substitutions are preferably made in accordance with those set forth in TABLE 1 as follows:

TABLE 1

| Original residue | Conservative substitution |
|---|---|
| Ala (A) | Gly; Ser |
| Arg (R) | Lys |
| Asn (N) | Gln; His |
| Cys (C) | Ser |
| Gln (Q) | Asn |
| Glu (E) | Asp |
| Gly (G) | Ala; Pro |
| His (H) | Asn; Gln |
| Ile (I) | Leu; Val |
| Leu (L) | Ile; Val |
| Lys (K) | Arg; Gln; Glu |
| Met (M) | Leu; Tyr; Ile |
| Phe (F) | Met; Leu; Tyr |
| Ser (S) | Thr |
| Thr (T) | Ser |
| Trp (W) | Tyr |
| Tyr (Y) | Trp; Phe |
| Val (V) | Ile; Leu |

Other substitutions are also permissible and may be determined empirically or in accord with known conservative substitutions.

As used herein, the amino acids, which occur in the various amino acid sequences appearing herein, are identified according to their well-known, three-letter or one-letter abbreviations. The nucleotides, which occur in the various DNA fragments, are designated with the standard single-letter designations used routinely in the art.

As used herein, “IL-1 inhibitor” encompasses any substances that prevent or decrease production, post-translational modification(s), maturation, or release of IL-1, or any substances that interfere with or decrease the efficacy of the interaction between IL-1 (see, e.g., SEQ ID Nos. 1 and 2) and IL-1 receptor (see, e.g., SEQ ID Nos. 3 and 4). Preferably, the IL-1 inhibitor is an anti-IL-1 antibody, an anti-IL-1 receptor antibody, an IL-1 receptor antagonist, an IL-1 production inhibitor, an IL-1 receptor production inhibitor and an IL-1 releasing inhibitor.

As used herein, the terms “a therapeutic agent”, “therapeutic regimen”, “radioprotectant”, “chemotherapeutic” mean conventional drugs and drug therapies, including vaccines, which are known to those skilled in the art. “Radiotherapeutic” agents are well known in the art.

As used herein, “interleukin-1 converting enzyme (ICE)” refers to a protease that processes the IL-1β precursor (pIL-1β) to the mature IL-1β (mIL-1β) (U.S. Pat. No. 5,552,536). ICE generates fully active mIL-1i by cleaving pIL-1β between Asp₁₁₆ and Ala₁₁₇, a unique site for pheromone processing. The sequence around this cleavage site, -Tyr-Val-His-Asp-Ala-, is evolutionarily conserved in all known pIL-1β polypeptides. Active human ICE is a heterodimer with a 1:1 stoichiometric complex of p20 and p10 subunits. Cloned cDNA have revealed that ICE is constitutively expressed as a 45 kDa proenzyme (p45) composed of a 14 kDa prodomain, followed by p20 which contains the active site CyS₂₈₅, a 19 residue connecting peptide that is not present in the mature enzyme, and p10, a required component of the active enzyme. The mature subunits are flanked by Asp-X sequences. Mutational analysis of these sites and expression in heterologous systems indicates that the generation of active enzyme is autocatalytic. Murine and rat ICE have also been cloned and show a high degree of sequence similarity including these structural motifs.

As used herein, “tumor necrosis factor (TNF)” refers to a group of proinflammatory cytokines encoded within the major histocompatibility complex (MHC). TNF family members include TNFα (also known as cachectin) and TNFβ (also known as lymphotoxin). Complementary cDNA clones encoding TNFα (Pennica et al., Nature, 312:724 (1984)) and TNFβ (Gray et al., Nature, 312:721 (1984)) have been isolated. Therefore, reference, for example, to “TNF” encompasses all proteins encoded by the TNF gene family including TNFα and TNFβ, or an equivalent molecule obtained from any other source or that has been prepared synthetically. It is intended to encompass TNF with conservative amino acid substitutions that do not substantially alter its activity.

As used herein, “TNF inhibitor” encompasses any substances that prevent or decrease production, post-translational modification(s), maturation, or release of TNF, or any substances that interfere with or decrease the efficacy of the interaction between TNF (see, e.g., SEQ ID Nos. 14 and 15) and TNF receptor (see, SEQ ID Nos. 16 and 17). Preferably, the TNF inhibitor is an anti-TNF antibody, an anti-TNF receptor antibody, a TNF receptor antagonist, a TNF production inhibitor, a TNF receptor production inhibitor and a TNF releasing inhibitor.

Native TNF receptors are characterized by distinct extracellular, transmembrane and intracellular domains. Two distinct TNF receptors of about 55 kd (“TNF-R1”) and about 75 kDa (“TNF-R2”) have been identified. Numerous studies have demonstrated that TNF-R1 is the receptor which signals the majority of the pleiotropic activities of TNF. The domain required for signaling cytotoxicity and other TNF-mediated responses has been mapped to the about 80 amino acids near the C-terminus of TNF-R1. This domain is therefore termed the “death domain” (“TNF-R death domain” and “TNF-R1-DD”) (see, U.S. Pat. No. 5,852,173; and Tartaglia et al., Cell, 74:845-853 (1993)).

As used herein, “antisense polynucleotides” refer to synthetic sequences of nucleotide bases complementary to mRNA or the sense strand of double stranded DNA. A mixture of sense and antisense polynucleotides under appropriate conditions leads to the binding of the two molecules, or hybridization. When these polynucleotides bind to (hybridize with) mRNA, inhibition of protein synthesis (translation) occurs. When these polynucleotides bind to double stranded DNA, inhibition of RNA synthesis (transcription) occurs. The resulting inhibition of translation and/or transcription leads to an inhibition of the synthesis of the protein encoded by the sense strand.

As used herein, an antisense oligonucleotide that contains a sufficient number of nucleotides to inhibit translation of an mRNA, such as an interleukin-1 (IL-1), such as IL-1α, or TNF. An antisense oligonucleotide refers to any oligomer that prevents production or expression of, for example, IL-1 polypeptide. The size of such an oligomer can be any length that is effective for this purpose. In general, the antisense oligomer is prepared in accordance with the nucleotide sequence of a portion of the transcript of interest (i.e., IL-1 and TNF) that includes the translation initiation codon and contains a sufficient number of complementary nucleotides to block translation.

As used herein, “vaccine” refers to any composition for active immunological prophylaxis. A vaccine may be used therapeutically to treat a disease, or to prevent development of a disease or to decrease the severity of a disease either proactively or after infection. Non-limiting examples of vaccines include, but are not limited to, preparations of killed microbes of virulent strains or living microbes of attenuated (variant or mutant) strains, or microbial, fungal, plant, protozoa, or metazoa derivatives or products. “Vaccine” also encompasses protein/peptide and nucleotide based vaccines.

As used herein, “cytotoxic cells” refers to cells that kill virally infected targets expressing antigenic peptides presented by MHC class I molecules.

As used herein, “treating hemorrhagic viral diseases or disorders” means that the diseases and the symptoms associated with the hemorrhagic viral diseases or disorders are alleviated, reduced, ameliorated, prevented, placed in a state of remission, or maintained in a state of remission. Additionally, as used herein, “a method for treating hemorrhagic viral diseases or disorders” means that the hallmarks of hemorrhagic viral diseases or disorders are eliminated, reduced or prevented by the treatment. Non-limiting examples of the hallmarks of the viral hemorrhagic diseases or disorders include disseminated intravascular coagulation (DIC), generalized shock, and the highest mortality rate (30%-90%).

As used herein, a blood-derived composition (or immune composition) refers to the composition produced from the blood of mammals treated with a tetracycline and/or tetracycline-like compound. It also refers to the compositions produced by in vitro treatment of blood or a blood fraction with a tetracycline or tetracycline-like compound. These blood-derived compositions are for treating, not only the hemorrhagic disorders, but also for alleviating any disorder involving a deleterious immune response, such as septic shock and endotoxic shock.

The immune response to certain infectious agents, such as viruses, parasites and bacteria, and in certain diseases and conditions, activate cells and products thereof that have deleterious consequences. For example, LPS (lipopolysaccharide) binds to immunoglobin M and this complex activates the complement system with the release of C3b, which material in turn activates the polymorphonuclear leukocytes (PMN), monocytes, neutrophils, macrophage and endothelial cells. The activation of these substances stimulates the release of several mediators of septic shock including tumor necrosis factor (TNF-a) interleukin-1 (IL-1) and other interleukins including IL-6 and IL-8, platelet-activating factor (PAF), prostaglandins and leukotrienes (see, e.g., (1991) Ann. Intern. Med. 115: 464-466 for a more comprehensive listing). Of these, the two cytokines TNF-α and IL-1 lead to many of the physiologic changes which eventuate into septic shock.

As used herein, an acute inflammatory disease, condition or disorder, refers to any condition, disease or disorder in which a deleterious elevation of cytokines and other inflammatory mediators occurs. For purposes herein, disease, condition and disorder refer to the manifestation of such elevation. In general a disease is caused by an infectious agent, a disorder refers to a disease that does not have a known infectious agent as a cause and a condition is used to capture all such symptoms and characteristics associated with acute inflammatory responses. They are referred to herein in the alternative to ensure that all are encompassed.

As used herein, “serum” refers to the fluid portion of the blood obtained after removal of the fibrin clot and blood cells, distinguished from the plasma in circulating blood.

As used herein, “plasma” refers to the fluid, noncellular portion of the blood, distinguished from the serum obtained after coagulation.

As used herein, “albumin” refers to a type of protein, varieties of which are widely distributed throughout the tissues and fluids of plants and animals, especially animal blood. Albumin are soluble in pure water, precipitable from solution by strong acids and coagulable by heat in acid or neutral solution.

As used herein, “globulin” refers to a family of proteins precipitated from plasma (or serum) by half-saturation with ammonium sulfate. Globulin may be further fractionated by solubility, electrophoresis, ultracentrifugation, and other separation methods into many subgroups, the main groups being α-, β-, and γ-globulins.

As used herein, “antihemophilic factor (AHF)” refers the fraction of blood that contains Factor VIII and/or von Willebrand's factor, which are important in the blood clotting mechanism (see, e.g., U.S. Pat. No. 4,435,318). Factor VIII serves as a co-factor along with calcium and phospholipid to enable Factor IXₐ to cleave zymogen Factor X to thus activate Factor X, all being a part of the complex coagulation cascade system. Von Willebrand's factor (vWF) apparently acts in the aggregation of platelets which provide the necessary phospholipid. The absence of either of these factors may result in prolonged bleeding times. Factor V also serves an important role in the coagulation system by aiding activated Factor X in the cleavage of prothrombin to thrombin. (The Plasma Proteins, Vol. III, 2nd Ed., Structure, Function, Genetic Control (1977) (Academic Press, Inc., N.Y.) p. 422-544.)

As used herein, an effective amount of a compound for treating a particular disease is an amount that is sufficient to ameliorate, or in some manner reduce the symptoms associated with the disease. Such amount may be administered as a single dosage or may be administered according to a regimen, whereby it is effective. The amount may cure the disease but, typically, is administered in order to ameliorate the symptoms of the disease. Repeated administration may be required to achieve the desired amelioration of symptoms.

As used herein, pharmaceutically acceptable salts, esters or other derivatives of the conjugates include any salts, esters or derivatives that may be readily prepared by those of skill in this art using known methods for such derivatization and that produce compounds that may be administered to animals or humans without substantial toxic effects and that either are pharmaceutically active or are prodrugs.

As used herein, treatment means any manner in which the symptoms of a condition, disorder or disease are ameliorated or otherwise beneficially altered. Treatment also encompasses any pharmaceutical use of the compositions herein.

As used herein, amelioration of the symptoms of a particular disorder by administration of a particular pharmaceutical composition refers to any lessening, whether permanent or temporary, lasting or transient that can be attributed to or associated with administration of the composition.

As used herein, substantially pure means sufficiently homogeneous to appear free of readily detectable impurities as determined by standard methods of analysis, such as thin layer chromatography (TLC), gel electrophoresis and high performance liquid chromatography (HPLC), used by those of skill in the art to assess such purity, or sufficiently pure such that further purification would not detectably alter the physical and chemical properties, such as enzymatic and biological activities, of the substance. Methods for purification of the compounds to produce substantially chemically pure compounds are known to those of skill in the art. A substantially chemically pure compound may, however, be a mixture of stereoisomers or isomers. In such instances, further purification might increase the specific activity of the compound.

As used herein, a prodrug is a compound that, upon in vivo administration, is metabolized or otherwise converted to the biologically, pharmaceutically or therapeutically active form of the compound. To produce a prodrug, the pharmaceutically active compound is modified such that the active compound will be regenerated by metabolic processes. The prodrug may be designed to alter the metabolic stability or the transport characteristics of a drug, to mask side effects or toxicity, to improve the flavor of a drug or to alter other characteristics or properties of a drug. By virtue of knowledge of pharmacodynamic processes and drug metabolism in vivo, those of skill in this art, once a pharmaceutically active compound is known, can design prodrugs of the compound (see, e.g., Nogrady (1985) Medicinal Chemistry A Biochemical Approach, Oxford University Press, New York, pages 388-392).

As used herein, biological activity refers to the in vivo activities of a compound or physiological responses that result upon in vivo administration of a compound, composition or other mixture. Biological activity, thus, encompasses therapeutic effects and pharmaceutical activity of such compounds, compositions and mixtures. Biological activities may be observed in in vitro systems designed to test or use such activities. Thus, for purposes herein, the biological activity of a luciferase is its oxygenase activity whereby, upon oxidation of a substrate, light is produced.

As used herein, a receptor refers to a molecule that has an affinity for a given ligand. Receptors may be naturally-occurring or synthetic molecules. Receptors may also be referred to in the art as anti-ligands. As used herein, the receptor and anti-ligand are interchangeable. Receptors can be used in their unaltered state or as aggregates with other species. Receptors may be attached, covalently or noncovalently, or in physical contact with, to a binding member, either directly or indirectly via a specific binding substance or linker. Examples of receptors, include, but are not limited to: antibodies, cell membrane receptors surface receptors and internalizing receptors, monoclonal antibodies and antisera reactive with specific antigenic determinants [such as on viruses, cells, or other materials], drugs, polynucleotides, nucleic acids, peptides, cofactors, lectins, sugars, polysaccharides, cells, cellular membranes, and organelles.

Examples of receptors and applications using such receptors, include but are not restricted to:

a) enzymes: specific transport proteins or enzymes essential to survival of microorganisms, which could serve as targets for antibiotic [ligand] selection;

b) antibodies: identification of a ligand-binding site on the antibody molecule that combines with the epitope of an antigen of interest may be investigated; determination of a sequence that mimics an antigenic epitope may lead to the development of vaccines of which the immunogen is based on one or more of such sequences or lead to the development of related diagnostic agents or compounds useful in therapeutic treatments such as for auto-immune diseases

c) nucleic acids: identification of ligand, such as protein or RNA, binding sites;

d) catalytic polypeptides: polymers, preferably polypeptides, that are capable of promoting a chemical reaction involving the conversion of one or more reactants to one or more products; such polypeptides generally include a binding site specific for at least one reactant or reaction intermediate and an active functionality proximate to the binding site, in which the functionality is capable of chemically modifying the bound reactant [see, eg., U.S. Pat. No. 5,215,899];

e) hormone receptors: determination of the ligands that bind with high affinity to a receptor is useful in the development of hormone replacement therapies; for example, identification of ligands that bind to such receptors may lead to the development of drugs to control blood pressure; and

f) opiate receptors: determination of ligands that bind to the opiate receptors in the brain is useful in the development of less-addictive replacements for morphine and related drugs.

As used herein, antibody includes antibody fragments, such as Fab fragments, which are composed of a light chain and the variable region of a heavy chain.

As used herein, humanized antibodies refer to antibodies that are modified to include “human” sequences of amino acids so that administration to a human will not provoke an immune response. Methods for preparation of such antibodies are known. For example, the hybridoma that expresses the monoclonal antibody is altered by recombinant DNA techniques to express an antibody in which the amino acid composition of the non-variable regions is based on human antibodies. Computer programs have been designed to identify such regions.

As used herein, production by recombinant means by using recombinant DNA methods means the use of the well known methods of molecular biology for expressing proteins encoded by cloned DNA.

As used herein, substantially identical to a product means sufficiently similar so that the property of interest is sufficiently unchanged so that the substantially identical product can be used in place of the product.

As used herein, equivalent, when referring to two sequences of nucleic acids means that the two sequences in question encode the same sequence of amino acids or equivalent proteins. When “equivalent” is used in referring to two proteins or peptides, it means that the two proteins or peptides have substantially the same amino acid sequence with only conservative amino acid substitutions (see, eg., Table 1, above)that do not substantially alter the activity or function of the protein or peptide. When “equivalent” refers to a property, the property does not need to be present to the same extent [eg., two peptides can exhibit different rates of the same type of enzymatic activity], but the activities are preferably substantially the same. “Complementary,” when referring to two nucleotide sequences, means that the two sequences of nucleotides are capable of hybridizing, preferably with less than 25%, more preferably with less than 15%, even more preferably with less than 5%, most preferably with no mismatches between opposed nucleotides. Preferably the two molecules will hybridize under conditions of high stringency.

As used herein: stringency of hybridization in determining percentage mismatch is as follows:

1) high stringency: 0.1×SSPE, 0.1% SDS, 65° C.

2) medium stringency: 0.2×SSPE, 0.1% SDS, 50° C.

3) low stringency: 1.0×SSPE, 0.1% SDS, 50° C. It is understood that equivalent stringencies may be achieved using alternative buffers, salts and temperatures.

The term “substantially” identical or homologous or similar varies with the context as understood by those skilled in the relevant art and generally means at least 70%, preferably means at least 80%, more preferably at least 90%, and most preferably at least 95% identity.

As used herein, a composition refers to a any mixture. It may be a solution, a suspension, liquid, powder, a paste, aqueous, non-aqueous or any combination thereof.

As used herein, a combination refers to any association between two or among more items.

As used herein, fluid refers to any composition that can flow. Fluids thus encompass compositions that are in the form of semi-solids, pastes, solutions, aqueous mixtures, gels, lotions, creams and other such compositions.

As used herein, the abbreviations for any protective groups, amino acids and other compounds, are, unless indicated otherwise, in accord with their common usage, recognized abbreviations, or the IUPAC-IUB Commission on Biochemical Nomenclature (see, (1972) Biochem. 11:1726).

For clarity of disclosure, and not by way of limitation, the detailed description is divided into the subsections that follow. The description below is exemplified by reference to viral hemorrhagic diseases. It is understood that the methods, compositions, combinations and kits provided and described herein may be used for treatment of any disorder, disease or condition characterized by a deleterious immune response, particularly, but not limited to, those specificed herein. Such diseases, conditions and disorders include, but are not limited to: viral infections, such as viral hemorrhagic infections, lentivirus infections, HIV infections, herpes virus infections; bacterial infections, particularly infection with pathogenic strains of E. coli and Streptococcus; viruses associated with sleep disorders, such as HIV; parasitic infections, such as malaria; autoimmune diseases, such a thyroid diseases, rheumatoid arthritis, and lupis; sepsis; cachexia, such as the wasting associated with HIV infection and cancer; rheumatoid arthritis; chronic myelogenous leukemia and transplanted bone marrow-induced graft-versus-host disease; septic shock; immune complex-induced colitis; cerebrospinal fluid inflammation; endotoxemia; autoimmune disorders; multiple sclerosis; cell death associated with apoptosis; thyroid diseases and other endocrine disorders in which TNF or IL-1 is implicated or is a mediator; gynecological disorders, including endometriosis and infections associated therewith; and other diseases mediated by or associated with IL-1 and/or TNF. It is also understood that IL-1 and TNF expression serve as markers for these disorders and to monitor the treatments herein and the blood compositions herein, but that these inflammatory response compounds are not necessarily the only agents involved.

B. Combinations and Kits and Compostions for Treatment of Acute Inflammatory Responses

Combinations of therapeutic agents and also compositions for treatment of acute inflammatory responses are provided herein. Several embodiments are provided.

In one embodiment, blood-derived compositions, described below, are provided. These compositions are produced by contacting mammalian blood or a fraction thereof, in vitro or in vivo, with one or more tetracycline and/or tetracycline-like compounds, as defined herein, to induce a response that is assessed by monitoring the increase in level of TNF receptors and/or IL-1 receptors. The amount of compound contacted with the blood and time of contact is sufficient to induce at least a three-fold increase from baseline, which is variable from individual-to-individual and species-to-species, of TNF and/or IL-1 receptors. The total increase of either must be at least about three-fold to ensure a sufficient concentration of the receptors and other factors in the blood or fraction thereof. The resulting blood or fraction thereof can be further fractionated, such that the selected fraction retains the activity of the original blood, such as against hemorrhagic and inflammatory factors, and is then administered to a recipient mammal, that is preferably species and blood type matched to the donated blood or fraction. The blood or fraction thereof can be stored, preferably at about −70° C. or under other conditions appropriate for storage of blood products, but is preferably not freeze-dried.

The blood product may also be administered to the recipient in combination with a tetracycline and/or tetracycline-like compound. Such administration can be simultaneous or sequential. If administered separately they should be administered within 24 hours, preferably within 6 hours. When administered simultaneously they can be administered in a single composition, with the tetracycline and tetracycline-like compound(s) mixed in the blood-derived compositions. They blood-derived composition is preferably administered intravenously or intraperitoneally; the tetracycline and tetracycline-like compound is preferably administered orally. Multiple doses of each may be administered as needed. Precise dosage and regimen can be empirically determined.

The combination therapy may also include a known therapeutic treatment or regimen for a particular acute inflammatory disease, condition or disorder. Hence combinations of the blood-derived (or immune) compositions with tetracycline and/or tetracycline-like compounds are provided; combinations of the blood-derived (or immune) compositions with other therapeutic agents for treatment of a particular disorder, and combinations of the blood-derived (or immune) compositions with tetracycline and/or tetracycline-like compounds and with other therapeutic agents are provided. The component of combinations may be provided as separate compositions or may be provided as mixtures of two or more compositions. The tetracycline and tetracycline-like compounds are preferably administered orally and the blood-derived compositions are preferably administered by IV.

Kits containing the combinations are provided. The kits contain the components of the combinations, such as the blood-derived composition and tetracycline and/or tetracycline-like compounds, and optionally include instructions for administration to treat acute inflammatory response disorders. The reagents in the kits are packaged in standard pharmaceutical containers and packaging material. The kits may optionally contain additional components, such as syringes for administration of the compositions.

It is also shown herein that tetracycline and tetracycline-like compounds are effective for treatment of viral and bacterial infections, particularly, hemorrhagic fevers and infections with pathogenic E. coli. The tetracycline and tetracycline-like compounds may be administered with known treatments for hemorrhagic fevers. Combinations and kits containing the combinations of tetracycline and/or tetracycline-like compounds and such anti-hemorrhagic viral infections are also provided.

1. Tetracycline-like compounds

Tetracycline-like compounds, which include thalidomide, aureomycin and sulfa drugs, and any other compound that exhibits tetracycline-like activity, either in the ability to induce expression of TNF and/or IL-1 receptors in treated individuals, which can be determined in model animals as in the Examples below, or by the ability to alter folic acid metabolism in bacteria. Such compounds can be identified empirically. Any compounds that can do either are suitable for use in the methods of treating acute inflammatory responses provided herein.

2. Tetracycline compounds

a. Anti-inflammatory activity of tetracyclines

Tetracyclines are a well-known family of antibiotics that are active against a wide range of gram-positive and gram-negative bacteria. There are some indications in the art that tetracycline has anti-inflammatory activities, which are independent of its antibacterial activity (see, e.g., U.S. Pat. No. 5,773,430; U.S. Pat. No. 5,789,395; Shapira et al. (1996) Infect. Immun. 64:825-828; Kloppenburg et al. (1996) Antimicrob. Agents. Chemother. 40:934-940; Celerier et al. (1996) Arch. Dermatol. Res. 288:411-414; Milano et al. (1997) Antimicrob. Agents. Chemother. 41(1): 117-121; and U.S. Pat. No. 5,668,122). None, however, describe or suggest the use of tetracycline or tetracycline-like compounds for treatment of hemorrhagic fevers nor for production of blood-derived compositions for treatment of disorders, diseases and conditions characterized by or associated with an acute inflammatory response.

b. Exemplary tetracycline compounds

For purposes herein a tetracycline is any compound recognized by those of skill in the art to have the anti-inflammatory activities of a tetracycline and includes, all derivatives, including salts, esters and acids, analogs, prodrugs, modified forms thereof, and other compounds related to tetracycline as desribed above. The following are exemplary tetracycline compounds intended for use in the methods and compositions and combinations provided herein.

(1) Chlortetracycline

Chemically, chlortetracycline is 7-Chloro-4-dimethylamino-1,4,4a,5,5a,6,11,12a-octahydro-3,6,10,12,12a-pentahydroxy-6-methyl-1,11-dioxo-2-naphthacenecarboxamide. Chemical synonyms of chlortetracycline include 7-chloro-tetracycline, Acronize, Aureocina, Aureomycin, Biomitsin, Biomycin and Chrysomykine. For purposes herein, the name “chlortetracycline” is used herein, although all such chemical synonyms are contemplated. Chemical synonyms of chlortetracycline hydrochloride include, but are not limited to, Aureociclina and Isphamycin.

Chlortetracycline can be prepared according to methods known in the art. For example, chlortetracycline can be isolated from the substrate of Streptomyces aureofaciens (Duggar, Ann. N.Y. Acad. Sci. 51, 177 (1948); U.S. Pat. No. 2,482,055 (1949 to Am Cyanamid); and Broschard et al., Science 109, 199 (1949)). Purification of chlortetracycline is described in Winterbottom, etal., U.S. Pat. No. 2,899,422 (1959 to Am. Cyanamid). Other processes for preparation of chlortetracycline is described in U.S. Pat. Nos. 2,987,449 and 3,050,446.

(2) Demeclocycline

Chemically, demeclocycline is 7-Chloro-4-dimethylamino-1,4,4a,5,5a,6,11,12a-octahydro-3,6,10,12,12a-pentahydroxy-1,11-dioxo-2-naphthacenecarboxamide. Chemical synonyms of demeclocycline include 7-chloro-6-demethyltetracycline, demethylchlortetracycline (obsolete), RP 10192, Bioterciclin, Declomycin, Deganol, Ledermycin and Periciclina. For purposes herein, the name “demeclocycline” is used, although all such chemical synonyms are contemplated. Chemical synonyms of demeclocycline hydrochloride include, but are not limited to, Clortetrin, Demetraciclina, Detravis, Meciclin and Mexocine.

Demeclocycline can be prepared according to methods known in the art. For example, demeclocycline can be prepared according to the procedures described in McCormick et al., J. Am. Chem. Soc. 79, 4561 (1957); and U.S. Pat. No. 2,878,289 (1959 to Am. Cyanamid). Fermentation processes for demeclocycline preparation is described in U.S. Pat. Nos. 3,012,946, 3,019,172 and 3,050,446 (to Am. Cyanamid); Fr. pat. No. 1,344,645 (1963 to Merck & Co.); and Neidleman, U.S. Pat. No. 3,154,476 (1964 to Olin Mathieson). Demeclocycline hydrochloride is also available from Lederle Labs (Declomycin Tablets).

(3) Doxycycline

Chemically, doxycycline is 4-(Dimethylamino)-1,4,4a,5,5a,6,11,12a-octa-hydro-3,5,10,12,12a-pentahydroxy-6-methyl-1,11-dioxo-2-naphthacenecarboxamide monohydrate. Other chemical synonyms of doxycycline include: α-6-deoxy-5-hydroxytetracycline monohydrate; α-6-deoxyoxytetracycline monohydrate; or 5-hydroxy-α-6-deoxytetracycline monohydrateGS-3065; Azudoxat; Doxitard; Doxy-Puren; Investin; Liviatin; Nordox; Spanor; Vibramycin; and Vibravenös. For consistency, only the name “doxycycline” is used herein, although the all such chemical synonyms are contemplated.

Chemical synonyms of “doxycycline hydrochloride” include doxycycline hyclate, Diocimex, Doryx, Doxatet, Doxigalumicina, Doxy-II (caps), Doxylar, Doxy-Tablinen, Doxytem, duradoxal, Ecodox, Granudoxy, Hydramycin, Liomycin, Mespafin, Midoxin, Nivocilin, Novadox, Retens, Roximycin, Samecin, Sigadoxin, Tanamicin, Tecacin, Tetradox, Vibradox, Vibramycin Hyclate, Vibra-Tabs and Zadorin.

Doxycycline can be prepared according to methods known in the art. For example, 6-doxytetracyclines can be prepared according to the procedures described in Wittenau et al., J. Am. Chem. Soc. 84:2645 (1962); Stephens et al. J. Am. Chem. Soc. 85, 2643 (1963); Blackwood et al., U.S. Pat. No. 3,200,149 (1965 to Pfizer). Preparation, separation and configuration of 6α- and 6β-epimers are described in Wittenau et al., J. Am. Chem. Soc. 84, 2645 (1962); Stephens et al., ibid. 85, 2643 (1963).

Doxycycline calcium is available from Pfizer (Vibramycin Calcium Oral Suspension Syrup). Doxycycline hyclate is available from Pfizer (Vibramycin Hyclate Capsules; Vibramycin Hyclate Intravenous; Vibra-Tabs Film Coated Tablets), from Warner Chilcott Professional Products (Doryx Coated Pellets), from Warner Chilcott (Doxycycline Hyclate Capsules) and from Mylan (Doxycycline Hyclate Capsules and Tablets). Doxycycline monohydrate is available from Pfizer (Vibramycin Monohydrate for Oral Suspension) and from Oclassen (Monodox Capsules).

(4) Methacycline

Chemically, methacycline is [4S-(4α,4aα,5α,5aα,12aα)]-4-Di-methylamino-1,4,4a,5,5a,6,11,12a-octahydro-3,5,10,12,12a-pentahydroxy-6-methylene-1,11-dioxo-1-naphthacenecarboxamide. Chemical synonyms of methacycline include 6-methyleneoxytetracycline, 6-methylene-5-hydroxytetracycline, metacycline and Bialatan. For purposes herein, the name “methacycline” is used. It is understood that all chemical synonyms are contemplated. Chemical synonyms of methacycline hydrochloride include Andriamicina, Ciclobiotic, Germiciclin, Globociclina, Megamycine, Metadomus, Metilenbiotic, Londomycin, Optimycin, Physiomycine, Rindex and Rondomycin.

Demeclocycline can be prepared according to methods known in the art. For example, methacycline can be prepared from oxytetracycline (Blackwood et al., J. Am. Chem. Soc. 83 2773 (1961); 85, 3943 (1963); and Blackwood, U.S. Pat. No. 3,026,354 (1962 to Pfizer)).

(5) Minocycline

Chemically, minocycline is 4,7-Bis(dimethylamino)-1,4,4a,5,-5a,6,11,12a-octahydro-3,10,12,12a-tetrahydroxy-1,11-dioxo-2-naphthacenecarboxamide. Chemical synonyms of minocycline include 7-dimethylamino-6-demethyl-6-deoxytetracycline and Minocyn. For purposes herein, the name “minocycline” is used, but all such chemical synonyms are contemplated. Chemical synonyms of minocycline hydrochloride include Minocin, Klinomycin, Minomycin and Vectrin.

Minocycline can be prepared according to methods known in the art. For example, minocycline can be prepared according to the procedures described in Boothe, Petisi, U.S. Pat. Nos. 3,148,212 and 3,226,436 (1964 and 1965 to Am. Cyanamid). Synthesis of minocycline is described in Martell, Boothe, J. Med. Chem. 10, 44 (1967); Church etal., J. Org. Chem. 36, 723 (1971); and Bernardi et al., Farmaco Ed. Sci. 30, 736 (1975). Minocycline hydrochloride is available from Medicis (Dynacin Capsules), from Lederle Labs (Minocin Intravenous; Minocin Oral Suspension; and Minocin Pellet-Filled Capsules) and from Warner Chilcott Professional Products (Vectrin Capsules).

(6) Oxytetracycline

Chemically, oxytetracycline is 4-(Dimethylamino)-1,4,4a,5,-5a,6,11,12a-octahydro-3,5,6,10,12,12a-hexahydroxy-6-methyl-1,11-dioxo-2-naphthacenecarboxamide. Chemical synonyms of oxytetracycline include: glomycin; terr-fungine; riomitsin; hydroxytetracycline; Berkmycen; Biostat; Engemycin; Oxacycline; Oxatets; Oxydon; Oxy-Dumocyclin; Oxymycin; Oxypan; Oxytetracid; Ryomycin; Stevacin; Terraject; Terramycin; Tetramel; Tetran; Vendarcin; and Vendracin. For purposes herein, the name “oxytetracycline” is used, although all such chemical synonyms are contemplated. Chemical synonyms of oxytetracycline dihydrate include Abbocin, Clinimycin and Imperacin. Chemical synonyms of oxytetracycline hydrochloride dihydrate include Alamycin, Aquacycline, Arcospectron, Bio-Mycin, Duphacycline, Geomycin, Gynamousse, Macocyn, Macodyn, Occrycetin, Oxlopar, Oxybiocycline, Oxybiotic, Oxycycline, Oxyject, Oxylag, Stecsolin, Tetra-Tablinen and Toxinal.

Oxytetracycline can be prepared according to methods known in the art. For example, oxytetracycline can be isolated from the elaboration products of the antinomycete, Streptomyces rimosus, grown on a suitable medium (Finlay et al., Science 111, 85 (1950); Regna, Solomons, Ann. N.Y. Acad. Sci. 53, 221 (1950); Regna et al., J. Am. Chem. Soc. 73, 4211 (1951)), from Streptomyces rimosus (Sobin etal., U.S. Pat. No. 2,516,080 (1950 to Pfizer)), from S. xanthophaeus (Brockmann, Musso, Naturwiss. 41, 451 (1954); Brockmann et al., Ger. pat. 913,687 (1954 to Bayer), C.A. 53, 4662h (1959)). Total synthesis of the dl-form of oxytetracycline is described in H. Muxfeldt et al., ibid. 101, 689 (1979). Oxytetracycline hydrochloride is available from Pfizer (Terra-Cortril Ophthalmic Suspension; Terramycin with Polymyxin B Sulfate Ophthalmic Ointment; and Urobiotic-250 Capsules).

(7) Tetracycline

Chemically, tetracycline is 4-dimethylamino)-1,4,4a,5,5a,6-11,12a-octahydro-3,6,10,12,12a-pentahydroxy-6-methyl-1,11-dioxo-2-naphthacenecarboxamide. Chemical synonyms of tetracycline include deschlorobiomycin; tsiklomitsin; Abricycline; Achromycin; Agromicina; Ambramicina; Ambramycin; Bio-Tetra; Bristaciclina; Cefracycline suspension; Criseociclina; Cyclocmycin; Democracin; Hostacyclin; Omegamycin; Panmycin; Polycycline; Purocyclina; Sanclomycine; Steclin; Tetrabon; Tetracyn; Tetradecin. For purposes herein, the name “tetracycline”, although the all such chemical synonyms are contemplated.

Chemical synonyms (i.e. equivalents or generics) tetracycline hydrochloride, include Achro, Achromycin V, Ala Tet, Ambracyn, Artomycin, Cefracycline tablets, Cyclopar, Diacycline, Dumocyclin, Helvecyclin, Imex, Mephacyclin, Partrex, Quadracycline, Quatrex, Remicyclin, Ricycline, Ro-cycline, Stilciclina, Subamycin, Supramycin, Sustamycin, Tefilin, Teline, Telotrex, Tetrabakat, Tetrabid, Tetrablet, Tetrachel, Tetracompren, Tetra-D, Tetrakap, Tetralution, Tetramavan, Tetramycin, Tetrosol, Tetra-Wedel, Topicycline, Totomycin, Triphacyclin, Unicin, Unimycin and Vetquamycin-324. Chemical synonyms of tetracycline phosphate complex include Panmycin Phosphate, Sumycin, Tetradecin Novum, Tetrex and Upcyclin.

In addition to its ubiquitous commercial availability, tetracycline can be prepared according to methods known in the art. For example, tetracycline can be produced from Streptomyces spp. (Boothe et al. J. Am. Chem. Soc. 75, 4621 (1953); Conover et al., ibid. 4622; and Conover, U.S. Pat. No. 2,699,054 (1955)), from Streptomyces viridifaciens (Gourevitch, et al., U.S. U.S. Pat. Nos. 2,712,517; 2,886,595 (1955, 1959 to Bristol Labs)), from S. aureofaciens (U.S. Pat. Nos. 3,005,023; 3,019,173). Purification of tetracycline is described, for example, in U.S. Pat. No. 3,301,899. Preparation of tetracycline phosphate complex is described in Seiger, Weidenheimer, U.S. Pat. No. 3,053,892 (1962 to Am. Cyanamid). Total synthesis of tetracyclines is described in Boothe et al., J. Am. Chem. Soc. 81, 1006 (1959); Conover et al., ibid. 84, 3222 (1962). Tetracycline hydrochloride is available from Lederle Labs (Achromycin V Capsules), from Procter & Gamble Pharmaceutical (Helidac Therapy), from Lederle Standard (Tetracycline HCl Capsules) and from Mylan (Tetracycline Hydrochloride Capsules). Soluble tetracycline is preferred.

(8) Other Chemically-Modified Tetracyclines

Other tetracyclines include, but are not limited to, dedimethylaminotetracyclines, which include 4-dedimethylaminotetracycline, 4-dedimethylamino-5-oxytetracycline, 4-dedimethylamino-7-chlortetracycline, 4-hydroxy-4-dedimethyl-aminotetracycline, 5a, 6-anhydro-4hydroxy-4-dedimethylaminotetracycline, 6α-deoxy-5-hydroxy-4-dedimethylaminotetracycline, 6-demethyl-6-deoxy-4-dedimethylaminotetracycline, 4-dedimethylamino-12a-deoxytetracycline, 4-dedimethylamino-11-hydroxy-12a-deoxytetracycline, 12a-deoxy-4-deoxy-4-dedimethylaminotetracycline, 6α-deoxy-5-hydroxy-4-dedimethylaminodoxycycline, 12a,4a-anhydro-4-dedimethylaminotetracycline and minocycline-CMT i.e., 7-dimethylamino-6-demethyl-6-deoxy-4-dedimethylaminotetracycline. Further examples of chemically-modified tetracyclines contemplated for use herein, include but are not limited to, 6a-benzylthiomethylenetetracycline, the 2-nitrilo analogs of tetracycline (tetracyclinonitrile), the mono-N-alkylated amide of tetracycline, 6-fluoro-6-demethyltetracycline, 11a-chlortetracycline, tetracycline pyrazole and 12a-deoxytetracycline and its derivatives (see, e.g., U.S. Pat. No. 5,532,227).

Other chemically modified tetracyclines (CMT's) include, but are not limited to for example, 4-de(dimethylamino)tetracycline (CMT-1), tetracyclinonitrile (CMT-2), 6-demethyl-6-deoxy-4-de(dimethylamino)tetracycline (CMT-3), 7-chloro-4-de(dimethylamino)tetracycline (CMT-4), tetracycline pyrazole (CMT-5), 4-hydroxy-4-de(dimethylamino)tetracycline (CMT-6), 4-de(dimethylamino)-12.alpha.-deoxytetracycline (CMT-7), 6-deoxy-5.alpha.-hydroxy-4-de(dimethylamino)tetracycline (CMT-8), 4-de(dimethylamino)-12.alpha.-deoxyanhydrotetracycline (CMT-9) and 4-de(dimethylamino)minocycline (CMT-10) (see, e.g., U.S. Pat. No. 5,773,430). Further examples of tetracyclines modified for reduced antimicrobial activity include the 4-epimers of oxytetracycline and chlortetracycline (epi-oxytetracycline and epichlortetracycline).

Also contemplated and included are 4-dedimethylaminotetracyclines and the corresponding 5a,6-anhydro derivatives having an oxo, hydroxy, substituted imino, amino or substituted amino group other than dimethylamino at the C.multidot.4-position useful as antimicrobial agents. Examples of such 4-dedimethylaminotetracyclines derivatives include 5-Oxytetracycline, 7-Chlortetracycline, 6-Deoxy-5-oxytetracycline, 6-Deoxytetracycline, 6-Deoxy-6-demethyltetracycline, 7-Bromotetracycline, 6-Demethyl-7-chlortetracycline, 6-Demethyltetracycline, 6-Methylenetetracycline, 11a-Chloro-6-methylenetetracycline, 6-Methylene-5-oxytetracycline and 11a-Chloro-6-methylene-5-oxytetracycline (see, e.g., U.S. Pat. No. 4,066,694).

Aqueous solutions of chlortetracycline or salts thereof, a pharmaceutically acceptable calcium compound and 2-pyrrolidone as a co-solvent, where the solution has a pH of 8 to 10 is used as an injectable composition combining low viscosity, high potency, good clarity and good stability (see, U.S. Pat. No. 4,081,527).

Further, the tetracycline compounds and formulations that can be used herein include those compounds or formulations described in the following U.S. Pat. Nos. or those compounds or formulations that can be prepared according to the processes described in the following U.S. Pat. Nos.:

5,827,840 (Chemically-modified tetracyclines); 5,789,395 (Method of using tetracycline compounds for inhibition of endogenous nitric oxide production); 5,773,430 (Serine proteinase inhibitory activity by hydrophobic tetracycline); 5,770,588 (Non-antibacterial tetracycline compositions); 5,668,122 (Method to treat cancer with tetracyclines); 5,538,954 (Salts of tetracyclines); 5,532,227 (Tetracyclines including non-antimicrobial chemically-modified tetracyclines); 5,523,297 Non-antimicrobial tetracyclines); RE34,656 (Use of tetracycline to enhance bone protein synthesis and/or treatment of bone deficiency); 5,321,017 (Composition containing fluriprofen and effectively non-antibacterial tetracycline to reduce bone loss); 5,308,839 (Composition containing non-steroidal anti-inflammatory agent tenidap and effectively non-antibacterial tetracycline); 5,277,916 (Tetracycline dosage form); 5,258,372 (Tetracycline activity enhancement using doxycycline or sancycline); 5,250,442 (Method of treating rheumatoid arthritis using tetracycline); 5,223,248 (Non-antibacterial tetracycline compositions possessing antiplaque properties); 5,021,407 (Tetracycline activity enhancement); 4,935,412 (Non-antibacterial tetracycline compositions possessing anti-collagenolytic properties and methods of preparing and using same); 4,935,422 (Non-antibacterial tetracycline compositions possessing anti-collagenolytic properties and methods of preparing and using same); 4,925,833 (Use of tetracycline to enhance bone protein synthesis and/or treatment of osteoporosis); 4,837,030 (Novel controlled release formulations of tetracycline compounds); 4,704,383 (Non-antibacterial tetracycline compositions possessing anti-collagenolytic properties and methods of preparing and using same); 4,666,897 (Inhibition of mammalian collagenolytic enzymes by tetracyclines); 4,418,060 (Therapeutically active complexes of tetracyclines); 4,376,118 (Stable nonaqueous solution of tetracycline salt); 4,081,528 (Tetracycline compositions); 4,066,694 (4-Hydroxy-4-dedimethyl-amino-tetracyclines); 4,060,605 (Water-soluble derivative of 6-deoxy-tetracyclines); 3,993,694 (Tetracycline derivatives and process for preparing them); 3,983,173 (2-Carboxamido-substituted tetracyclines and process for their manufacture); 3,962,330 (Process for the preparation of 6-demethyl-6-deoxy-6-methylene-tetracyclines); 3,947,517 (Stereoselective introduction of tetracyclines hydroxyl group at 12(a) position in synthesis of tetracyclines); 5,387,703 (Process and intermediate for the purification of oxytetracycline); 5,075,295 (Novel oxytetracycline compositions); 4,829,057 (Oxytetracycline capsules with increased stability and methods of producing the same); 4,584,135 (Process for the preparation of an oxytetracycline-calcium silicate complex salt from fermentation broth); 4,399,127 (Injectable oxytetracycline compositions); 4,386,083 (Injectable oxytetracycline compositions); 4,259,331 (Oxytetracycline compositions); 4,020,162 (Oxytetracycline solution for parenteral, peroral and local administration and processes for the production thereof); 4,018,889 (Oxytetracycline compositions); 3,962,435 (Combination of oxytetracycline and 2,4-diamino-5-(3-alkoxy-4,5-methylenedioxybenzyl)pyrimidine); 3,962,131 (Rhodium containing catalyst and use thereof in preparation of 6-deoxy-5-oxytetracycline); 3,957,972 (Stable solutions of oxytetracycline suitable for parenteral and peroral administration and process of preparation); 5,258,372 (Tetracycline activity enhancement using doxycycline or sancycline); 4,086,332 (Doxycycline compositions); 4,061,676 (Recovery of doxycycline and products thereof); 3,957,980 (Doxycycline parenteral compositions); 3,932,490 (Doxycycline aceturate); 5,413,777 (Pulsatile once-a-day delivery systems for minocycline); 5,348,748 (Pulsatile once-a-day delivery systems for minocycline); 5,300,304 (Pulsatile once-a-day delivery systems for minocycline); 5,262,173 (Pulsatile once-a-day delivery systems for minocycline); and 4,701,320 (Composition stably containing minocycline for treating periodontal diseases).

Hence tetracycline compounds are well known to those of skill in the art; and tetracycline-like compounds can be readily identified.

C. Hemorrhagic Viruses and the Immune Response

The immune response to hemorrhagic viral infection appears to follow the a scheme that includes: viral activation of macrophages, T and B lymphocytes; production of mediators by mononuclear cells, including cytokines such as, interleukin (IL)-1 and IL-2, interferon (IFN), and/or tumor necrosis factor (TNF); changes of the proliferative activity of the cells; alterations of lymphocyte subpopulations (CD4 and CD8); and propagation of virus in immunocompetent cells.

A decrease of lymphocyte proliferative activity in response to mitogen stimulation, a decrease of the number of T and B lymphocytes, and an inversion of CD4\CD8 lymphocyte ratios (Fisher-Hoch etal. (1987) J. Infect. Dis., 155:465-474; Vallejos et al. (1985) Medicina (Buenos-Aries), 45:407; Enria et al. (1986) Med. Microbiol. Immunol, 175:173-176) have been demonstrated in arenaviral hemorrhagic fevers.

Clinical observations and experimental study of these fevers has demonstrated a marked production of the inflammatory cytokines, such as TNF, IL-1, IFN, during these diseases. Pronounced production of serum IFN was seen during experimental infection of guinea pigs and monkeys with Marburg and Ebola viruses with lethal outcome (Ognatyev et al., Voprosy Virusologii, 39:13-17 (1994); Ignatyev et al., Voprosy Virusologii, 40:109-113 (1995); lgnatyev et al., J. Biotechnol, 44:111-118 (1996)). The infection of human macrophages with Marburg virus leads to increased release of TNF-α, which is one of several cytokines typically secreted by macrophages (Feldmann et al., J. Virol., 70:2208-2214 (1996)). Infection of monkeys with Ebola virus was also accompanied by increased serum TNF-α levels (Ignatyev, Curr. Top. Microbiol. Immunol., 235:205-217 (1999)).

Increased levels of TNFα and IFN-α in patients with Argentine hemorrhagic fever correlate with the severity of disease; whereas IL-1β levels in patients do not differ from those in normal controls (see, Heller et al., J. Infect. Dis., 166:1203 (1992)). Increased production of nitric oxide (NO) in patients with hemorrhagic fever with renal syndrome has been reported (Linderholm et al., Infection, 24:337-340 (1996)).

Similarly high concentrations of IL-1 and TNF during the development of the human septic shock are known to contribute to lethal outcome (see, Calandra et al., J. Infectious Diseases, 161:982-987 (1990); Cannon et al., J. Infectious Diseases, 161 :79-84 (1990)).

Defective humoral responses and extensive intravascular apoptosis are associated with fatal outcome in ebola virus-infected patients (Baize et al., Nature Medicine, 5(4):423-426 (1999)). In survivors, early and increasing levels of IgG, directly against mainly against the nucleoprotein and the 40-kDa viral protein, were followed by clearance of circulating viral antigen and activation of cytotoxic T cells. In contrast, fatal infection was characterized by impaired humoral responses, with absent specific IgG and barely detectable IgM.

The compositions and method provided herein provide a means to treat infections with hemorrhagic viruses. In particular, the blood-derived compositions, which can be readily produced by contacting blood from a donor in vitro or in vivo with a compound such as as, a tetracycline or tetracycline-like compound, and then harvesting, preferably serum or plasma, which can be infused into the mammal with the infection, is effective for treatment. The response in the donor blood or fraction thereof can be observed as quickly as six hours after administration of the tetracycline and tetracycline-like compound or contacting with the blood. The infected mammal can also be treated with a tetracycline and tetracycline-like compounds prior to administration of the blood-derived composition, simultaneously and/or subsequently. Additional anti-hemorrhagic viral treatments and agents may also be administered.

D. Pharmaceutical Compositions, Formulation and Modes of Administration Thereof

Blood-derived compositions for administration, preferably for systemic administration, for treatment of acute inflammatory responses are provided. These are preferably provided in a form for systemic, such as intraperitoneal or intravenous administration. They may be concentrated or diluted by standard methods; preferably they are not subjected to freeze-drying.

Combinations of the blood-derived compositions with tetracycline and/or tetracycline-like compounds are also provided. These combinations may be packaged as kits and are intended for treatment of the acute inflammatory responses.

Also provided for treatment of the viral hemorrhagic diseases and also bacterial infections, such as E. coli, are tetracycline and tetracycline-like compounds, and also combinations of a composition containing one or more tetracycline compoundis) and a composition containing an anti-viral-hemorrhagic agent, preferably in a pharmaceutically acceptable carrier or excipient. The tetracycline compound(s) and anti-viral-hemorrhagic agent are packaged as separate compositions for administration together or sequentially or intermittently. Alternatively, they can be contained in a single composition for administration as a single composition. The combinations can be packaged as kits.

In a preferred embodiment, a composition suitable for oral delivery, includes one or more tetracycline compounds and an anti-viral-hemorrhagic agent, and a pharmaceutically acceptable carrier or excipient in tablet, capsule, or other single unit dosage form is provided.

Any tetracycline and tetracycline-like compound(s), including those described herein, when used alone or in combination with other compounds, that can alleviate, reduce, ameliorate, prevent, or place or maintain in a state of remission of clinical symptoms or diagnostic markers associated with acute inflammatory responses, such as viral hemorrhagic diseases or disorders, particularly those viral hemorrhagic diseases or disorders caused by infection of a Bunyaviridae, a Filoviridae, a Flaviviridae, or an Arenaviridae virus, can be used in the present combinations.

Suitable anti-viral hemorrhagic agents are described in the following section.

1. Anti-viral hemorrhagic agents

The tetracycline and tetracycline-like compounds and the blood-derived compositions provided herein can be administered alone or in combination with other agents, such as IL-1 inhibitors and/or TNF inhibitors, appropriate vaccines and other drugs for treatment of acute inflammatory diseases and disorders.

a. Interleukin-1 (IL-1) inhibitors

Any IL-1 inhibitor that prevents or decreases production, post-translational modification(s), maturation, or release of IL-1, or any substances that interfere with or decrease the efficacy of the interaction between IL-1 and IL-1 receptor is contemplated for use in combination with the tetracycline and tetracycline-like compounds and/or the blood-derived compositions. Preferably, the IL-1 inhibitor is an anti-IL-1 antibody, an anti-IL-1 receptor antibody, an IL-1 receptor antagonist, an IL-1 production inhibitor, an IL-1 receptor production inhibitor and an IL-1 releasing inhibitor.

Monoclonal antibodies, particurlarly humanized antibodies are preferrred. Anti-IL-1 antibodies are known (see, e.g., U.S. Pat. Nos. 4,772,685 and 4,994,553). Anti-IL-1 receptor antibodies are also known (see, e.g., Chen et al., Cancer Res., 58(16): 3668-76 (1998); Clark et al., J. Interferon Cytokine Res., 16(12): 1079-88 (1996); Zerek-Melen et al., Eur. J. Endocrinol., 131(5): 531-4 (1994); Mclntyreet al. (1991) J. Exp. Med., 173(4):931-9; Benjamin et al. (1990) Prog. Clin. Biol. Res., 349:355-6) are used.

An IL-1 receptor antagonist can be an IL-1 receptor antagonist (IL-1Ra; see, e.g., SEQ ID No. 5; see, also U.S. Pat. Nos. 5,863,769, 5,837,495, 5,739,282, 5,508,262, 5,455,330, 5,334,380, Bendele et al., Arthritis Rheum., 42(3):498-506 (1999); Kuster et al., Lancet, 352(9136):1271-7 (1998); Bendele et al., J. Lab. Clin. Med., 125(4): 493-500 (1995); and Wetzler et al., Blood, 84(9):3142-7 (1994)), an IL-1 receptor intracellular ligand protein, a Type II IL-1 receptor, a soluble IL-1 receptor, a non-functional mutein of IL-1, a non-functional mutein of IL-1 receptor or a small molecule antagonist.

IL-1 receptor intracellular ligand proteins (see, e.g., SEQ ID Nos. 6, 7, 8 and 9; see also U.S. Pat. No. 5,817,476), such as type 11 IL-1 receptor (see, e.g., SEQ ID No. 4; see, also U.S. Pat. Nos. 5,464,937 and 5,350,683) or soluble IL-1 receptors (see, e.g., U.S. Pat. Nos. 5,767,064, RE35,450, 5,492,888, 5,488,032, 5,319,071 and 5,180,812) are contemplated. Soluble receptors contain residues 1-312, 1-314, 1-315, 1-316, 1-317, 1-318 and 1-319 of the full-length receptor for which sequence is set forth in SEQ ID No. 3 or 4). Non-functional muteins of IL-1 (see, e.g., U.S. Pat. No. 5,286,847) can be used (e.g., in which the Arg residue at position 127 of the precursor IL-1β protein sequence (see, SEQ ID No. 2) is replaced with gly). The small molecule IL-1 receptor antagonist can be a histamine antagonist (see, e.g., U.S. Pat. No. 5,658,581), an aryl-or heteroaryl-1-alkyl-pyrrole-2-carboxylic acid compound (see, e.g., U.S. Pat. Nos. 5,039,695 and 5,041,554) or a 5-lipoxygenase pathway inhibitor (U.S. Pat. No. 4,794,114).

The IL-1 inhibitor can be an IL-1 production inhibitor, such as an antisense oligonucleotide (see, e.g., Yahata et al., Antisense Nucleic Acid Drug Dev., 6(1):55-61 (1996); Fujiwara et al., Cancer Res., 52(18): 4954-9 (1992); see, also SEQ ID. No. 10, which sets forth an exemplary anti-sense oligonucleotide specific for IL-1,6; and Maier et al., Science, 249:1570-4 (1990); SEQ ID No. 11, which sets forth an exemplary antisense oligonucleotide specific for IL-1 α) can be used.

The IL-1 production inhibitor can be a small molecule inhibitor, such as 5-hydroxy and 5-methoxy 2-amino-pyrimidine (see, e.g., U.S. Pat. No. 5,071,852), 3-substituted-2-oxindole-1-carboxamide (see, e.g., U.S. Pat. Nos. 4,861,794 and 5,192,790), 4,5-diaryl-2(substituted)imidazole (see, e.g., U.S. Pat. No. 4,780,470) and 2-2′-[1,3-propan-2-onediyl-bis(thio)]bis-1-H-imidazole (see, e.g., U.S. Pat. No. 4,778,806).

The IL-1 inhibitor can be an IL-1 receptor production inhibitor, such as an antisense oligonucleotide (see, e.g., SEQ ID No. 12, which provides an antisense oligonucleotide designated ISIS 8807; see, also Miraglia et al., Int. J. Immunopharmacol., 18(4):227-40 (1996); the oligonucleotide set forth in SEQ ID No. 13; and Burch et al., J. Clin. Invest., 88(4):1190-6 (1991)) can be used.

The IL-1 inhibitor can be an IL-1 releasing inhibitor, such as an IL-1 converting enzyme inhibitor e.g., N-substituted glutamic acid derivative (see, U.S. Pat. No. 5,744,451), y-pyrone-3-acetic acid (U.S. Pat. No. 5,411,985), probucol (U.S. Pat. No. 4,975,467), disulfiram, tetrakis [3-(2,6-di-tert-butyl-4-hydroxyphenyl)propionyloxy methyl]methane or 2,4-di-isobutyl-6-(N,N-dimethylaminomethyl)-phenol (U.S. Pat. No. 5,034,412), a peptide based interleukin-1 beta converting enzyme (ICE) inhibitor (Okamoto et al., Chem. Pharm. Bull. (Tokyo) 47(1):11-21 (1997)), a pyridazinodiazepine (Dolle et al., J. Med. Chem., 40(13):1941-6 (1997)), SDZ 224-015 (Elford et al., Br. J. Pharmacol., 115(4):601-6 (1995)), an aspartate-based inhibitor (Mashima et al., Biochem. Biophys. Res. Commun., 209(3):905-15 (1995)), an aspartyl alpha-((1-phenyl-3-(trifluoromethyl)-pyrazol-5-yl)oxy)methyl ketone (Dolle et al., J. Med. Chem., 37(23):3863-6 (1994)), L-741,494 (Salvatore et al., J. Nat. Prod., 57(6):755-60 (1994); see U.S. Pat. No. 5,843,904), TX (see U.S. Pat. No. 6,020,477), CPP-32 and CMH-1 (Margolin et al., J. Biol. Chem., 272(11):7223-8 (1997)), a peptide inhibitor of ICE, YVAD-CHO (de Bilbao et al., Neuroreport, 7(18):3051-4 (1996)), benzyloxycarbonyl-valinylaianylaspartylfluoromethyl ketone (Cain et al., J. Biochem., 314(Pt 1):27-32 (1996)) bocaspartyl (benzyl) chloromethylketone (BACMK) (Estrov et al., Blood, 86(12):4594-602 (1995)) and L-709,049 (Fletcher et al., J. Interferon Cytokine Res., 5(3):243-8 (1995)).

Other IL-1 inhibitors may also be used (see, e.g., U.S. Pat. No. 5,804,599 (Interleukin-1 production inhibiting compound), U.S. Pat. No. 5,453,490 (Recombinant human interleukin-1 inhibitors), U.S. Pat. No. 5,334,380 (Anti-endotoxin, interleukin-1 receptor antagonist), U.S. Pat. No. 5,075,222 (Interleukin-1 inhibitors), U.S. Pat. No. 5,034,412 (Interleukin-1 release inhibitors), U.S. Pat. No. 5,011,857 (Interleukin-1 release inhibitors), U.S. Pat. No. 4,975,467 (Interleukin-1 release inhibitors), U.S. Pat. No. 4,870,101 (Interleukin-1 release inhibitors) and Ray et al., Cell, 69(4):597-604 (1992) (Cowpox virus encoded interleukin-1 beta converting enzyme inhibitor).

b. Tumor necrosis factor (TNF) inhibitors

TNF inhibitors may also be used. These may be used in place of or in addition to IL-1 inhibitors. Any inhibitor of TNF activity is contemplated for use herein. Among the preferred inhibitors are anti-TNF antibodies, anti-TNF receptor antibodies, TNF receptor antagonists, TNF production inhibitors, TNF receptor production inhibitors and a TNF releasing inhibitors.

The anti-TNF antibody or the anti-TNF receptor antibody can be a monoclonal antibody, which is preferably, humanized. Such antibodies are known (e.g., the anti-TNF antibodies Mabp55r and Mabp75r (Tanaka et al., Neurol. Med. Chir. (Tokyo), 38(12):812-818 (1998)), 3B10 and h3B10-9 (Nagahira et al., J. Immunol. Methods., 222(1-2):83-92 (1999)), MAK 195F (Holler et al., Blood., 86(3):890-0 (1995)), CA2 (Centocor, Inc., Malvern, Pa.; Elliott et al., Lancet, 344:1125-1127 (1994); Cope et al., J. Clin. Invest., 94:749-760 (1994)) and CDP571 (Rankin et al., Br. J. Rheumatol., 34(4):334-342 (1995); U.S. Pat. Nos. 5,741,488, 5,698,195, 5,654,407, 5,626,321, 5,656,272, 5,436,154, 5,360,716, 5,231,024 and 5,795,967; and Cargile et al., Am. J. Vet. Res., 56(11):1451-9 (1995)).

The TNF receptor antagonist can be a purified soluble TNF receptor, a non-functional mutein of TNF receptor, a non-functional mutein of TNF and a small molecule antagonist. Non-functional muteins of TNF receptor are known (see, e.g., U.S. Pat. Nos. 5,863,786, 5,773,582, 5,606,023, 5,597,899, 5,519,119, 5,486,463, 5,422,104, 5,247,070 and 5,028,420). Small molecule antagonists, such as a mercapto alkyl peptidyl compound (see, e.g., U.S. Pat. No. 5,872,146), an arylsulfonyl hydroxamic acid derivative (U.S. Pat. No. 5,861,510), a salt of an alkaline-earth metal (U.S. Pat. No. 5,851,556), a pentoxifylline (U.S. Pat. No. 5,763,446), a hydroxamic acid compound (U.S. Pat. No. 5,703,092), a retinoic acid (U.S. Pat. No. 5,658,949), a histamine antagonist (U.S. Pat. No. 5,658,581), a leflunomide (U.S. Pat. No. 5,547,970), a 1-Alkoxy-2-(alkoxy- or cycloalkoxy-)-4-(cyclothioalkyl- or cyclothioalkenyl-) benzene (U.S. Pat. No. 5,541,219), a vinigrol (U.S. Pat. No. 5,306,732), a cyclohexene-ylidene derivative (U.S. Pat. No. 5,605,923), a quinazoline compound (U.S. Pat. No. 5,646,154) and BN 50739 (Rabinovici et al., J. Pharmacol. Exp. Ther., 255(1):256-63 (1990)) are also contemplated for use herein in combination with the tetracycline and tetracycline-like compounds and/or blood-derived compositions.

The TNF receptor antagonist can be a TNF receptor death domain ligand protein, a tumor necrosis factor binding protein (TNF-BP), a TNF receptor-IgG heavy chain chimeric protein (Peppel et al., J. Exp. Med., 174(6):1483-9 (1991)), a bacterial lipopolysaccharide binding peptide derived from CAP37 protein (U.S. Pat. No. 5,877,151) and a Myxoma virus T2 protein (Schreiber et al., J. Biol. Chem., 271(23):13333-41 (1996)). Exemplary TNF receptor death domain ligand proteins include those described in U.S. Pat. Nos. 5,849,501, 5,847,099, 5,843,675, 5,852,173 and 5,712,381 are used (see, also SEQ ID Nos. 18, 19, 20 and 21). Also, the TNF-BPs described in U.S. Pat. No. 5,811,261, which describes TBP-1 a 180 amino acid protein isolated from human urine, U.S. Pat. Nos. 5,808,029, 5,776,895, 5,750,503, which describe chimeric TNF-BPs containing the soluble portion of the P55 TNF receptor and all but the first domain of the constant region of IgG1 or IgG3 heavy chains, and the TNF-BPs described in Colagiovanni et al., Immunopharmacol. Immunotoxicol., 18(3):397-419 (1996) and Olsson et al., Biotherapy., 3(2):159-65 (1991), which describes a 50 kD protein isolated from human urine, can be used.

The TNF inhibitor can be an TNF production inhibitor, such as an antisense oligonucleotide (see, e.g., SEQ ID No. 22; see, also U.S. Pat. No. 5,705,389). Other TNF production inhibitors are known (see, e.g., U.S. Pat. No. 5,776,947 (quinoline-3-carboxamide compounds), U.S. Pat. No. 5,691,382 (matrix metaloproteinase inhibitors), U.S. Pat. No. 5,648,359, U.S. Pat. No. 5,616,490 (ribozymes targeted to TNFα RNA), U.S. Pat. Nos. 5,304,634, 5,420,154 and 5,547,979 (derivatives of 2-pyrrolidinones)).

TNF receptor production inhibitor include antisense oligonucleotides. The TNF inhibitor can be a TNF releasing inhibitor (see, e.g., U.S. Pat. No. 5,869,511 (isoxazoline compounds), U.S. Pat. No. 5,563,143 (catechol diether compounds), and U.S. Pat. No. 5,629,285 (peptidyl derivatives having active groups capable of inhibiting TACE such as, hydroxamates, thiols, phosphoryls and carboxyls)

Other TNF inhibitors are contemplated (see, e.g., U.S. Pat. No. 5,886,010 (TNFα inhibitors), U.S. Pat. No. 5,753,628 (peptide inhibitors of TNF containing predominantly D-amino acids), U.S. Pat. No. 5,695,953 (DNA that encodes a tumor necrosis factor inhibitory protein), U.S. Pat. No. 5,672,347 (tumor necrosis factor antagonists), U.S. Pat. No. 5,582,998 (monoclonal antibodies against human TNF-BP I), U.S. Pat. No. 5,478,925 (multimers of the soluble forms of TNF receptors), U.S. Pat. No. 5,464,938 (isolated viral protein TNF antagonists), U.S. Pat. No. 5,359,039 (isolated poxvirus A53R-equivalent tumor necrosis factor antagonists), U.S. Pat. No. 5,136,021 (TNF-inhibitory protein), U.S. Pat. No. 5,118,500 (xanthine derivatives), U.S. Pat. No. 5,519,000 (peptides that include 4-25 amino acids and bind to tumor necrosis factor-α) and U.S. Pat. No. 5,641,751.

C. Anti-viral vaccine, antibody and virally-activated immune cells and serum

For treatment of viral infections, particularly hemorrhagic fever infections, the tetracycline or tetracycline-like compounds and/or blood-derived composition may be administered in combination with an anti-viral vaccine, antibody and/or virally activated immune cells or serum.

Any anti-viral vaccines, anti-viral antibodies, viral-activated immune cells and viral-activated immune serums, when used alone or in combination with other compounds, that can alleviate, reduce, ameliorate, prevent, or place or maintain in a state of remission of clinical symptoms or diagnostic markers associated with viral hemorrhagic diseases or disorders, particularly those viral hemorrhagic diseases or disorders caused by infection of a Bunyaviridae, a Filoviridae, a Flaviviridae, or an Arenaviridae virus, can be used in the present combinations and in the methods of treatment in combination with administration of a tetracycline compound. Exemplary anti-viral treatments are agents include but are not limited to the following.

(1) Anti-viral vaccine

Anti-viral vaccines can be prepared according to the methods known in the art (see Current Protocols in Immunology (Ed. Coligan et al.) John Wiley & Sons, Inc., 1997). Any types of vaccines, including attenuated viruses, protein or peptide vaccines or nucleotide vaccines can be used.

(a) Anti-Bunyaviridae Vaccine

An anti-Bunyaviridae vaccine, preferably, an anti-Hantaan virus vaccine (see, e.g., U.S. Pat. No. 5,298,423 (nucleotide sequences coding for Hantaan virus nucleocapsid protein and glycoproteins G1 and G2), U.S. Pat. No. 5,183,658 (the purified and inactivated Hantaan virus ROK84/105), Chu, et al., J. ViroL, 69(10):6417-23 (1995) (a vaccinia virus-vectored vaccine expressing the M and the S segments of Hantaan (HTN) virus)) can be used.

(b) Anti-Filoviridae Vaccine

An anti-Filoviridae vaccine, such as an anti-ebola virus vaccine is used (e.g., the vaccines described in Chupurnov, et al., Vopr. Virusol., 40(6):257-60 (1995) (inactivated viral agents (Nonlethal strain of the virus)), Lupton, et al., Lancet, 2(8207):1294-5 (1980) (inactivated vaccine) and Sergeev, et al., Vopr. Virusol., 42(5):226-9 (1997) (immunomodifiers ridostin, reaferon, and polyribonate)) are used.

In another embodiment, an anti-Marburg virus vaccine is used (e.g., the vaccines described in Hevey, et al., Virology, 239(1):206-16 (1997) (Baculovirus recombinants were made to express the MBGV glycoprotein (GP) either as a full-length, cell-associated molecule or a slightly truncated (5.4%) product secreted into medium; and killed (irradiated) MBGV antigen)) can be used.

(c) Anti-Flaviviridae Vaccine

An anti-Flaviviridae vaccine, such as an anti-Dengue virus vaccine, can be used (e.g., U.S. Pat. No. 5,494,671, Becker, Virus Genes, 9(1):33-45 (1994) (Dengue fever virus and Japanse encephalitis virus synthetic peptides with motifs to fit HLA class I haplotypes), Blok, et al., Virology., 187(2):573-90 (1992) (Dengue-2 virus vaccine), Dharakul, et al., J. Infect. Dis., 170(1):27-33 (1994) (live attenuated Dengue virus type 2 vaccine), Green, et al., J. Virol., 67(10):5962-7 (1993) (live attenuated Dengue virus type 1 vaccine), Hoke, et al., Am. J. Trop. Med. Hyg., 43(2):219-26 (1990) (attenuated Dengue 4 (341750 Carib) virus vaccine), Khin, et al., Am. J. Trop. Med. Hyg., 51(6):864-9 (1994), (Dengue-2 PDK53 candidate vaccine), Kinney, et al., Virology., 230(2):300-8 (1997) (attenuated vaccine derivative, strain PDK-53), Leblois, et al., Nucleic Acids Res., 21(7):1668 (1993) (Dengue virus type 2 (strain PR-159) NS1 gene and its vaccine derivative), Marchette, et al., Am. J. Trop. Med. Hyg., 43(2):212-8 (1990) (attenuated Dengue 4 (341750 Carib) virus vaccine), Price, et al., Am. J. Epidemiol., 94(6):598-607 (1971) (injection with Dengue virus), Putnak, et al., Am. J. Trop. Med. Hyg., 55(5):504-10 (1996) (purified, inactivated, Dengue-2 virus vaccine prototype made in fetal rhesus lung cells), Putnak, et al., J. Infect. Dis., 174(6):1176-84(1996) (purified, inactivated, Dengue-2 virus vaccine prototype in Vero cells), Schlesinger, et al., J Gen Virol., 68(3):853-7 (1987) (Dengue 2 virus non-structural glycoprotein NS1)).

(d) Anti-Arenaviridae Vaccine

Anti-Arenaviridae vaccine such as, an anti-Junin virus vaccine (e.g., vaccines described in Boxaca, et al., Medicina (BAires), 41(4):25-34 (1981) (Variant XJO of Junin virus), Contigiani, et al., Acta Virol., 37(1):41-6 (1993) (Candid 1 attenuated strain of Junin virus), Coto, et al., J Infect Dis., 141(3):389-93 (1980) (Protection of guinea pigs inoculated with Tacaribe virus against lethal doses of Junin virus), de Guerrero, et al., Acta Virol., 29(4):334-7 (1985) (attenuated XJO Junin virus (JV) strain), Ghiringhelli, et al., Am J Trop Med Hyg., 56(2):216-25 (1997) (Junin virus vaccine strain (Candid #1), Remesar, et al., Rev Argent Microbiol., 21(3-4):120-6 (1989) (the attenuated XJC13 Junin virus strain), Samoilovich, et al., Am J Trop Med Hyg., 32(4):825-8 (1983) (attenuated XJC13 strain of Junin virus), Videla, et al., J Med Virol., 29(3):215-20 (1989) (Formalin inactivated Junin virus: The XJ-Clone 3 strain of Junin virus) and Weissenbacher, et al., Intervirology., 6(1):42-9 (1975-76) (Tacaribe virus)) can be used.

An anti-Lassa vaccine can be used (e.g., vaccines described in Auperin, et al., Virus Res., 9(2-3):233-48 (1988) (a recombinant vaccinia virus expressing the Lassa virus glycoprotein gene), Fisher-Hoch, et al., Proc Natl Acad Sci USA, 86(1):317-21 (1989) (a recombinant vaccinia virus expressing the Lassa virus glycoprotein gene), Kiley, et al., Lancet, 2(8145):738 (1979) (Immunization with closely related Arenavirus), Morrison, et al., Virology, 171 (1):1179-88 (1989) (Vaccinia virus recombinants expressing the nucleoprotein or the envelope glycoproteins of Lassa virus)).

An anti-Machupo virus vaccine (see, e.g., Eddy, et al., Bull World Health Organ., 52(4-6):723-7 (1975)) can be used.

(2) Anti-viral antibodies

Anti-viral antibodies can be prepared according to the methods known in the art (see Current Protocols in Immunology (Ed. Coligan et al.) John Wiley & Sons, Inc., 1997). Any types of antibodies, including polyclonal, monoclonal, humanized, Fab fragment, (Fab)₂ fragment and Fc fragment, can be used. In a specific embodiment, a monoclonal anti-viral antibody is used. Preferably, the monoclonal antibody is humanized. Also preferably, an IgG or IgM anti-viral antibody is used.

(a) Anti-Bunyaviridae Antibody

An anti-Bunyaviridae antibody, such as an anti-Hantaan virus antibody can be used (see, e.g., Kikuchi, et al., Arch. Virol., 143(1):73-83 (1998) (Neutralizing monoclonal antibody (MAb) to envelope protein G1 (16D2) and G2 (11E10)), Liang, et al., Virology, 217(1):262-71 (1996) (MAb to G2(HCO2)).

(b) Anti-Filoviridae Antibody

An anti-Filoviridae antibody, such as an anti-ebola virus antibody can be used (see, e.g., the following Genbank accession numbers for suitable antigenic proteins: 1EBOA-1EBOF, AAD14582-AAD14590, AAC57989-AAC57993, AAC54882-AAC54891, AAC24345-AAC24346, AAC09342, CAA47483, AAB81001-AAB81007, S23155, VHIWEB, S32584-S32585, AAB37092-AAB37097, AAA96744-AAA96745, AAA79970, CAA43578-CAA43579 and AAA42976-AAA42977, and for nucleic acids: AF086833, U77384-U77385, U8116-U23417, U23187, U23152, U23069,AF034645, AF054908, X67110, L11365, U28077, U28134, U28006, U31033, U23458, X61274, J04337 and M33062).

An anti-Marburg antibody can be used. The antibodies can be raised against Marburg virus protein sequences with the following Genbank accession numbers are used: AAC40455-AAC40460, VHIWMV, RRIWMV, S44052-S44053, S33316, S32582-S32583, A45705, B45705, S44049, S44054, CAA78114-CAA78120, CAA82536-CAA82542, CAA45746-CAA45749, CAA48507-CAA48509 and AAA46562-AAA46563 or encoded by nucleic acid molecules containing nucleotide sequences with the following Genbank accession numbers are used: AF005730-AF005735, Z12132, Z29337, X64405-X64406, X68493-X68495, M72714, M92834 and M36065.

(c) Anti-Flaviviridae Antibody

An anti-Flaviviridae antibody,. such as an anti-Dengue virus antibody is used (see, e.g., Bhoopat, et al., Asian Pac. J. Allergy Immunol., 14(2):107-13 (1996), Hiramatsu, et al., Virology., 224(2):437-45 (1996) (mAb3H5), Roehrig, et al., Virology, 246(2):317-28 (1998) (Murine monoclonal antibodies (MAbs) specific for the envelope (E) glycoprotein of DEN 2 virus: Domains A and B), Tadano, et al., J. Gen. Virol., 70 (6):1409-15 (1989) (MAb against the DEN-4 virus core protein Mr 15.5K), Trirawatanapong, et al., Gene, 116(2):139-50 (1992) (mAb3H5)).

(d) Anti-Arenaviridae Antibody

An anti-Arenaviridae antibody, such as an anti-Junin virus antibody can be used (see, e.g., the antibodies described in Mackenzie, et al., Am. J. Trop. Med. Hyg., 14(6):1079-84 (1965)).

An anti-Lassa antibody can be used (see, e.g., the antibodies described in Kunitskaia, et al., Zh Mikrobiol Epidemiol Immunobiol., 3:67-70 (1991) and Schmitz, et al., Med. Microbiol. Immunol. (Berl)., 175(2-3):181-2 (1986)).

An anti-Machupo antibody can be used (see, e.g., Mackenzie, et al., Am. J. Trop. Med. Hyg., 14(6):1079-84 (1965)).

(3) Viral-activated immune cell and serum

Viral-activated immune cells and sera can be prepared according to the methods known in the art (see Current Protocols in Immunology (Ed. Coligan et al.) John Wiley & Sons, Inc., 1997). Among the cells that can be used for treatment are virally-activated cytotoxic cells (see, Asada, et al., J. Gen. Virol., 68(7):1961-9 (1987) (Adoptive transfer of immune serum or immune T cells for treating Hantaan virus); Nakamura, et al., J. Infect. Dis., 151(4):691-7 (1985) (Immune spleen cells for treating Hantaan virus); Jahrling, et al., J. Infect. Dis., 179(Suppl1):S224-34 (1999) (Hyperimmune equine IgG for treating ebola virus); Mupapa, et al., J. Infect. Dis., 179(Suppl1):S18-23 (1999) (Blood transfusions with blood donated by convalescent patients for treating ebola virus), Avila, et al., J. Med. Virol., 21(1):67-74 (1987) (Immune serum treatment of Junin virus infection), Blejer, et al., Intervirology., 21(3):174-7 (1984) (Immune serum treatment of Junin virus infection), Lerman, et al., Rev. Argent. Microbiol., 18(1):33-5 (1986) (Homologous hyperimmune serum (HIS) for treating Junin virus), and Jahrling, J. Med. Virol., 12(2):93-102 (1983) (Lassa-immune plasma of guinea pig, primate, and human origin)).

(4) Small molecule anti-viral agents

Any small molecule anti-viral agents, when used alone or in combination with other compounds, that can alleviate, reduce, ameliorate, prevent, or place or maintain in a state of remission of clinical symptoms or diagnostic markers associated with viral hemorrhagic diseases or disorders, particularly those viral hemorrhagic diseases or disorders caused by infection of a Bunyaviridae, a Filoviridae, a Flaviviridae, or an Arenaviridae virus, can be used in the present combinations and methods.

For example, glycyrrhizinic acid and its derivatives for inhibition of Marburg virus reproduction (Pokrovskii, et al., Dokl Akad Nauk., 344(5):709-11 (1995)), Ribavirin (e.g., Ribavirin 2′, 3′, 5′-triacetate) for Inhibition of Dengue virus (Koff, et al., Antimicrob. Agents Chemother., 24(1):134-6 (1983)), Riba-virin for inhibition of Lassa virus (Jahrling, et al., J. Infect. Dis., 141 (5):580-9 (1980)), and Desferal (e.g., desferrioxamine), Ribavirin for inhibition of Marburg virus (Ignatyev et al., Voprosy Virusologil, 41:206-209 (1996) can be used.

2. Formulation and routes of administration

The compounds, blood-derived compositions and agents are preferably formulated as pharmaceutical compositions, preferably for single dosage administration. The concentrations of the compounds in the formulations or the protein concentration of the blood-derived composition are selected to be effective for delivery of an amount, upon administration, that is effective for the intended treatment. Typically, the compositions are formulated for single dosage administration.

To formulate a composition, the weight fraction of a compound or mixture thereof is dissolved, suspended, dispersed or otherwise mixed in a selected vehicle at an effective concentration such that the treated condition is relieved or ameliorated. Pharmaceutical carriers or vehicles suitable for administration of the compounds provided herein include any such carriers known to those skilled in the art to be suitable for the particular mode of administration.

Effective concentration of the blood-derived compositions can be empirically determined. Plasma and serum may be administered without further processing or processed according to known methods.

In addition, the compounds may be formulated as the sole pharmaceutically active ingredient in the composition or may be combined with other active ingredients. Liposomal suspensions, including tissue-targeted liposomes, may also be suitable as pharmaceutically acceptable carriers. These may be prepared according to methods known to those skilled in the art. For example, liposome formulations may be prepared as described in U.S. Pat. No. 4,522,811.

The active compound is included in the pharmaceutically acceptable carrier in an amount sufficient to exert a therapeutically useful effect in the absence of undesirable side effects on the patient treated. The therapeutically effective concentration may be determined empirically by testing the compounds in known in vitro and in vivo systems, such as the assays provided herein.

The concentration of active compound in the drug composition will depend on absorption, inactivation and excretion rates of the active compound, the physicochemical characteristics of the compound, the dosage schedule, and amount administered as well as other factors known to those of skill in the art.

Typically a therapeutically effective dosage The amounts administered may be on the order of 0.001 to 1 mg/ml, preferably about 0.005-0.05 mg/ml, more preferably about 0.01 mg/ml, of blood volume Pharmaceutical dosage unit forms are prepared to provide from about 1 mg to about 1000 mg and preferably from about 10 to about 500 mg, more preferably about 25-75 mg of the essential active ingredient or a combination of essential ingredients per dosage unit form. The precise dosage can be empirically determined.

The active ingredient may be administered at once, or may be divided into a number of smaller doses to be administered at intervals of time. It is understood that the precise dosage and duration of treatment is a function of the disease being treated and may be determined empirically using known testing protocols or by extrapolation from in vivo or in vitro test data. It is to be noted that concentrations and dosage values may also vary with the severity of the condition to be alleviated. It is to be further understood that for any particular subject, specific dosage regimens should be adjusted over time according to the individual need and the professional judgment of the person administering or supervising the administration of the compositions, and that the concentration ranges set forth herein are exemplary only and are not intended to limit the scope or use of the claimed compositions and combinations containing them.

Preferred pharmaceutically acceptable derivatives include acids, salts, esters, hydrates, solvates and prodrug forms. The derivative is typically selected such that its pharmacokinetic properties are superior to the corresponding neutral compound.

Thus, effective concentrations or amounts of one or more of the compounds provided herein or pharmaceutically acceptable derivatives thereof are mixed with a suitable pharmaceutical carrier or vehicle for systemic, topical or local administration to form pharmaceutical compositions. Compounds are included in an amount effective for ameliorating or treating the disorder for which treatment is contemplated. The concentration of active compound in the composition will depend on absorption, inactivation, excretion rates of the active compound, the dosage schedule, amount administered, particular formulation as well as other factors known to those of skill in the art.

Solutions or suspensions used for parenteral, intradermal, subcutaneous, or topical application can include any of the following components: a sterile diluent, such as water for injection, saline solution, fixed oil, polyethylene glycol, glycerine, propylene glycol or other synthetic solvent; antimicrobial agents, such as benzyl alcohol and methyl parabens; antioxidants, such as ascorbic acid and sodium bisulfite; chelating agents, such as ethylenediaminetetraacetic acid (EDTA); buffers, such as acetates, citrates and phosphates; and agents for the adjustment of tonicity such as sodium chloride or dextrose. Parenteral preparations can be enclosed in ampules, disposable syringes or single or multiple dose vials made of glass, plastic or other suitable material.

In instances in which the compounds exhibit insufficient solubility, methods for solubilizing compounds may be used. Such methods are known to those of skill in this art, and include, but are not limited to, using cosolvents, such as dimethylsulfoxide (DMS0), using surfactants, such as Tween®, or dissolution in aqueous sodium bicarbonate. Derivatives of the compounds, such as prodrugs of the compounds may also be used in formulating effective pharmaceutical compositions. For ophthalmic indications, the compositions are formulated in an ophthalmically acceptable carrier. For the ophthalmic uses herein, local administration, either by topical administration or by injection is preferred. Time release formulations are also desirable. Typically, the compositions are formulated for single dosage administration, so that a single dose administers an effective amount.

Upon mixing or addition of the compound with the vehicle, the resulting mixture may be a solution, suspension, emulsion or other composition. The form of the resulting mixture depends upon a number of factors, including the intended mode of administration and the solubility of the compound in the selected carrier or vehicle. If necessary, pharmaceutically acceptable salts or other derivatives of the compounds may be prepared.

The compound is included in the pharmaceutically acceptable carrier in an amount sufficient to exert a therapeutically useful effect in the absence of undesirable side effects on the patient treated. It is understood that number and degree of side effects depends upon the condition for which the compounds are administered. For example, certain toxic and undesirable side effects are tolerated when treating life-threatening illnesses that would not be tolerated when treating disorders of lesser consequence. The concentration of compound in the composition will depend on absorption, inactivation and excretion rates thereof, the dosage schedule, and amount administered as well as other factors known to those of skill in the art.

The compounds can also be mixed with other active materials, that do not impair the desired action, or with materials that supplement the desired action, such as cardiovascular drugs, antibiotics, anticoagulants and other such agents known to those of skill in the art for treating hemorrhagic viral infections, shock, infection, trauma and other disorders for which the treatments provided herein are contemplated.

Upon mixing or addition of the compound(s), the resulting mixture may be a solution, suspension, emulsion or the like. The form of the resulting mixture depends upon a number of factors, including the intended mode of administration and the solubility of the compound in the selected carrier or vehicle. The effective concentration is sufficient for ameliorating the symptoms of the disease, disorder or condition treated and may be empirically determined.

The formulations of the compounds and agents for use herein include those suitable for oral, rectal, topical, inhalational, buccal (e.g., sublingual), parenteral (e.g., subcutaneous, intramuscular, intradermal, or intravenous), transdermal administration or any route. The most suitable route in any given case will depend on the nature and severity of the condition being treated and on the nature of the particular active compound which is being used.

The formulations are provided for administration to humans and animals in unit dosage forms, such as tablets, capsules, pills, powders, granules, sterile parenteral solutions or suspensions, and oral solutions or suspensions, and oil-water emulsions containing suitable quantities of the compounds or pharmaceutically acceptable derivatives thereof. The pharmaceutically therapeutically active compounds and derivatives thereof are typically formulated and administered in unit-dosage forms or multiple-dosage forms. Unit-dose forms as used herein refers to physically discrete units suitable for human and animal subjects and packaged individually as is known in the art. Each unit-dose contains a predetermined quantity of the therapeutically active compound sufficient to produce the desired therapeutic effect, in association with the required pharmaceutical carrier, vehicle or diluent. Examples of unit-dose forms include ampules and syringes and individually packaged tablets or capsules. Unit-dose forms may be administered in fractions or multiples thereof. A multiple-dose form is a plurality of identical unit-dosage forms packaged in a single container to be administered in segregated unit-dose form. Examples of multiple-dose forms include vials, bottles of tablets or capsules or bottles of pints or gallons. Hence, multiple dose form is a multiple of unit-doses which are not segregated in packaging.

The composition can contain along with the active ingredient: a diluent such as lactose, sucrose, dicalcium phosphate, or carboxymethylcellulose; a lubricant, such as magnesium stearate, calcium stearate and talc; and a binder such as starch, natural gums, such as gum acaciagelatin, glucose, molasses, polvinylpyrrolidine, celluloses and derivatives thereof, povidone, crospovidones and other such binders known to those of skill in the art. Liquid pharmaceutically administrable compositions can, for example, be prepared by dissolving, dispersing, or otherwise mixing an active compound as defined above and optional pharmaceutical adjuvants in a carrier, such as, for example, water, saline, aqueous dextrose, glycerol, glycols, ethanol, and the like, to thereby form a solution or suspension. If desired, the pharmaceutical composition to be administered may also contain minor amounts of nontoxic auxiliary substances such as wetting agents, emulsifying agents, or solubilizing agents, pH buffering agents and the like, for example, acetate, sodium citrate, cyclodextrine derivatives, sorbitan monolaurate, triethanolamine sodium acetate, triethanolamine oleate, and other such agents. Actual methods of preparing such dosage forms are known, or will be apparent, to those skilled in this art; for example, see Remington's Pharmaceutical Sciences, Mack Publishing Company, Easton, Pa., 15th Edition, 1975. The composition or formulation to be administered will, in any event, contain a quantity of the active compound in an amount sufficient to alleviate the symptoms of the treated subject.

Dosage forms or compositions containing active ingredient in the range of 0.005% to 100% with the balance made up from non-toxic carrier may be prepared. For oral administration, a pharmaceutically acceptable non-toxic composition is formed by the incorporation of any of the normally employed excipients, such as, for example pharmaceutical grades of mannitol, lactose, starch, magnesium stearate, talcum, cellulose derivatives, sodium crosscarmellose, glucose, sucrose, magnesium carbonate or sodium saccharin. Such compositions include solutions, suspensions, tablets, capsules, powders and sustained release formulations, such as, but not limited to, implants and microencapsulated delivery systems, and biodegradable, biocompatible polymers, such as collagen, ethylene vinyl acetate, polyanhydrides, polyglycolic acid, polyorthoesters, polylactic acid and others. Methods for preparation of these formulations are known to those skilled in the art.

For oral administration, the pharmaceutical compositions may take the form of, for example, tablets or capsules prepared by conventional means with pharmaceutically acceptable excipients such as binding agents (e.g., pregelatinized maize starch, polyvinyl pyrrolidone or hydroxypropyl methylcellulose); fillers (e.g., lactose, microcrystalline cellulose or calcium hydrogen phosphate); lubricants (e.g., magnesium stearate, talc or silica); disintegrants (e.g., potato starch or sodium starch glycolate); or wetting agents (e.g., sodium lauryl sulphate). The tablets may be coated by methods well-known in the art. The pharmaceutical preparation may also be in liquid form, for example, solutions, syrups or suspensions, or may be presented as a drug product for reconstitution with water or other suitable vehicle before use. Such liquid preparations may be prepared by conventional means with pharmaceutically acceptable additives such as suspending agents (e.g., sorbitol syrup, cellulose derivatives or hydrogenated edible fats); emulsifying agents (e.g., lecithin or acacia); non-aqueous vehicles (e.g., almond oil, oily esters, or fractionated vegetable oils); and preservatives (e.g., methyl or propyl-p-hydroxybenzoates or sorbic acid).

Formulations suitable for rectal administration are preferably presented as unit dose suppositories. These may be prepared by admixing the active compound with one or more conventional solid carriers, for example, cocoa butter, and then shaping the resulting mixture.

Formulations suitable for topical application to the skin or to the eye preferably take the form of an ointment, cream, lotion, paste, gel, spray, aerosol, or oil. Carriers which may be used include vaseline, lanoline, polyethylene glycols, alcohols, and combinations of two or more thereof. The topical formulations may further advantageously contain 0.05 to 15 percent by weight of thickeners selected from among hydroxypropyl methyl cellulose, methyl cellulose, polyvinylpyrrolidone, polyvinyl alcohol, poly (alkylene glycols), poly/hydroxyalkyl, (meth)acrylates or poly(meth)acrylamides. The topical formulations is most often applied by instillation or as an ointment into the conjunctival sac. It, however, can also be used for irrigation or lubrication of the eye, facial sinuses, and external auditory meatus. It may also be injected into the anterior eye chamber and other places. The topical formulations in the liquid state may be also present in a hydrophilic three-dimensional polymer matrix in the form of a strip, contact lens, and the like from which the active components are released.

For administration by inhalation, the compounds for use herein can be delivered in the form of an aerosol spray presentation from pressurized packs or a nebulizer, with the use of a suitable propellant, e.g., dichlorodifluoromethane, trichlorofluoromethane, dichlorotetrafluoroethane, carbon dioxide or other suitable gas. In the case of a pressurized aerosol, the dosage unit may be determined by providing a valve to deliver a metered amount. Capsules and cartridges of, e.g., gelatin, for use in an inhaler or insufflator may be formulated containing a powder mix of the compound and a suitable powder base such as lactose or starch.

Formulations suitable for buccal (sublingual) administration include lozenges containing the active compound in a flavored base, usually sucrose and acacia or tragacanth; and pastilles containing the compound in an inert base such as gelatin and glycerin or sucrose and acacia.

The compounds may be formulated for parenteral administration by injection, e.g., by bolus injection or continuous infusion. Formulations for injection may be presented in unit dosage form, e.g., in ampules or in multi-dose containers, with an added preservative. The compositions may take such forms as suspensions, solutions or emulsions in oily or aqueous vehicles, and may contain formulatory agents such as suspending, stabilizing and/or dispersing agents. Alternatively, the active ingredient may be in powder form for constitution with a suitable vehicle, e.g., sterile pyrogen-free water or other solvents, before use.

Formulations suitable for transdermal administration may be presented as discrete patches adapted to remain in intimate contact with the epidermis of the recipient for a prolonged period of time. Such patches suitably contain the active compound as an optionally buffered aqueous solution of, for example, 0.1 to 0.2 M concentration with respect to the said active compound. Formulations suitable for transdermal administration may also be delivered by iontophoresis (see, e.g., Pharmaceutical Research 3 (6), 318 (1986)) and typically take the form of an optionally buffered aqueous solution of the active compound.

In addition to the common dosage forms set out above, the pharmaceutical compositions may also be administered by controlled release means and/or delivery devices such as those described in U.S. Pat. Nos. 3,536,809; 3,598,123; 3,630,200; 3,845,770; 3,847,770; 3,916,899; 4,008,719; 4,687,610; 4,769,027; 5,059,595; 5,073,543; 5,120,548; 5,354,566; 5,591,767; 5,639,476; 5,674,533 and 5,733,566.

Also provided are combinations for carrying out the therapeutic regimens. Such combinations, which may be packaged in the form of kits, contain one or more containers with therapeutically effective amounts of one or more tetracycline compounds and an anti-viral-hemorrhagic agent, in pharmaceutically acceptable form. The tetracycline compounds and the anti-viral-hemorrhagic agent, either separately or in a mixture, may be in the form of a pharmaceutically acceptable solution, e.g., in combination with sterile saline, dextrose solution, or buffered solution, or other pharmaceutically acceptable sterile fluid. Alternatively, the tetracycline compound and the anti-viral-hemorrhagic agent, either separately or in a mixture, may be lyophilized or desiccated; in this instance, the kit optionally further comprises in a container a pharmaceutically acceptable solution (e.g., saline, dextrose solution, etc.), preferably sterile, to reconstitute the tetracycline compound and the anti-viral-hemorrhagic agent to form a solution for injection purposes.

In another embodiment, a kit further comprises a needle or syringe, preferably packaged in sterile form, for injecting the complex, and/or a packaged alcohol pad. Instructions are optionally included for administration of the tetracycline compound and the anti-viral-hemorrhagic agent by a clinician or by the patient.

The magnitude of a therapeutic dose of the tetracycline compound(s), alone or in combination with the anti-viral-hemorrhagic agent will vary with the severity of the condition to be treated and the route of administration. The dose, and perhaps dose frequency, will also vary according to the age, body weight, condition and response of the individual patient. Dosage and administration may be empirically determined.

Desirable blood levels may be maintained by a continuous infusion of the tetracycline compound(s) and/or the anti-viral-hemorrhagic agent as ascertained by plasma levels. It should be noted that the attending physician would know how to and when to terminate, interrupt or adjust therapy to lower dosage due to toxicity, or bone marrow, liver or kidney dysfunctions. Conversely, the attending physician would also know how to and when to adjust treatment to higher levels if the clinical response is not adequate (precluding toxic side effects).

The efficacy and/or toxicity of the tetracycline compound(s), alone or in combination with the anti-viral-hemorrhagic agent can also be assessed by the methods known in the art, i.e., in animal models and/or clinical studies. For example, the efficacy and/or toxicity can be assessed in the animal models described in the following literatures: Huggins et al., J. Infect. Dis., 179(Supp1):S240-247 (1999) (ebola virus leathal mouse model); Lupton et al., Lancet, 2(8207):1294-5 (1980) (ebola virus guineapig model); Johnson et al., J. Virol., 73(1):783-786 (1999) (Dengue virus mouse model); Campetella et al., J. Med. Virol., 26(4):443-51 (1988) (Junin virus murine model); de Guerreol et al., J. Med. Virol., 15(2):197-202 (1985) (Junin virus guineapig model); Boxaca et al., Acta Virol., 28(3):198-203 (1984) (Junin virus guineapig model); Blejer et al., Medicina (B Aires), 43(6Pt2):898 (1983) (Junin virus rat model); and Frigerio et al., Medicina (B Aires), 38(5):603-4 (1978) (experimental model in Argentinean hemorrhagic fever).

Any suitable route of administration may be employed for providing the patient with an effective dosage of the tetracycline compound(s), alone or in combination with the anti-viral-hemorrhagic agent. For example, oral, transdermal, iontophoretic, parenteral (subcutaneous, intramuscular, intrathecal and the like) may be employed. Dosage forms include tablets, troches, cachet, dispersions, suspensions, solutions, capsules, patches, and the like. (See, Remington's Pharmaceutical Sciences).

The active compounds or pharmaceutically acceptable derivatives may be prepared with carriers that protect the compound against rapid elimination from the body, such as time release formulations or coatings.

Finally, the compounds may be packaged as articles of manufacture containing packaging material, a compound or suitable derivative thereof provided herein, which is effective for treatment of a viral hemorrhagic disease, within the packaging material, and a label that indicates that the compound or a suitable derivative thereof is for treating hemorrhagic diseases or shock or other disorder contemplated herein. The label can optionally include the disorders for which the therapy is warranted.

E. Blood-derived Compositions and Methods of Treatment

1. Blood-derived compositions and processes for producing compositions for treating diseases and disorders characterized by or associated with acute inflammatory responses

Also provided herein, are methods for preparing blood-derived compositions for treatment of the diseases and disorders characterized by or associated with acute inflammatory responses. The diseases and disorders contemplated herein include, but are not limited to, the viral hemorrhagic fevers, bacterial sepsis, viral hemorrhagic diseases as well as any disorder involving a cytotoxic immune response, including, but not limited to sepsis, cachexia, rheumatoid arthritis, chronic myelogenous leukemia and transplanted bone marrow-induced graft-versus-host disease, septic shock, immune complex-induced colitis, cerebrospinal fluid inflammation, autoimmune disorders, multiple sclerosis and other such disorders that involve release of inflammatory response mediators, including tumor necrosis factor (TNF) interleukins, particularly IL-1, and other interleukins including IL-6 and IL-8, chemokines platelet-activating factor (PAF), prostaglandins and leukotrienes (see, e.g., (1991) Ann. Intern. Med. 115: 464-466 for a more comprehensive listing).

Processes for producing these compositions are provided. The compositions are produced by contacting blood or fraction thereof either in vitro or in vivo with one or more tetracycline or tetracycline-like compounds in an sufficient amount and for a sufficient time to produce a response that is assessed by measuring the level of IL-1 and/or TNF receptors, using any standard assay, and looking for about a 3-fold or greater increase. The resulting blood or composition can be processed further or injected, preferably into a species and blood-type matched mammalian recipient.

Further processing can be used to isolate fractions thereof that exhibit the anti-inflammatory properties of the unfractionated properties. Fractions include, but are not limited to, the γ-globuline fraction, the AHF (anti-hemophilia factor, the albumin fraction, serum and plasma. Each fraction can be tested in model systems, such as those exemplified herein (see EXAMPLES) to identify active fractions. In addition or alternatively, fractions of interest are those that contain TNF and/or IL-1 receptors. The TNF and IL-1 receptors serve as indicators of the fractions of interest which contain other components that may contribute to the observed effectiveness of the blood-derived fractions in treating the acute inflammatory disorders.

In one embodiment, the process includes the steps of administering one or more tetracycline or tetracycline-like compound(s) to a mammal; b) collecting blood from the mammal; and c) recovering serum or plasma from the collected blood. Before step a) the baseline level of an indicator of stimulation is obtained. Preferably the level of IL-1 or TNF receptors is assessed, although the level of other cytokines and receptors, such as IL-16 (LCF—chemotactic for CD4, T-lymphocytes), or IL-2 receptors, is assessed using standard methods (i.e., R&D Systems, makes a variety of reagents to test for interleukins and receptors therefor). In some instances and for certain diseases, cells that produce particular factors may be identified, and those cells stimulated in vitro or in vivo to produce compositions for treatment of those diseases.

The resulting recovered serum and plasma can be used to administer to mammals exhibiting an acute inflammatory response, such as that associated with infection with a hemorrhagic virus or otherwise exhibiting symptoms of a septic reaction, such as shock, and the other disorders enumerated herein or known to involve a deleterious inflammatory response. The plasma or serum can be further fractionated and tested in model systems to identify active fractions. Any tetracycline or tetracycline-like compound provided herein or known to those of skill in the art is contemplated for use.

For in vitro preparation, blood or a fraction thereof is contacted with a tetracycline or tetracycline-like compound(s) or other agent, such as a virus, for time sufficient to observe at least a three-fold increase from baseline in the level of TNF or IL-1 receptors. The medium from the blood or fraction is isolated and further processed, such as by further fractionation, or concentration, and then it is administered to a mammal with an acute inflammatory disease, condition or disorder.

In one embodiment, white cells are harvested from the buffy coat of blood. The cells are treated, for example with Sendei virus to stimulate production of α-interferon, and the supernatant or medium from the cells is isolated. Any process whereby TNF, or IL-1 receptors can be generated, in vitro or in vivo can be used, and the resulting blood product or a derivative thereof administered.

a. Preparation of Serum and Plasma

Serum or plasma can be recovered from the collected blood by any methods known in the art. In one specific embodiment, the serum or plasma is recovered from the collected blood by centrifugation. Preferably, the centrifugation is conducted in the presence of a sealant having a specific gravity greater than that of the serum or plasma and less than that of the blood corpuscles which will form the lower, whereby upon centrifugation, the sealant forms a separator between the upper serum or plasma layer and the lower blood corpuscle layer. The sealants that can be used in the processes include, but are limited to, styrene resin powders (Japanese Patent Publication No. 38841/1973), pellets or plates of a hydrogel of a crosslinked polymer of 2-hydroxyethyl methacrylate or acrylamide (U.S. Pat. No. 3,647,070), beads of polystyrene bearing an antithrombus agent or a wetting agent on the surfaces (U.S. Pat. No. 3,464,890) and a silicone fluid (U.S. Pat. Nos. 3,852,194 and 3,780,935). In a preferred embodiment, the sealant is a polymer of unsubstituted alkyl acrylates and/or unsubstituted alkyl methacrylates, the alkyl moiety having not more than 18 carbon atoms, the polymer material having a specific gravity of about 1.03 to 1.08 and a viscosity of about 5,000 to 1,000,000 cps at a shearing speed of about 1 second⁻¹ when measured at about 25° C. (U.S. Pat. No. 4,140,631).

In another specific embodiment, the serum or plasma is recovered from the collected blood by filtration. Preferably, the blood is filtered through a layer of glass fibers with an average diameter of about 0.2 to 5μ and a density of about 0.1 to 0.5 g/cm³, the total volume of the plasma or serum to be separated being at most about 50% of the absorption volume of the glass fiber layer; and collecting the run-through from the glass fiber layer which is plasma or serum (U.S. Pat. No. 4,477,575). Also preferably, the blood is filtered through a layer of glass fibers having an average diameter 0.5 to 2.5μ impregnated with a polyacrylic ester derivative and polyethylene glycol (U.S. Pat. No. 5,364,533). More preferably, the polyacrylic ester derivative is poly(butyl acrylate), poly(methyl acrylate) or poly(ethyl acrylate), and (a) poly(butyl acrylate), (b) poly(methyl acrylate) or poly(ethyl acrylate) and (c) polyethylene glycol are used in admixture at a ratio of (10-12):(1-4):(1-4). In still another specific embodiment, the serum or plasma is recovered from the collected blood by treating the blood with a coagulant containing a lignan skelton having oxygen-containing side chains or rings (U.S. Pat. No. 4,803,153). Preferably, the coagulant comprises a lignan skelton having oxygen-containing side chains or rings, e.g., d-sesamin, I-sesamin, paulownin, d-asarinin, I-asarinin, 2α-paulownin, 6α-paulownin, pinoresinol, d-eudesmin, I-pinoresinol β-D-glucoside, I-pinoresinol, I-pinoresinol monomethyl ether β-D-glucoside, epimagnolin, lirioresinol-B, syringaresinol (dl), lirioresinonB-dimethyl ether, phillyrin, magnolin, lirioresinol-A, 2α, 6α-d-sesamin, d-diaeudesmin, lirioresinol-C dimethyl ether (ddiayangambin) and sesamolin. More preferably, the coagulant is used in an amount ranging from about 0.01 to 50 g per 1 L of the blood.

b. Further Fractionation of Plasma

Blood plasma or sera can be further separated into different fractions, including, inter alia, an albumin-containing fraction, a globulin-containing fraction and an AHF-containing fraction. Methods for preparing these fractions are known in the arts. Generally, these methods comprise one or more of the following procedures: (a) fractional precipitation with ammonium sulfate and similar salts; (b) organic solvent precipitation with cold ethanol or acetone and other such alcohols and ketones; (c) selective adsorption on calcium phosphate gels or with barium sulfate; (d) isoelectric precipitation by pH adjustment to the point at which there is no net charge on a given protein; and (e) chromatography by use of adsorbents such as CM- or DEAE-cellulose or by “Sephadex” gel filtration. Other procedures for selectively fractionating and purifying blood proteins involve the use of amino acids such as glycine and beta alanine, water-soluble organic polymers such as polyethylene glycol and polypropylene glycol, and water-insoluble polyelectrolyte polymers containing basic amino groups such as the dimethylaminopropylimide group.

(1) Preparation of Albumin-Containing Fraction

The plasma can further be separated into a fraction containing albumin by any methods known in the art. In one specific embodiment, the albumin-containing fraction is prepared by selective precipitation with block copolymers of ethylene oxide and polyoxypropylene polymer from the plasma (U.S. Pat. No. 4,025,500).

In another specific embodiment, the albumin-containing fraction is prepared by: (a) diluting the plasma in liquid form with a NaCI solution containing disodium ethylene dinitrilo tetraacetate and an albumin stabilizer; (b) adjusting the pH of the plasma solution resulting from step (a) to about 6.2; (c) heating the plasma solution from step (b) at about 60° C. for about 1½ hours; (d) cooling the plasma solution to about 10° C.; (e) precipitating impurities from the solution with polyethylene glycol at a concentration of about 18-20% with the albumin remaining in the supernatant; (f) isoelectrically precipitating albumin from the supernatant at a pH of about 4.6; and (g) recovering the albumin-containing fraction (U.S. Pat. No. 4,164,496). Preferably, the albumin stabilizer is sodium caprylate.

In still another specific embodiment, the albumin-containing fraction is prepared by: (a) adjusting the pH of the plasma in liquid form to about 6.7; (b) heating the plasma at about 60° C. for about 1½ hours; (c) adjusting the pH of the plasma to about 5.7; (d) precipitating impurities from the plasma by the addition of ethanol in an amount sufficient to give a final concentration of about 40 to 44% in the plasma along with cooling of the plasma to about −5° C., with the albumin remaining in the supernatant; and (e) precipitating albumin-containing fraction from the supernatant at a pH of about 4.8. (U.S. Pat. No. 4,222,934).

A blood group substance can be removed from the albumin-containing fraction. It can be removed for example, by treating the albumin-containing fraction with polyethylene glycol at pH of about 6.6 to 8.0, the effective polyethylene glycol concentration in the aqueous albumin solution being about 13 to 20% (w/v), in the presence of an inorganic salt at a concentration of at most 50 g/liter measured as sodium chloride and at a temperature in the range of about 2° C. to 30° C., the resulting polyethylene glycol/albumin solution having a protein concentration of about 5 to 40 g/liter, thereby precipitating and removing contaminant proteins containing the blood-group substance (U.S. Pat. No. 4,197,238).

Alternatively, the a blood group substance can be removed from the albumin-containing fraction by treating the albumin-containing fraction with polyethylene glycol at pH of about 8.0 to 9.6, the effective polyethylene glycol concentration in the aqueous albumin solution being about 15 to 30% (w/v), in the presence of an inorganic salt at a concentration of at most 50 g/liter measured as sodium chloride and at a temperature in the range of about 2° C. to 30° C., the resulting polyethylene glycol/albumin solution having a protein concentration of about 5 to 40 g/liter, thereby precipitating and removing contaminant proteins containing the blood-group substance (U.S. Pat. No. 4,197,238).

In another alternative method, the steps for removing a blood group substance from the albumin-containing fraction include treating the albumin-containing fraction with polyethylene glycol having an average molecular weight in the range of about 2,000 to 10,000 at pH of about 6.6 to 9.6, the effective polyethylene glycol concentration in the aqueous albumin solution being about 13 to 20% (w/v), in the presence of an inorganic salt at a concentration of at most 50 g/liter measured as sodium chloride and at a temperature in the range of about 2° C. to 30° C., the resulting polyethylene glycol/albumin solution having a protein concentration of about 5 to 40 g/liter, thereby precipitating and removing contaminant proteins containing the blood-group substance (U.S. Pat. No. 4,197,238).

Polymer content and α1-AGP content can be reduced in the albumin-containing fractiom such as by subjecting the albumin-containing fraction to ion exchange separation using an anion exchanger, the anion exchange separation is carried out at a pH ranging from about 5.1 to 5.5 (U.S. Pat. No. 5,277,818).

(2) Preparation of Globulin-Containing Fraction

The globulin-containing fraction can be prepared according to any methods known in the art. For example, conventional methods such as Cohn alcohol fractionating process (Kistler et al. (1962) Vox Sang, 7:414); and Cohn et al. (1946) J. Am. Chem. Soc. 68:459-475) and the Rivanol ammonium sulfate fractionation (Horejsi et al. (1956) Acta Med. Scand. 155: 65) can be used.

Alternatively, other known methods can be used (see, e.g., U.S. Pat. Nos. 4,347,138 and 5,310,877). U.S. Pat. No. 4,347,138 describes a method of separating serum albumin and a serum γ-globulin from each other in a solution using a semipermeable membrane by forcing the blood serum protein mixture solution through an ultrafiltration membrane having a cut off molecular weight of about 100,000 and composed of an aromatic polyether sulfone, while adjusting the total protein concentration and salt concentration in the mixture solution to not more than 4 g/dl and not more than 0.6 mole/I, respectively, and also adjusting the pH of the solution to a value of from about 3.8 to about 4.7. Preferably, the pH of the blood serum protein mixture solution is adjusted to a value of from 3.9 to 4.3. Also preferably, the salt contained in the blood serum protein mixture solution is sodium chloride or other physiologically acceptable salt.

U.S. Pat. No. 5,310,877 describes a method for the separation of gamma globulin from albumin contained in an aqueous solution of both by ultrafiltration using a microfilter having a water permeability of 0.2-25 gallons per square foot per day per pound per square inch including a porous solid filter substrate one surface of which is impregnated with particulate solids affixed within the pores of the substrate having an average particle size of about 0.1-0.5 micrometer at the feed interface, the aqueous solution being characterized in that the total concentration of protein in the aqueous solution is about 0.1-2% by weight, the pH of the aqueous solution is 8-10 and the solution contains no more than about 0.01 mole per liter of inorganic electrolyte, the albumin being enriched in the retentate and the gamma globulin being enriched in the permeate. Preferably, the particulate solids being used are titanium oxide particles. Also preferably, the substrate being used is sintered stainless steel.

Since intravenous administration is more direct and efficient, it is sometimes desirable or necessary to administer the globulin-containing fraction intravenously. A globulin-containing fraction prepared by the conventional fractionation contains anti-complement activity, i.e., the property of fixing complement non-specifically (U.S. Pat. No. 4,082,734). This anti-complement activity is related to the formation of aggregates. Such globulin-containing fraction containing the anti-complement activity is not suitable for intravenous administration because the fraction can cause shock in some patients (U.S. Pat. No. 4,124,576). Therefore, the anti-complement activity must be eliminated or reduced before the globulin-containing fraction can be administered intravenously.

The anti-complement activity can be eliminated or reduced according to any methods known in the art. For example, pepsin decomposition (Schultze and Schwick, Dtsch. Med. Wochenschrift, 87:1643 (1962)); decomposition (Barandun, et al., Vox Sang., 28:157 (1975)); HCl treatment (Barandun, et al., Vox Sang., 7:187 (1962)) and β-propiolactone treatment (Stephan, Z. Klin. Chem. Klin. Biochemie, 7:282 (1969)) can be used. In other specific embodiments, the processes described in U.S. Pat. Nos. 4,082,734, 4,075,193, 4,124,576, 4,154,819, 4,374,763, 4,436,724, 4,835,257

U.S. Pat. No. 4,082,734 describes a method of preparing an intravenously applicable globulin of substantially unchanged half-life but free from anti-complement activity, by heating plasma or serum for about 2 to 4 hours at about 50° C. to 56° C., and then fractionating, the heating having been long enough within the recited parameters so that the product upon fractionation is substantially free from anti-complement activity. Preferably, the fractionation is effected with alcohol or ammonium sulfate. Also preferably, the heating is effected for about 2 hours at about 56° C.

U.S. Pat. No. 4,075,193 describes a process for producing globulin for intravenous administration which comprises: 1) adsorbing plasminogen derived from blood of a selected mammalian species on an adsorbent substrate of L-lysine agarose; 2) washing the adsorbate to elute impurities; 3) eluting the purified plasminogen from the substrate; 4) converting the eluted plasminogen to plasmin; 5) incubating a mixture of the plasmin and a quantity of homospecific immune globulin having anticomplementary activity under conditions such that the anticomplementary activity is substantially reduced; and 6) inactivating plasmin present in the mixture by adsorption on an inactivation adsorbent for plasmin, and recovering the immune globulin.

U.S. Pat. No. 4,124,576 describes a process for preparing a gamma globulin substantially devoid of anticomplementary activity and suitable for intravenous administration, from a material selected from the Cohn Fraction II+III plasma protein paste having a protein content of about 25-30%, Cohn Fraction II paste and placental extracts containing these fractions which comprises the steps: 1) suspending the paste in water to form a solution of low ionic strength having a conductance of about 300×10⁻⁶ cm⁻¹ ohm⁻¹ at a pH of about 4.9 to 6.0 to produce a precipitate and a filtrate; 2) precipitating impurities from the filtrate by adding polyethylene glycol to 4% (w/v); 3) further precipitating impurities by the addition of ethanol in a concentration of from 4 to 12% (w/v); and 4) precipitating the gamma globulin by adding polyethylene glycol to 10 to 12% (w/v) or by adding ethanol to 20 to 30% (v/v), preferably 25% (v/v) at a pH of from 7 to 8.2, preferably 8.0, the process being carried out at a temperature of about 0-6° C.

U.S. Pat. No. 4,154,819 describes a process for preparing a γ-globulin solution suitable for the intravenous application by treating the solution of γ-globulin with acetimido ethyl ester hydrochloride, diketene, formimido ethyl ester hydrochloride or propanesultone at a pH of about 9, thereafter adjusting the pH to about 7 to 7.5, and separating the solution from the solids by dialysis or fractionation followed by sterile filtration. Preferably, the diketene is employed in about 0.02 g per g of protein in the γ-globulin solution. U.S. Pat. No. 4,374,763 describes a process for producing γ-globulin suitable for use in intravenous administration and of an anticomplementary activity of lower than 20% by bringing Cohn's Fraction II for the gamma-globulin into suspension in an aqueous solution of a monosaccharide, disaccharide or sugar alcohol, adjusting the pH of the suspension to about 7.0 to 9.0, adding dextran of an average molecular weight of 10,000 to 70,000 into the suspension to produce an aqueous about 2 to 10% (w/v) solution of dextran, and after removing the thus formed precipitate, adding ammonium sulfate to the mother liquor to precipitate the gamma-globulin.

U.S. Pat. No. 4,436,724 describes a method for producing γ-globulin which can be administered intravenously without adverse reactions. The method includes treating γ-globulin with pepsin or uropepsin in a neutral pH range of about 6.0 to 7.5. The aggregates in γ-globulin are selectively decomposed, while any decomposition of monomer γ-globulin molecule is substantially prevented. The globulin-containing fraction thus produced with reduced anti-complementary activity is stabilized by adding uropepsin which serves simultaneously as a proteolytic enzyme and a stabilizer.

U.S. Pat. No. 4,835,257 describes a process for the preparation of gamma globulin suitable for intravenous administration. The process includes the steps of: dissolving gamma globulin precipitated from blood or blood products in a solution, separating non-dissolved precipitate from the solution, adding polyethylene glycol to the separated solution, separating precipitate from the polyethylene glycol solution, increasing the polyethylene glycol concentration in the solution, separating precipitated purified gamma globulin from the higher concentrated polyethylene glycol solution, dissolving the purified gamma globulin in a solution suitable for intravenous administration. The process also includes a step of dissolving the gamma globulin precipitated from blood in a solution having a neutral pH, adding polyethylene glycol in the first step to a concentration of about 4.0-5.5% by weight, and increasing the polyethylene glycol concentration in the second step to at least 9% but not more than 16% by weight, and by adding a buffer to the solution just prior to adding the polyethylene glycol in one of the two polyethylene glycol addition steps.

In another specific embodiment, the globulin-containing fraction can be lyophilized for extended shelf-life and ease of transportation. The globulin-containing fraction can be lyophilized by any methods known in the art, preferably in the presence of salts or sugars. For example, the processes described in the U.S. Pat. Nos. 4,168,303 and 4,692,331 can be used.

U.S. Pat. No. 4,168,303 describes a process for producing a lyophilized gamma globulin preparation for intravenous administration, which comprises freeze-drying an aqueous solution of gamma globulin which has undergone no modification and has an anticomplementary activity of 20 (C′H50) or less in the presence of about 0.06 to 0.26 part by weight of sodium chloride for 1 part by weight of the gamma globulin. Preferably, the freeze drying is carried out in the presence of about 0.1 to 0.3 part by weight of serum albumin for 1 part by weight of the gamma globulin. Also preferably, the freeze drying is carried out in the presence of about 0 to 0.5 part by weight of a diluent for about 1 part by weight of the gamma globulin. Further preferably, the diluent is mannitol.

U.S. Pat. No. 4,692,331 describes a process for preparing a storage-stable, intravenously administrable γ-globulin dry preparation, which γ-globulin has been obtained by fractionating plasma with polyethylene glycol and has been substantially freed of remaining polyethylene glycol. The process includes the steps of: (1) adding glucose to an aqueous solution of γ-globulin, which is substantially free of remaining polyethylene glycol and is suitable for intravenous administration, the amount of glucose added being from about 0.2 to 2.0 parts by weight, based on one part of γ-globulin sufficient to stabilize the γ-globulin; and thereafter (2) lyophilizing the aqueous solution to produce a dry powder. Preferably, the aqueous solution contains γ-globulin in an amount of about 5 to 20% (W/V) in terms of protein.

(3) Preparation of AHF-Containing Fraction

Factor VIII and von Willebrand's factor are associated plasma proteins that together are called Antihemophilic Factor (AHF). Both are important in the blood clotting mechanism. Methods of making concentrates of AHF are known in the art. These range from simply freezing and then thawing plasma (cryoprecipitation) to yield a more concentrated insoluble mixture of Factor VIII, fibrinogen, cold-insoluble globulin to more involved procedures (e.g., Pool et al. New England Journal of Medicine, 273:1443-1447 (1965)). These concentrates may be made more highly purified by further treatment employing techniques such as aluminum hydroxide absorption, glycine extraction, polyethylene glycol concentration, and filtration. The AHF-containing fraction can be prepared according to the above described processes. Alternatively, the processes described in the U.S. Pat. Nos. 3,631,018, 3,652,530, 3,682,881, 3,973,002, 4,069,216 4,089,944, 4,104,266, 4,170,639, 4,203,891, 4,210,580, 4,251,437, 4,289,691, 4,348,315, 4,383,989, 4,386,068, 4,404,131, 4,435,318, 4,522,751, 4,543,210, 4,743,680, 4,814,435, 4,952,675, 4,977,246, 5,484,890, H1,509 and Re. 29,698 can be used.

U.S. Pat. No. 3,631,018 describes a method for preparing a concentrate of AHF including fractionating a cryoprecipitate concentrate of AHF with polyethylene glycol and glycine in a three-step precipitation: (1) first with about 3-4% by weight of polyethylene glycol followed by recovery of the supernate; (2) then with polyethylene glycol added to about 10% by weight followed by recovery of the resulting precipitate; and (3) finally with about 1.3-1.8 M glycine added to a solution of the precipitate from step (2) followed by recovery of the resulting precipitate. The polyethylene glycol suitable for use in the method has a molecular weight in the range of 200-20,000, preferably 400-6,000, more preferably about 4,000.

U.S. Pat. No. 3,652,530 describes a method of preparing highly purified AHF by treating an extract of a precipitate obtained by cryoethanol precipitation with polyethylene glycol in three successive precipitations, first with aluminum hydroxide gel at pH about 5.6-7.0, then with polyethylene glycol to a concentration of about 3.0-6.5%, and finally with added polyethylene glycol to a concentration of 10-12% to obtain a precipitate containing the highly purified AHF.

U.S. Pat. No. 3,682,881 describes a method for the preparation of a prothrombin complex and an AHF concentrate from citrated blood plasma treated with 1.5-1.8 M glycine. The resulting precipitate was treated successively with polyethylene glycol, first to a concentration of 3-4% and then 10% by weight, and finally with 1.8 M glycine.

U.S. Pat. No. 3,973,002 describes a method for isolating antihemophilic factor of human blood plasma including the steps of adjusting the pH of a solution of buffer-extracted plasma cryoprecipitate to from about 6.0 to about 7.0, and cooling the solution at a temperature of from about 2° C. to about 20° C. for from about 15 to about 60 minutes to cause precipitation of impurities.

U.S. Pat. No. 4,069,216 describes an improvement in the process described in U.S. Pat. No. 3,631,018 mentioned above, in which the process includes the step of holding a buffered solution of F. VIII and 6% polyol at 0-5° C. until precipitation occurs.

U.S. Pat. No. 4,089,944 describes a method for producing a clinically useful freeze-dried solid composition containing AHF and fibrinogen from blood plasma or an AHF-containing fraction thereof including the steps of fractionating the plasma to obtain a solid mixture containing AHF and fibrinogen, dissolving the solid mixture in an aqueous medium and freeze-drying the resulting solution to obtain a clinically useful freeze-dried solid composition which is then reconstituted in a reconstitution liquid for use, and including the step of rendering the freeze-dried, solid composition rapidly soluble in an aqueous medium at room temperature by adding water soluble carbohydrate to the mixture, the amount of carbohydrate added being an amount sufficient to produce at least about 2 grams per 100 milliliters concentration of carbohydrate upon reconstitution of the composition in a suitable medium to produce a therapeutically useful solution of AHF. Preferably, the carbohydrate used is dextrose, maltose, lactose or sucrose.

U.S. Pat. No. 4,104,266 describes a method for the preparation of purified AHF which includes the thawing of frozen plasma at a temperature of between about 0° C. and about 1° C. to obtain a cryoprecipitate containing AHF, and including the steps of: (a) extracting the cryoprecipitate with a low ionic strength buffer solution containing tris (hydroxymethyl) aminomethane at a temperature of about 0° C. to obtain a cold insoluble fraction having cold soluble impurities removed therefrom; (b) extracting the cold insoluble fraction with a low ionic strength buffer solution containing tris (hydroxymethyl) aminomethane at a temperature of about 21° C. to obtain a solution containing AHF and the buffer solution; (c) deprothrombinizing the solution with aluminum hydroxide gel; and (d) recovering an AHF-rich solution.

U.S. Pat. No. 4,170,639 describes a process for the production of antihemophilic factor concentrate in purified form having enhanced potency and solubility by: (a) subjecting an aqueous extract of antihemophilic blood plasma cryoprecipitate to purification by mixing with an aluminum hydroxide adsorbent at an acid pH and precipitating unwanted protein in the cold, the pH conditions being such that unwanted protein is selectively removed by adsorption without substantial loss of antihemophilic factor potency from the aqueous extract; (b) constituting the purified aqueous extract with buffer and saline and adjusting to an acid pH, and (c) freeze-drying the thus adjusted aqueous extract.

U.S. Pat. No. 4,203,891 describes a method of increasing the yield of antihemophilic factor VIII (AHF), from whole blood, blood plasma or blood plasma fractions by collecting the blood or plasma or plasma fraction from a donor directly into an anticoagulant agent selected from heparin, sodium heparin, or mixtures thereof, which agent does not reduce the physiological concentration of calcium, and recovering the AHF. Preferably, the anticoagulant is used in the range of 0.1-10 units/ml based on total volume of whole blood or blood plasma and the AHF is recovered by fractionation using glycine, ethanol, ethanolglycine, polyethylene glycol or glycine-polyethylene glycol precipitation.

U.S. Pat. No. 4,210,580 describes a process for separating and isolating AHF and fibronectin from plasma by cryoprecipitation (0-15° C.) in the presence of a sulfated mucopolysaccharide, e.g., heparin, to a concentration of about 0.15-0.25 mg/ml of plasma (approximately 22.5 to 37.5 units of heparin/mI of plasma). The resulting fibronectin precipitate is purified chromatographically and the heparin supernatant is mixed with an anion exchange resin such as DEAE cellulose with Heparasorb to remove heparin and to provide a supernatant having 90-95% of the original procoagulant activity.

U.S. Pat. No. 4,251,437 describes a process for producing an antihemophilic factor preparation (AHF) by thawing deep-frozen human blood plasma, at least partially, by irradiation with electromagnetic waves of a frequency of about 10⁸-10¹⁵ Hz for a period of time and with an energy penetration such that the temperature in the thawed blood plasma does not exceed 10° C. at any point, centrifuging the thawed product to form a cryoprecipitate, redissolving the cryoprecipitate in a buffer, isolating a concentrated solution, and optionally freeze-drying the concentrated solution. Preferably, the irradiation is controlled so that the temperature in the thawed product does not exceed 4° C. at any point. Also preferably, the irradiation is carried out with microwaves of a frequency of about 10⁸-3×10¹¹ Hz. Further preferably, the irradiation is carried out with microwaves of a frequency of about 2×10⁹-3×10¹⁰ Hz.

U.S. Pat. No. 4,289,691 describes a method for obtaining AHF from fresh blood plasma by adding heparin, used in the range of about 1-10 units/ml of plasma, to fresh plasma collected by plasmapheresis into a calcium chelating anticoagulant, freezing the plasma, resolubilizing the plasma, isolating a cryoprecipitate from the plasma, resolubilizing the cryoprecipitate, adding a citrate saline heparin buffer to the resolubilized cryoprecipitate, incubating the resolubilized, buffered cryoprecipitate at about 0-10° C. for a time in excess of about 1 hour in the presence of heparin precipitable cold insoluble globulin, separating an AHF rich precipitate and isolating AHF from the precipitate.

U.S. Pat. No. 4,348,315 describes a process for purifying and/or concentrating the F. VIII complex, starting from cryoprecipitate or Cohn Fraction I-O, by dissolving a composition containing F. VIII together impurities in 1.5 M glycine solution at 15° C. and pH 6.3-7.8 to obtain a solution containing F. VIII and a precipitate containing the impurities. Optionally, the process includes the additional step of adding PEG to the resulting F. VIII-containing glycine solution followed by precipitating and then concentrating purified F. VIII from the solution.

U.S. Pat. No. 4,383,989 describes a method of obtaining AHF by collecting freshly obtained plasma or plasma fractions directly into heparin, sodium heparin or mixtures thereof, in a proportion of about 6-8 units of heparin/mI of plasma, in the absence of a citrate buffer, and applying a cold incubation technique (0-10° C.) using heparin precipitable cold insoluble globulin.

U.S. Pat. No. 4,386,068 describes a process for producing an AHF concentrate by treating an aqueous suspension of cryoprecipitate containing AHF proteins with aluminum hydroxide gel, subjecting the resulting solution to ultrafiltration, and then constituting the solution resulting from the ultrafiltration in buffer and saline. Optionally, the solution resulting from the ultrafiltration may be treated with 1.6-2.2 M glycine for further purification.

U.S. Pat. No. 4,404,131 describes a method of producing an AHF concentrate by subjecting an AHF concentrate obtained by conventional fractionation, e.g., cryoprecipitation, to cryoalcohol precipitation.

U.S. Pat. No. 4,435,318 describes a process for the separation and recovery of Factor VIII, von Willebrand's factor, and Factor V from plasma and plasma derivative streams having a pH normally between about 6 to 8.5 by removing from the blood stream when present substantially all initial turbidity therein, subsequently passing the blood plasma into and out of an apparatus containing one or more semi-permeable membranes which separate the plasma stream from a salt receiving stream thereby decreasing the salt content of the plasma stream between about 45 to 80% to cause the formation of a protein turbidity enriched in Factor VIII, von Willebrand's factor and Factor V, subsequently removing substantially all of the turbidity and maintaining the temperature of the plasma stream during the separation and recovery process in the range of between about 4-40° C., and at substantially its original starting pH level.

U.S. Pat. No. 4,522,751 describes a method of producing a preparation containing Factor VIII (AHF) from a Factor-VIII-containing plasma fraction, the preparation containing Factor VIII (AHF) having a specific activity of at least 1.5 units of Factor VIII/mg protein, immunoglobulin G (IgG) of from 15 to 30 mg/1000 units of Factor VIII and fibrinogen of from 20 to 40 mg/100 units of Factor VIII, by: (a) dissolving the Factor-VIII-containing plasma fraction in a buffer solution containing a sulfated polysaccharide at a pH value approximately in the neutral range; (b) lowering the pH to a value ranging from 6.0 to 6.4 and adjusting the temperature to between about 0° C. to about 25° C. to precipitate undesired proteins and obtain a Factor-VIII-containing supernatant; (c) adding at least of glycine, sodium chloride and sodium citrate, to the Factor-VIII-containing supernatant to maintain the major part of the immunoglobulins contained in the supernatant in solution; (d) adding a protein precipitating agent to obtain a Factor-VIII-containing precipitate; and (e) dissolving the Factor-VIII-containing precipitate in a solvent to obtain the final product.

U.S. Pat. No. 4,543,210 describes a process for producing high purity antihemophilic factor concentrate from an antihemophilic factor-containing dispersion or solution isolated from blood plasma or a blood plasma fraction including performing two consecutive precipitations using a combination of precipitants in each precipitation, first a combination of 1-4% by weight, based on weight of solution, of polyethylene glycol and 0.1-0.2 ml of 1-3%, based on weight of suspension, aluminum hydroxide suspension per gram of protein in the starting dispersion or solution, followed by a combination of added polyethylene glycol to provide a final concentration of 9-13% by weight, based on weight of the resulting solution, and 10-20% by weight of glycine, based on weight of the polyethylene glycol solution, and 10-20% by weight, based on weight of the polyethylene glycol solution, of sodium chloride.

U.S. Pat. No. 4,743,680 describes a process for purifying a protein that has antihemophilic factor activity by column chromatography in a column behaving predominantly as an ion-exchange chromatography column, including the steps of: (a) equilibrating the chromatography column; (b) loading a sample containing the protein on the column, causing the protein to adsorb onto the column; (c) washing the column; (d) eluting the adsorbed protein from the column by causing it to desorb from the column; (e) recovering the protein in purified form; and also including the step of: adding to the column a substance containing of an effective amount for selectively increasing the electrostatic forces on the surface of the protein and concomitantly decreasing the hydrophobicity of the protein of a hydration additive selected from among sugars and polyhydric alcohols during at least one of the steps (a), (b), and (c) thereby promoting the adsorption of the protein on the column.

U.S. Pat. No. 4,814,435 describes a method for preparing a Factor VIII (AHF)-containing fraction having a specific activity of at least 2.5 units of Factor VIII/mg protein as well as a portion of immunoglobulin G (IgG) of 10 mg/1000 units of Factor VIII at most, with the risk of transmission of viral or bacterial infections avoided or largely reduced when applied therapeutically or prophylactically. The method includes the steps of: 1) preparing a first solution of a Factor VIII containing plasma fraction including at least one of a heparinoid and a complex compound of heparin and antithrombin III (Atheplex); 2) precipitating and separating undesired proteins from the first solution in the presence of sulfated polysaccharides at a pH of 6.0 to 6.4 and at a temperature of 0-25° C. so as to obtain a purified Factor VIII containing supernatant; 3) treating the purified Factor VIII containing supernatant with a protein precipitating agent selected from ammonium sulfate, ammonium sulfate-glycine, sodium chloride-glycine, sodium sulfate, sodium sulfa te-sodium citrate, ammonium sulfate-sodium citrate, sodium chloride-ammonium sulfate at a concentration of 8 to 35% and a pH of 5.6 to 6.8 so as to precipitate a Factor VIII containing precipitate; 4) dissolving the Factor VIII containing precipitate in a buffer solution so as to obtain a second solution; 5) one of ultrafiltering and dialyzing the second solution, and lyophilizing so as to obtain a lyophilizate; 6) and heat-treating the lyophilizate at a temperature and for a period of time sufficient to inactivate possibly present viruses.

U.S. Pat. No. 4,952,675 describes a process for purifying a protein having antihemophilic factor activity by column chromatography in a column behaving predominantly as a hydrophobic affinity chromatography column, including the steps of: (a) equilibrating the chromatography column; (b) loading a sample containing the protein on the column, causing the protein to adsorb onto the column; (c) washing the column; (d) eluting the adsorbed protein from the column by causing it to desorb from the column; (e) recovering the protein in purified form; and also including the step of: adding to the column a substance containing an effective amount for selectively increasing the electrostatic forces on the surface of the protein and concomitantly decreasing the hydrophobicity of the protein of a hydration additive selected from among sugars and polyhydric alcohols during the step (d) thereby promoting the desorption of the protein from the column; and subjecting the eluate containing the protein from the step (d) to a second purification using a second column behaving predominantly as an ion-exchange chromatography column prior to the step (e).

U.S. Pat. No. 4,977,246 describes a method for obtaining an AHF-rich product from human plasma by: (a) thawing freshly frozen human plasma at a temperature of about 6-10° C. to obtain a plasma solution; (b) adding one volume of about 1.20 M to 1.80 M aqueous solution of a precipitating agent selected from the group consisting of sodium citrate, potassium citrate and citric acid to two volumes of the plasma solution obtained in step (a) at a temperature of about 0-10° C. to form a precipitate; (c) incubating the precipitate-containing solution in an ice bath for about 20 to 40 minutes; and (d) separating the precipitate from the solution.

U.S. Pat. No. 5,484,890 describes a method of recovering, from a biological sample, an antihemophilic factor protein containing fraction having increased antihemophilic factor protein stabilit. The sample contains (a) an antihemophilic factor protein, (b) at least one destabilizing protease impurity, and (c) at least one proprotease impurity; and the fraction having at least 17 units of antihemophilic factor protein/mg of total protein; the method comprising: contacting the sample with an amount of a protease removing agent effective to remove a destabilizing amount of the protease impurity and an amount of proprotease removing agent effective to remove a destabilizing amount of the proprotease impurity. The proprotease removing agent includes an anion exchange resin in an amount ranging from 70 mg total loading protein/ml anion exchange resin to 750 mg total loading protein/ml anion exchange resin. U.S.

Patent No. H1,509 describes a process for producing a Factor VIII concentrate from blood plasma, by: (a) obtaining a cryoprecipitate containing Factor VIII from blood plasma; (b) dissolving the cryoprecipitate in an aqueous solution containing heparin in an amount sufficient to provide a cryoprecipitate/heparin solution containing from about 30 to about 150 units of heparin per milliliter of solution; (c) adding a sufficient amount of a precipitant consisting essentially of PEG to the cryoprecipitate/heparin solution while maintaining the solution at a temperature of from 20° C. to 30° C. to precipitate protein contaminants, leaving a PEG supernatant containing Factor VIII; (d) recovering the PEG supernatant; and (e) recovering Factor VIII from the PEG supernatant.

U.S. Pat. No. Re. 29,698 describes a method for improving the yield of AHF obtained from blood plasma and blood plasma fractions, obtained by cryoprecipitation, by the addition of heparin. The heparin-treated cryoprecipitate may then be further fractionated using polyethylene glycol and glycine. When the heparin-treated cryoprecipitate is further fractionated, heparin is preferably added twice, once to the initial cryoprecipitate and subsequently to the further fractionated concentrate.

(4) Preparation of Fraction Containing Soluble IL-1 Receptor or Soluble TNF Receptor

In one specific embodiment, the plasma is further separated into a fraction containing soluble IL-1 receptor or soluble TNF receptor. The preparation can be monitored by assaying for the physical properties of the receptors such as molecular weight, polarity, ionic strength, charge, isoelectric point, etc (Sambrook et al., Molecular Cloning: A Laboratory Manual (2nd Ed.), Cold Spring Harbor Laboratory Press, 1989). The preparation can also be monitored by assaying for the functional properties of the receptors such as the ability to specifically bind IL-1 or TNF, to block specific binding between IL-1 and IL-1 receptor or between TNF and a TNF receptor and to neutralize or reduce the biological activity of IL-1 or TNF. Preferably, the preparation is monitored by antibody-based assays and any anti-IL-1 soluble receptor and anti-TNF soluble receptor antibodies can be used (see Current Protocols in Immunology (Ed. Coligan et al.) John Wiley & Sons, Inc., 1997).

C. Methods of treatment using the resulting blood-derived compositions

The compositions thus produced are suitable for treating viral hemorrhagic diseases or disorders or other diseases, disorders or syndromes involving such cytotoxic responses including, but not limited to, other acute infectious diseases, sepsis, cachexia, rheumatoid arthritis and other autoimmune disorders, acute cardiovascular events, chronic myelogenous leukemia and transplanted bone marrow-induced graft-versus-host disease, septic shock, immune complex-induced colitis, cerebrospinal fluid inflammation, autoimmune disorders, multiple sclerosis. Accordingly, methods for treating or preventing a viral hemorrhagic disease or disorder or other such disorders involving such cytoxic responses in a mammal are provided. These methods include the steps of administering to the mammal an effective amount of the immune composition(s) produced according to the above processes.

Furthermore, such compositions can be used alone or in combination with a tetracycline or tetracycline-like compound(s) and/or other anti-viral-hemorrhagic agent(s), such as IL-1 inhibitors and TNF inhibitors. Any of the above noted disorders and disorders involving an acute inflammatory response can be treated by the compositions.

Viral hemorrhagic diseases can be treated by administration of tetracycline or tetracycline-like compound(s), The effectiveness of administration of a tetracycline compound or tetracycline-like compound(s) for treatment is optimal shortly after infection. Such treatment is preferably combined with administration of the compositions provided herein and/or other treatments for viral hemorrhagic disorders.

Methods for treating disorders involving acute inflammatory responses characterized by elevated and debilitating levels of cytokines are provided. These disorders include those enumerated herein and any others in which acute inflammatory responses, as assessed by elevated levels of TNF and/or IL-1, occur. Several methods are provided.

In one method a mammal determined to have an acute inflammatory response or a disease or condition characterized by such response is treated with a blood-derived composition provided herein. The mammal may also be treated with a tetracycline or tetracycline-like compound or plurality thereof and/or with a treatment known to have some effect on the symptoms of or on disorder. All treatments may be administered simultaneously, successively or intermittently and, as necessary, repeatedly and for a time sufficient to observe an amelioration or treatment of the symptoms of the disease, condition or disorder.

Hence, including among the methods provided herein, are methods in which such mammals are treated with blood or fraction thereof that has been contacted with a tetracycline or tetracycline-like compounds either in vitro or in vivo. Where the blood is treated in vivo, it is obtained from a donor who has been administered a tetracycline and tetracycline-like compounds prior to providing blood. Where the blood or a fraction thereof, particularly white blood cell-containing fraction, such as buffy coats, has been treated in vitro with a tetracycline and/or tetracycline-like compound(s), it is obtained from an untreated donor and then either fractionated prior to contacting or subsequent to contacting. In one embodiment, the blood is treated to obtain the buffy coat, which contains the white blood cells. The buffy coat fraction is contacted in vitro with a tetracycline and/or tetracycline-like compound(s). The medium from the treated cells is administered. It can be further fractionated or concentrated prior to administration. In all instances, the levels of the TNF and IL-1 receptors are monitored prior to contacting with the tetracycline and/or tetracycline-like compound(s), during and after contacting for at least a three-fold increase in the level of such receptors compared to the baseline, prior to contacting with the tetracycline and/or tetracycline-like compound(s). Such measure serves as indicator that the factors, which include sTNF receptors and/or IL-1 receptors, particularly IL-1 RA, have reached a sufficient level. These receptors serve as the marker for a sufficient level of induction of the palliative factors; they are not necessarily the only factors responsible for the observed effects.

These methods may also be combined with other methods for treating such disorders, such as other anti-IL-1 antibodies, anti-IL-1 receptor antibodies, IL-1 receptor antagonists, IL-1 production inhibitors, IL-1 receptor production inhibitors, and IL-1 releasing inhibitors.

Administration is effected by any suitable route, including systemic, local and topical administration, such as intramuscularly, intravenously, parenterally and orally. Typically, administration of a blood product will be via IV route. Administration of a tetracycline compound will be orally. Amounts of tetracycline is about 100-500 mg twice per day for one or more days, typically at least three and up to about ten days. These amounts are also the amounts for administration human donors to induce factors for preparation of the blood-derived compositions.

The disorders include hemorrhagic diseases and disorders, wasting diseases, sepsis, autoimmune disorders, particularly acute episodes associated with autoimmune disorders, acute episodes associated with multiple sclerosis, acute allergic reactions and other inflammatory diseases. The methods herein are particularly useful for treating hemorrhagic diseases or disorders, for which there have heretofore been few, if any, effective treatments.

In one method, a mammal suffering from such disorder is treated with an amount of a tetracycline and tetracycline-like compounds effective to ameliorate a symptom of the disorder, particularly, a disorder associated with elevated levels of cytokines associated with an acute inflammatory disorder. This method is intended for treatment of viral hemorrhagic fevers, and also bacterial infections, such as E. coli infections.

In another embodiment, the anti-viral-hemorrhagic agent is a tumor necrosis factor (TNF) inhibitor, including an anti-TNF antibody, an anti-TNF receptor antibody, a TNF receptor antagonist, a TNF production inhibitor, a TNF receptor production inhibitor or a TNF releasing inhibitor. In another exemplary embodiment, the anti-viral-hemorrhagic agent is an anti-viral vaccine, an anti-viral antibody, a viral-activated immune cell or a viral-activated immune serum. Any specific examples of the IL-1 inhibitor, the TNF inhibitor, the anti-viral vaccines, the anti-viral antibodies, the viral-activated immune cells or the viral-activated serum can be used in the combinational therapy.

The tetracycline compound(s) and/or the anti-viral-hemorrhagic agent(s) can be used alone or in combination with other known therapeutic agents or techniques (including chemotherapeutics, radioprotectants and radiotherapeutics) to either improve the quality of life of the patient, or to treat the disease, such as viral hemorrhagic diseases or disorders. For example, the tetracycline compound(s) and/or the anti-viral-hemorrhagic agent(s) can be used before, during or after radiation treatment.

F. Viral Hemorrhagic Diseases or Disorders and Diagnosis Thereof

The methods and compositions provided herein are particularly suited for treatment of viral hemorrhagic diseases. To effectively employ such methods, proper diagnosis is recommended. Hence following is a list of exemplary hemorrhagic diseases, the causative agents and methods of diagnosis.

Examples of the viral hemorrhagic diseases or disorders that can be treated by the present methods include, but not limited to, viral hemorrhagic disease caused by infection with Bunyaviridae, a Filoviridae, a Flaviviridae, or an Arenaviridae virus.

1. Bunyaviridae Virus Infection

Examples of Bunyaviridae viruses include bunyavirus (Bunyamwera, Bwamba, California, Capim, Guama, phlebovirus koongol, patois, simbu and tete viruses), sandfly fever virus, Rift Valley fever virus of sheep and ruminants, Nairovirus, Crimean-Congo hemorrhagic fever virus, Uukuvirus, Uukuniemi virus, Hantaan virus and Korean hemorrhagic fever virus (see, e.g., U.S. Pat. No. 5,786,342). Of particular interest is treatment of Crimean-Congo hemorrhagic fever virus, Hantaan virus and Korean hemorrhagic fever virus infections, particularly, Hantaan virus. Specific strains of Hantaan virus include 76-118 strain (Avsic-Zupanc, et al., Am. J. Trop. Med. Hyg., 51(4):393-400 (1994); Gu, et al., Chin. Med. J. (Engl)., 103(6):455-9 (1990); Miyamoto, et al., Kansenshogaku Zasshi., 61f(6):633-8 (1987 Jun); and Miyamoto, et al., Kansenshogaku Zasshi., 61(6):639-44 (1987 Jun)) and WKM strain (Yoo, et al., Microbiol. Immunol., 37(7):557-62 (1993); and Yoshimatsu, et al., J. Gen. Virol., 77(4):695-704 (1996 Apr)).

Bunyaviridae virus infection, and particularly Hantaan virus infection, can be diagnosed by any methods known in the art according to clinical, immunological or molecular criteria. Any known immunological methods can be used in the diagnosis of Bunyaviridae or Hantaan virus infection (see e.g., Current Protocols in Immunology (Ed. Coligan et al.) John Wiley & Sons, Inc., 1997); Sambrook et al., Molecular Cloning: A Laboratory Manual (2nd Ed.), Cold Spring Harbor Laboratory Press, 1989)). Such methods are known (see, e.g., Burkhardt, et al., Fortschr. Med., 111 (33):528-9 (1993) and van Ypersele de Strihou, et al., Lancet, 2(8365-66):1493 (1983)). Antibody-based or antigen-based immunological methods include immunoprecipitation, Western blotting, dot blotting and in situ immuno-detection methods such as immunofluorescence can be used. In a specific embodiment, anti-Bunyaviridae virus or anti-Hantaan virus antibodies described herein can be used in the immunodiagnosis.

Nucleotide-sequence based molecular methods include nucleotide sequencing, nucleotide hybridization, polymerase chain reaction (PCR), especially reverse-transcriptase polymerase chain reaction (RT-PCR) can be used. Hantaan virus nucleotide fragments with all or portions of the following Genbank Accession Nos. can be used in the nucleotide-sequence based molecular diagnosing methods: AF035831, X95077, D25531, D25528-D25530, D25532-D25533, U71369-U71372, U71281-U71283, X55901, S74081, S67430, U38911, U38910, Y00386, U38177, U37768, U37729, M14626, M57637, M14627, M57432 and L08753.

2. Filoviridae Virus Infection

Filoviruses are classified in the order Mononegavirales (Pringle C. R., Arch. Virol., 117:137-140 (1991)), which also contains the nonsegmented negative-strand RNA virus families Paramyxoviradae, Rhabdoviridae, and Bornaviridae. Members of the family Filoviridae includes Marburg virus, a unique agent without known subtypes, and Ebola virus, which has four subtypes (Zaire, Sudan, Reston, and Ivory Coast) (Feldmann and Slenczka Klenk, Arch. Virol. 11 (Suppl):77-100 (1996); LeGuenno B., et al., Lancet, 345:1271-127 (1995); Pringle C. R., Arch. Virol., 117:137-140 (1991)). Specific strains of ebola virus include Zaire strain (Jaax, et al., Lancet, 346(8991-8992):1669-71 (1995), Andromeda strain (Johnson, Ann. Intern. Med., 91(1):117-9 (1979), Gabon 94 strain (Prehaud, et al., J. Gen. Virol., 79(11):2565-72 (1998) and Sudan, Reston, and Ivory Coast strains (Feldmann and Slenczka Klenk, Arch. Virol. 11 (Suppl):77-100 (1996); LeGuenno B., et al., Lancet, 345:1271-127 (1995); Pringle C. R., Arch. Virol., 117:137-140 (1991)).

Filoviruses are enveloped, nonsegmented negative-stranded RNA viruses. The two species, Marburg and Ebola virus, are serologically, biochemically, and genetically distinct. Classification, virion morphology and structure, genomic organization and diagnosis are described in detail in Beer et al., Naturwissenschaften, 86:8-17 (1999), Springer-Verlag 1999. Marburg and Ebola viruses are pleomorphic particles that vary greatly in length, but the unit length associated with peak infectivity is 790 nm for Marburg virus and 970 nm for Ebola virus (Regnery et al., J. Virol., 36:465-469 (1980)). The virions appear as either long filamentous (and sometimes branched) forms or in shorter U-shaped, 6-shaped (mace-shaped), or circular (ring) configurations (Murphy et al., Paltyn S. R. (ed) Ebola virus hemorrhagic fever, Elsevier/North-Holland, Amsterdam, pp. 61-82 (1978); Peters et al., Martini and Siegert (eds) Marburg virus disease, Springer, Berlin Heidelberg, New York, pp. 68-83 (1971)). Virions have a uniform diameter of 80 nm and a density of 1.14 g/ml. They are composed of a helical nucleocapsid, a closely apposed envelope derived from the host cell plasma membrane, and a surface projection layer composed of trimers of viral glycoportein (GP) (Feldmann et al. (1991) Virology 182:353-356). All filoviruses contain one molecule of noninfectious, linear, negative-sense, single-stranded RNA with a Mᵣ of 4.2×10⁶, constituting 1.1% of the virion mass (Kiley M. P et al. (1988) J. Gen. Virol. 69:1957-1567 (1988); Regnery et al. (1980) J. Virol. 36:465-469).

The nonsegmented negative-strand RNA genomes of filoviruses show the gene arrangement 3′-NP-VP35-VP40-GP-VP30-VP24-L-5′ with a total molecular length of approximately 19 kb (Table 2).

TABLE 2
Filoviral proteins and functions

| | Virus | Encoding | | |
|---|---|---|---|---|
| Designation | type | gene | Localization | Function |
| NP | MBG/EBO | 1 | Ribonucleocapsid | Encapsidation |
| | | | complex | |
| VP35 | MBG/EBO | 2 | Ribonucleocapsid | Phosphoprotein |
| | | | complex | analogue |
| VP40 | MBG/EBO | 3 | Membrane- | Matrix protein |
| | | | association | |
| GP | MBG/EBO | 4 | Surface | Receptor binding, fusion |
| | | | (transmembrane | |
| | | | protein) | |
| VP30 | MBG/EBO | 5 | Ribonucleocapsid | Encapsidation, |
| | | | complex | necessary for |
| | | | | transcription and |
| | | | | replication |
| VP24 | MBG/EBO | 6 | Membrane- | Unknown (minor matrix |
| | | | association | protein, uncoating) |
| L | MBG/EBO | 7 | Ribonucleocapsid | RNA-dependent |
| | | | complex | |
| sGP | EBO | 4 | Nonstructural, | Unknown |
| | | | secreted | |

NP nucleoprotein; VP virion structural protein; GP glycoprotein; L large protein (polymerase); sGP small glycoprotein; MBG type Marburg filoviruses; EBO type Ebola filoviruses Modified after Feldmann et al., Archives of Virology, 1996.

Filoviridae virus infection, and particularly ebola and Marburg virus infection, can be diagnosed by any methods known in the art according to clinical, immunological or molecular criteria (see, e.g., Sambrook et al., Molecular Cloning: A Laboratory Manual (2nd Ed.), Cold Spring Harbor Laboratory Press, 1989). Antibody-based or antigen-based immunological methods include immunoprecipitation, Western blotting, dot blotting and in situ immuno-detection methods such as immunofluorescence can be used. In a specific embodiment, anti-Filoviridae virus or anti-ebola and anti-Marburg virus antibodies, such as those described herein, can be used in the diagnosis of Bunyaviridae or Hantaan virus infection (see, e.g., Current Protocols in Immunology (Ed. Coligan et al.) John Wiley & Sons, Inc., 1997).

Nucleotide-sequence based molecular methods include nucleotide sequencing, nucleotide hybridization, polymerase chain reaction (PCR), especially reverse-transcriptase polymerase chain reaction (RT-PCR) can be used. In a specific embodiment, the ebola virus nucleotide sequences with the following Genbank Accession Nos. can be used in the nucleotide-sequence based molecular diagnosing methods: AF086833, U77384-U77385, U8116-U23417, U23187, U23152, U23069, AF034645, AF054908, X67110, L 11365, U28077, U28134, U28006, U31033, U23458, X61274, J04337 and M33062. In another specific embodiment, the Marburg virus nucleotide sequences with the following Genbank Accession Nos. can be used in the nucleotide-sequence based molecular diagnosing methods: AF005730-AF005735, Z12132, Z29337, X64405-X64406, X68493-X68495, M72714, M92834 and M36065.

Reverse transcriptase polymerase chain reaction is one of the most powerful tools of diagnosis of filovirus infection (Volchkov V., et al., Virology, 232:139-144 (1997)). Antibodies to filovirus can be detected by immunofluorescence assays using acetone-fixed virus-infected cells inactivated by A-radiation └Johnson et al., Trans. R. Soc. Trop. Med. Hyg., 76:307-310 (1982); Johnson et al., Trans. R. Soc. Trop. Med. Hyg., 77:731-733 (1983)), which should not be used under field conditions. An enzyme-linked immunosorbent assay using a mild detergent extract of infected Vero cells adsorbed to plastic plates has been shown to be more reliable (Ksiazek, Lab. Anim., 20:34-46 (1991)) under such conditions.

Vero cells are readily used for the isolation and propagation of fresh and laboratory passaged strains of the viruses. MA-104 cells and SW13 cells have also been successful in primary filovirus isolation (McCormick et al., J. Infect. Dis., 147:264-267 (1983)). In some circumstances primary isolation in guinea pigs (for Marburg virus) or suckling mice (for Ebola virus) may be required.

A western blot method has been standardized for the diagnosis of filovirus infections [Elliott et al., J. Virol. Methods, 43:85-89 (1993)). Solid-phase indirect enzyme-immunoassay (SPEIA) has been used to detect Lassa and Ebola virus antigens and antibodies using horseradish peroxidase-labeled antispecific globulins (Ivanov etal. (1985) Vopr Virusol. 31(2):186-190). Immunohistochemistry (IHC) testing of formalin-fixed postmortem skin specimens can also be performed (see, e.g., Zaki et al. (1999) J. Infect. Dis. 179(Suppl1):S36-47).

3. Flaviviridae Virus Infection

All members of the Flaviviridae family share common morphologic characteristics, genome structure, and replication and translation strategies (see, e.g., Kautner, et al., J. Pediatr., 131:516-524 (1997)). Examples of Flaviviridae viruses include flavivirus, Brazilian encephalitis virus, Bussuquara virus, Dengue virus, iiheus virus, Israel turkey meningoencephalitis virus, Japanese B encephalitis virus, Kunjin virus, Kyasanur forest disease virus, Langat virus, Louping ill virus, Modoc virus, Murray valley encephalitis virus, Ntaya virus, omsk hemorrhagic fever virus, powassan virus, St. Louis encephalitis virus, spondwnei virus, tick-borne encephalitis, Uganda S virus, US bat salivary gland virus, wesselsbron virus, West Nile fever virus, yellow fever virus, Zika virus, European tick-borne encephalitis, Far Eastern tick-borne encephalitis virus and Russian tick-borne encephalitis (U.S. Pat. No. 5,786,342). Preferably, the Dengue virus to be treated is a Dengue type 1, Dengue type 2, Dengue type 3 or Dengue type 4 virus. Specific Dengue type 1 virus strains include Singapore strain S275/90 (Fu, et al., Virology, 188(2):953-8 (1992)), Western Pacific strain (Puri, et al., Virus Genes, 17(1):85-8 (1998)) and Mochizuki strain (Zulkarnain, et al., Micobiol. Immunol., 38(7):581-5 (1994)). Specific Dengue type 2 virus strains include Brazilian strain (Barth, et al., Mem. Inst. Oswaldo. Cruz., 86(1):123-4 (1991)), New Guinea C strain (Biedrzycka, et al., J. Gen. Virol., 68(5):1317-26 (1987); Irie, et al., Gene, 75(2):197-211 (189); Kapoor, et al., Gene, 162(2):175-80 (1995); Price, et al., Am. J. Trop. Med. Hyg., 22(1):92-9 (1973)), strain 16681 (Kinney, et al., Virology, 230(2):300-8 (1997)), strain PR-159 (Leblois, et al., Nucleic Acids Res., 21(7):1668 (1993)), Cuban A15 strain (Pupo-Antunez, et al., Hybridoma., 16(4):347-53 (1997)) and Mexican strain (Sanchez, et al., J. Gen. Virol., 77(10):2541-5 (1996)). Hence, the family Flaviviridae includes human pathogens, Dengue viruses, the Japanese encephalitis virus and yellow fever virus.

Four Dengue virus serotypes and various “biotypes” can be differentiated. Mature Dengue virus particles have a single-stranded ribonucleic acid genome surrounded by an approximately icosahedral nucleocapsid with a diameter of 30 nm. The nucleocapsid is covered by a lipid envelope of 10 nm thickness derived from host cell membranes and contains the envelope and membrane proteins (Westaway et al., Flaviridiac. Intervirology, 24:183-92 (1985)).

The viral genome of approximately 11 kb is infectious, has a messenger-like positive polarity, and can be translated in vitro. The 5′ end of the RNA has a type I cap structure but lacks a poly A tail at the 3′ end (Rice et al., Science, 229:726-33 (1985); Hahnet al., Virology, 162:167-80 (1988); Irie et al., Gene, 74:197-211 (1989)). It contains a single open reading frame of about 10,000 nucleotides encoding three structural and seven nonstructural proteins. The gene order is 5′-C-prM(M)-E-NSI-NS2A-NS2B-NS3-NS4A-NS4B-NS5. The proteins are synthesized as a polyprotein of about 3000 aminoacids that is processed cotranslationally and posttranslationally by viral and host proteases (Biedrzycka et al., J. Gen. Virol., 1987, 68:1317-26; Mackow et al., J. Gen. Virol., 1987, 69:23-4; Speight et al., Virology, 1987, 159(2):217-28; Chambers et al., Virology, 1989, 169:100-9; Markoff etal., J. Virol., 1989, 63:3345-52; Preugschar et al., J. Virol., 1990, 64:4364-74; Falgout et al., J. Virol., 1991, 65:2467-75; Preugschat et al., J. Virol., 1991, 65:4749-58; Preugschat F., et al., Virology, 1991, 185:689-97; Cahour et al., J. Virol., 1992, 66:1535-42).

The structural proteins include a capsid protein rich in arginine and lysine residues and a nonglycosylated prM protein produced from a glycosylated precursor in a late step of virus maturation (Rice et al., Science, 1985, 229:726-33; Hahn et al., Virology 1988, 162:167-80; Deubel et al., J. Virol. Methods, 1990, 30:41-54;Randolph et al., Virology 1990, 174:450-8). The major structural envelope protein is involved in the main biologic functions of the virus particle such as cell tropism, acid-catalyzed membrane fusion, and the induction of hemagglutination-inhibiting, neutralizing, and protective antibodies (Depres et al., Virology, 1993, 196:209-219).

The first nonstructural protein is NSI, a glycoprotein with a function in the virus life cycle that is unknown (Schlesinger et al., J. Immunol., 1985, 135:2805-9). NS1 proteins are detected in high titers in patients with secondary Dengue infections, but are rarely found in primary infections (Kuno et al., J. Med. Virol., 1990, 32:102-8). The NS2 region codes for two proteins (NS2A and NS2B) that are thought to be implicated in polyprotein processing, whereas NS3 is probably the viral proteinase that functions in the cytosol (Preugschat et al., Virology, 1991, 185:689-97; Cahour et al., J. Virol., 1992, 66:1535-42; Falgout et al., J. Virol., 1989, 63:1852-60). The NS4 region codes for two small hydrophobic proteins that seem to be involved in the establishment of the membrane bound RNA replication complex. The protein encoded by the NS5 gene has a molecular weight of 105,000, is the most conserved flavivirus protein and is the virus-encoded RNA-dependent RNA polymerase.

Flaviviridae virus infection, and particularly Dengue virus infection, can be diagnosed by any methods known in the art according to clinical, immunological or molecular criteria. Any known immunological methods can be used in the diagnosis of Flaviviridae or Dengue virus infection (see Current Protocols in Immunology (Ed. Coligan et al.) John Wiley & Sons, Inc., 1997). Antibody-based or antigen-based immunological methods include immunoprecipitation, Western blotting, dot blotting and in situ immuno-detection methods such as immunofluorescence can be used. Antibodies described herein can be used in the immunodiagnosis.

Any known molecular methods can be used in the diagnosis of Flaviviridae or Dengue infection (Sambrook et al., Molecular Cloning: A Laboratory Manual (2nd Ed.), Cold Spring Harbor Laboratory Press, 1989). Nucleotide-sequence based molecular methods include nucleotide sequencing, nucleotide hybridization, polymerase chain reaction (PCR), especially reverse-transcriptase polymerase chain reaction (RT-PCR) can be used. Dengue virus nucleotide fragments containing all or portions of sequences with the following Genbank Accession Nos. can be used in the nucleotide-sequence based molecular diagnosing methods: E06832, D10514, D10513, X70952.

The diagnosis of Dengue relies in most case on clinical judgment because only a few major centers have the facilities and means to perform and verify the clinical impression. Diagnostic criteria for DHS based on clinical observations have been proposed by the World Health Organization and should be used to avoid over-diagnosis (World Health Organization. Dengue hemorrhagic fever: diagnosis, treatment and control, Geneva, WHO, 1986). Clinical criteria for diagnosis are as follows: (1) fever; (2) hemorrhagic manifestations, including at least a positive tourniquet test result and a major or minor bleeding phenomenon; (3) hepatic enlargement; (4) shock (high pulse rate and narrowing of the pulse pressure to 20 mm Hg or less, or hypotension). The laboratory criteria include (5) thrombocytopenia (≦100,000/mm³), and (6) hemoconcentration (hematocrit increase ≧20%). Thrombocytopenia with concurrent high hematocrit levels differentiates DHF from classic DF.

A secondary Dengue infection is characterized by the rapid appearance of broadly cross-reactive antibodies. Hemagglutination inhibition titers of 1:20 in the acute-phase sample rise to ≧1:2560 in the convalescent phase sample. An antibody titer of ≧1:1280 in the acute-phase sample without a fourfold or greater increase in the second sample also is considered presumptive of recent infection. A less time-consuming method is a capture enzyme-linked immunosorbent assay that can detect specific anti-Dengue IgM in a single acute-phase sample (Lam et al., Southeast Asian, J. Trop. Med. Public Health, 1987, 18:532-8).

Commercial kits for the detection of specific IgG as well as IgM antibodies have become available. They are based on a dot enzyme assay or a nitrocellulose membrane-based capture format, respectively, and should be suitable for field research (Cardosa et al., J. Virol. Methods, 1988, 22:81-8; Cardosa et al., Southeast Asian, J. Trop. Med. Public Health, 1988, 19:591-4; Cardosa et al., Clin. Diagn. Virol., 1995, 3:343-50).

An alternative to virus isolation is the detection of viral RNA by reverse transcription polymerase chain reaction. There are various protocols available using different primers and template isolation (Deubel et al., J. Virol. Methods, 1990, 30:41-54; Henchal et al., Am. J. Trop. Med. Hyg., 1991, 45:418-28; Morita et al., J. Clin. Microbiol., 1991, 29:2107-10; Morita et al., J. Med. Virol., 1994, 44:54-8; Lanciotti et al., J. Clin. Microbiol., 1992, 30:545-51; Suk-Yin et al., Southeast Asian, J. Trop. Med. Public Health, 1994, 25:258-61; Seah et al., J. Virol. Methods, 1995, 51:193-200). Reverse transcription polymerase chain reaction coupled with hybridization with labeled serotype-specific probes can detect as few as 4 plaque-forming units per 100 μl serum and gives the best results early in the acute phase of the disease when Dengue antibodies are still low (Suk-Yin et al., Southeast Asian, J. Trop. Med. Public Health, 1994, 25:258-61). Less than 1 μl of serum can be sufficient for the detection of viral RNA (Chan et al., J. Virol. Methods, 1994, 49:315-22).

4. Arenaviridae Virus Infection

Examples of Arenaviridae viruses include Junin virus, Lassa virus, Machupo virus, Pichinde virus, lymphocytic choriomeningitis virus, Lassa fever virus and arenavirus (U.S. Pat. No. 5,786,342). Preferably, the Arenaviridae viruses to be treated are Junin virus, Lassa virus, Machupo virus. Specific strains of Lassa virus include Josiah strain (Auperin, et al., Virology, 168(2):421-5 (1989); and Fidarov, et al., Vopr Virusol., 35(4):326-9 (1990) and Nigerian strain (Clegg, et al., Virus Res., 18(2-3):151-64 (1991)).

Arenaviridae virus infection, and particularly Lassa virus, Machupo virus, or Pichinde virus infection, can be diagnosed by any methods known in the art according to clinical, immunological or molecular criteria. Any known immunological methods can be used in the diagnosis of Arenaviridae virus infection, and particularly Lassa virus, Machupo virus, or Pichinde virus infection (see Current Protocols in Immunology (Ed. Coligan et al.) John Wiley & Sons, Inc., 1997). Antibody-based or antigen-based immunological methods include immuniprecipitation, Western blotting, dot blotting and in situ immuno-detection methods such as immunofluorescence can be used. In a specific embodiment, anti-Arenaviridae virus or anti-Lassa virus, anti-Machupo virus and anti-Pichinde virus antibodies known to those of skill art in the or described herein can be used in the immunodiagnosis.

Any known molecular methods can be used in the diagnosis of Arenaviridae virus infection, and particularly Lassa virus, Machupo virus, or Pichinde virus infection (Sambrook et al., Molecular Cloning: A Laboratory Manual (2nd Ed.), Cold Spring Harbor Laboratory Press, 1989); see also, Sarrat, et al., Bull Soc Pathol Exot Filiales., 65(5):642-50 (1972) (Histopathological diagnosis of hepatitis due to Lassa virus); and Trappier, et al., Am. J. Trop. Med. Hyg., 49(2):214-21 (1993) (Evaluation of the polymerase chain reaction for diagnosis of Lassa virus infection)).

Nucleotide-sequence based molecular methods include nucleotide sequencing, nucleotide hybridization, polymerase chain reaction (PCR), especially reverse-transcriptase polymerase chain reaction (RT-PCR) can be used. Lassa virus nucleic acid fragments containing sequences from the following Genbank Accession Nos. can be used in the nucleotide-sequence based molecular diagnosing methods: U80004, U73034-U73035, U63094, X52400, J04324, K03362 and M15076. Machupo virus nucleic acid fragments containing sequences from the following Genbank Accession Nos. can be used in the nucleotide-sequence based molecular diagnosing methods: X62616.

G. Examples

The following examples are included for illustrative purposes only and are not intended to limit the scope of the invention.

### EXAMPLE 1

### Treatment of Marburg and Lassa Virus Infection

a. Experimental Protocals

(1) Virus

Marburg virus strain Popp used in the following experiments was received from the Belarussian Research Institute of Epidemiology and Microbiology (Minsk, Belarussia). All work with infectious virus was performed in the maximum-containment biosafety level-4 (BSL-4) facility of the State Scientific Center of Virology and Biotechnology (“Vector”) (Koltsovo, Russia). This virus was amplified in Vero E 6 cells and the supernatant was collected to produce stocks. This stock virus suspension (2×10⁷ PFU/ml) was stored at −70° C.

Lassa virus strain Josiah used in the following experiments was received from Belarussian Research Institute of Epidemiology and Microbiology (Minsk, Belarussia). This mouse-adapted Lassa virus was passaged once in Vero E6 cells and 3 times passaged in mice by intracerebrally challenge. This mouse-adapted Lassa virus stock was collected and stored at −70° C. This stock contained 10⁶ PFU ml (or 10⁵ LD₅₀ by inoculation challenge of 4-week old BALB/c mice).

(2) Animals

Outbred Hartly guinea-pigs of 200-220 grams were used in the experiments with Marburg virus. Four-week old BALB/c mice (haplotype H-2d) were used in the experiments with Lassa virus.

The animals were received from the vivarium of SRC VB “Vector” and kept at a standard ration. To ensure that the animals (guinea-pigs and mice) were spared of unnecessary pain and discomfort, standard anesthesia methods were used. A single dose of ketamine/xylazine via intramuscular injection in the posterior region of the hind leg was administered to the animals.

(3) PCR

RT-PCR procedure for Lassa virus detection was performed as described in Demby et al., J. Clinical Microbiology, 32:2898-2903 (1994) and for Marburg virus detection as described in Ignatyev et al., In: Berg D. A. (ed) Proceedings of the 1996 ERDEC scientific conference on chemical and biological defense research, Nov. 19-22, 1996, pp. 323-330 (1996).

b. Treatment of Marburg Virus Infection

Animals were divided into 11 groups, each containing 6 animals:

1. Animals of the first group serve as virus controls, ie., were infected with the virus but were not given therapeutic or prophylactic or any treatment agents.

2ₜ. Animals of the second group (T) were given 1 ml of Tetracycline-Hcl (Belmedpreparats Ltd., Russia) solution (58 mg/kg) intramuscularly from 10 days before virus injection until seventh day after virus injection daily.

2_{d}. Animals of the second group (D) were given 1 ml of Doxycycline solution (Belmedpreparats Ltd., Russia) (58 mg/kg) intramuscularly from 10 days before virus injection until seventh day after injection daily.

3ₜ. Animals of the third group (T) were given 1 ml of Tetracycline-Hcl solution (58 mg/kg) intramuscularly from 5 days before virus injection until seventh day after injection daily.

3_{d}. Animals of the third group (D) were given 1 ml Doxycycline solution (58 mg/kg) intramuscularly from 5 days before virus injection until seventh day after injection daily.

4ₜ. Animals of the fourth group (T) were given 1 ml of Tetracycline-Hcl solution (58 mg/kg) intramuscularly from the third day after virus injection until seventh day after virus injection daily.

4_{d}. Animals of the fourth group (d) were given 1 ml Doxycycline solution (58 mg/kg) intramuscularly from the third day after virus injection until seventh day after virus injection daily.

5ₜ. Animals of the fifth group (T) serve as the Tetracycline controls, i.e., were given Tetracycline-Hcl solution (58 mg/kg) intramuscularly during the 17 day period without virus injection.

5_{d}. Animals of the fifth group (d) serve as the Doxycycline controls, i.e., were given Doxycycline solution (58 mg/kg) intramuscularly during the 17 day period without virus injection.

Animals of the above groups were parenterally infected with Marburg virus at a dose of 5LD₅₀ on day “0”. The virus was detected by RT-PCR on the third day after infection.

As seen in Table 3, tetracycline and doxycycline are not toxic to control groups (5T, 5D). Using tetracycline and doxycycline prophylactically does not improve survival rate of the animals (2T, 2D, 3T and 3D). In fact, the mean time to death (m.t.d.) of these groups is shorter than that of the virus control group (1). In contrast, using tetracycline and doxycycline therapeutically increases survival rate of the animals because 2 animals from the group 4T and 4D, respectively, survived the otherwise lethal infection. In addition, the m.t.d. of groups 4T and 4D is slightly longer than that of the virus control group (1).

TABLE 3
Tetracycline and Doxycycline by experimental Marburg-virus infection

| | guinea pigs | | | |
|---|---|---|---|---|
| Group | (total) | survival | m.t.d. | |
| 1 | 6 | 0 | 8.2 | (control virus) |
| 2T | 6 | 0 | 8.06 | |
| 2D | 6 | 0 | 7.69 | |
| 3T | 6 | 0 | 7.91 | |
| 3D | 6 | 0 | 7.6 | |
| 4T | 6 | 2 | 8.75 | |
| 4D | 6 | 2 | 8.54 | |
| 5T | 6 | 6 | — | (tetracycline |
| | | | | control) |
| 5D | 6 | 6 | — | (doxycycline |
| | | | | control) |

m.t.d. - mean time to death

m.t.d.—mean time to death

C. Treatment of Lassa Virus Infection

Animals were divided into the following groups, each containing 20 mice:

1. Animals of the first group were infected with Lassa virus without any tetracycline or doxycycline treatment.

2. Animals of the second group were given 0.2 ml of Tetracycline-HCl solution (58 mg/kg) from the third day until 7th day after virus injection (every day).

3. Animals of the third group were given 0.2 ml of Doxycycline-HCl solution (58 mg/kg) form the third day until 7th day after virus injection (every day).

4. Animals of the fourth group were given Tetracycline-HCl solution during a 7 day period without viral infection.

5. Animals of the fifth group were given Doxycycline-HCl solution during a 7 day period without viral infection.

Animals of groups 1-3 were infected intracerebrally with Lassa virus at a dose of 10 PFU/0.03 ml on day “0”. The virus was detected by RT-PCR on the third day after infection.

As seen in Table 4, tetracycline and doxycycline are not toxic to control groups (1). Using tetracycline and doxycycline therapeutically increases survival rate of the animals because Group 2 and 3 have higher survival rates than Group 1 (P<0.01). In addition, the m.t.d. of groups 2-3 is slightly longer than that of Group 1.

Levels of IL-1, IL-1Ra, TNF and soluble TNF receptor (sTNFR) were monitored in the Lassa virus control animals (Table 5) and tetracycline or doxycycline treated animals (Table 6) by ELISA using the ELISA kits or antibodies from R&D Systems, Inc. (U.S.A.). The ratio of IL-1/IL-1Ra in virus control animals (Table 5) increased dramatically to about 20 fold of the base level (Day 9) as the infection progressed and then returned to the base level (Day 21). In contrast, the ratio of IL-1/IL-1Ra in tetracycline or doxycycline treated animals (Table 6) increased to only about 5 fold of the base level (Day 3) and then returned to the base level (Day 21). Based upon the kinetics of the IL-1/IL-Ra ratio and sTNFr, treatment with a tetracycline compound appears to abort or limit infection.

TABLE 4
Tetracycline and Doxycycline for experimental Lassa - virus infection

| | | Mice | | |
|---|---|---|---|---|
| Group | Total | death | survival | m.t.d. |
| 1 (virus control) | 20 | 12 | 8 | 8.92 |
| 2 (doxycycline treatment) | 20 | 6 | 14 | 9.09 |
| 3 (tetracycline treatment) | 20 | 4 | 16 | 9.43 |
| 4 (doxycycline control) | 20 | 0 | 20 | n.d. |
| 5 (tetracycline control) | 20 | 0 | 20 | n.d. |

m.t.d. - mean time to death
n.d. - no detection

m.t.d.—mean time to death n.d.—no detection

TABLE 5
IL-1, IL-1Ra, TNF and sTNFr production in control animals
CONTROL Lassa VIRUS (pg/ml) (Survival 8 from 20)

| DAYS | IL-1 | IL-IRA | IL-1/IL-IRA | TNF | sTNFr |
|---|---|---|---|---|---|
| 0 | 1.9 | 51 | 0.037 | 1.56 | 12.6 |
| 1 | 7.6 | 66 | 0.115 | 4.8 | 16.4 |
| 3 | 21.84 | 120 | 0.182 | 22.6 | 25 |
| 5 | 41.5 | 130 | 0.319 | 22.8 | 25 |
| 7 | 47.88 | 121 | 0.395 | 23.4 | 25 |
| 9 | 49.92 | 66 | 0.756 | 22.6 | 25 |

m.t.d. 8.92

| | | | | | |
|---|---|---|---|---|---|
| 15 | 22.15 | 121 | 0.183 | 16.4 | 100 |
| 21 | 3.2 | 63 | 0.050 | 2.4 | 18.2 |

TABLE 6
IL-1, IL-1ra, TNF and sTNFr production in Lassa virus infected animals

| | Doxycycline | | | | | Tetracycline | | | | |
|---|---|---|---|---|---|---|---|---|---|---|
| | | | IL-1/IL- | | | | | IL-1/IL- | | |
| DAYS | IL-1 | IL-IRA | IRA | TNF | TNFR | IL-1 | IL-IRA | IRA | TNF | sTNFR |
| 0 | 1.9 | 51 | 0.037 | 1.56 | 12.6 | 1.9 | 51 | 0.037 | 1.56 | 12 |
| 1 | 7.6 | 66 | 0.115 | 4.8 | 16.4 | 7.6 | 66 | 0.115 | 4.8 | 16 |
| 3 | 21.84 | 120 | 0.182 | 22.6 | 25 | 21.84 | 120 | 0.182 | 22.6 | 2 |
| 5 | 38.3 | 280 | 0.136 | 20.4 | 52 | 19.4 | 180 | 0.107 | 26.4 | 40 |
| 7 | 31.2 | 500 | 0.060 | 17.16 | 751 | 12.48 | 200 | 0.062 | 20.28 | 100 |
| 9 | 16.6 | 690 | 0.024 | 16.2 | 721 | 10.2 | 520 | 0.019 | 17.2 | 120 |
| 15 | 12.48 | 175 | 0.073 | 14.04 | 20 | 7.8 | 84 | 0.091 | 14.82 | 50 |
| 21 | 2.6 | 56 | 0.046 | 2.1 | 13.8 | 2.4 | 54 | 0.044 | 2.0 | 16 |

| | |
|---|---|
| m.t.d. 9.09 | m.t.d. 9.43 |
| survival 14 (20) | survival 16 (20) |
| 70% | 80% |

### EXAMPLE 2

### Treatment of Dengue Virus Infection

a. Experimental Protocols

(1) Virus

Dengue virus, type 2 was used in the following experiments. All work with infectious virus was performed in the maximum-containment biosafety level-3 (BSL-3) facility of the “Vector”. This virus was amplified in the brain of succlik mice (inbred BALB/c mice from Vector) and was collected to produce stocks. This stock virus suspension was stored at −40° C., containing 6.8 lg LD₅₀/ml (in mice BALB/c by intraperitoneal challenge).

(2) Animals

4-week old BALB/c mice (haplotype H-2d) were used in the experiments with Dengue virus infection. Mice weigh 12-14 grams. The animals were received from SRC VB “Vector” and kept at a standard ration.

(3) RT-PCR Procedure

The virus detection was provided by PCR-method. Primers for Dengue virus type 2 detection are upper 5′AATATGCTGAAACGCGAGAGAAACCG (position 136-161 of the Dengue virus RNA SEQ ID No. 23 and lower 5′AAGGAACGCCACCAAGGCCATG (position 237-258) SEQ ID NO. 24.

RNA was extracted from serums of infected animals (mice) using the RNeasy Kit (Quigen, Germany). For RT-PCR, Titan kits (Behringer, Germany) were used. Reverse transcription was conducted at 42° C. for 60′ followed by 40 amplification cycles at 94° C. for 30″, 55° C. for 1′, and 68° C. for 2′ with a final extension at 68° C. for 7 mins. Amplification was conducted in 0.2 ml tubes with a model BIS-105M thermocycler (Russia).

b. Treatment of Dengue Virus Infection

Group 1

The animals of this group (60 animals) were given Doxycycline solution (58 mg/kg) intramuscularly every day for 4 days. From the first day, sera were taken from mice daily to detect concentration of IL-1, TNF, IL-1RA and sTNFr (Table 7).

Group 2

Animals of the second group are virus controls, i.e., were infected with the Dengue virus without doxycycline treatment. The virus detection was provided by PCR-method on the second day after infection. From the first day after infection, sera were taken from mice daily to detect concentration of IL-1, TNF, IL-1RA and sTNFr (Table 8).

Group 3

The animals from this group were given 0.2 ml of Doxycycline solution (58 mg/kg) intramuscularly from the second day after virus injection till the fifth day daily. The virus detection was provided by PCR-method on the second day after infection. From the first day after infection, sera were taken from mice daily to detect concentrations of IL-1, TNF, IL-1RA and sTNFr (Table 9).

Group 4

The animals from this group were given 0.2 ml of doxycycline solution (58 mg/kg) intramuscularly from the third day after virus injection till the fifth day daily. The virus detection was provided by PCR-method on the second day after the infection. From the first day after infection, sera were taken from mice daily to detect concentration of IL-1, TNF, IL-1RA and sTNFr (Table 10).

Group 5

The animals from this group were given, intravenously daily from the second day after infection till the sixth day, 0.3 ml of the serum collected from the animals of the group 1 on the first day after those animals were treated with doxycycline. In this volume, the Serum collected from the animals of group 1 contain 6.6 pg IL-1, 60 pg IL-1ra, 1.5 pg TNF and 25 pg sTNFr. The virus detection was provided by PCR-method on the second day after infection. From the first day after infection, the sera were taken from the mice of group 5 to detect concentration of IL-1, TNF, IL-1RA and sTNFr (Table 11).

Group 6

The animals from this group were given, intravenously daily from the second day after infection till the sixth day, 0.3 ml of the serum collected from the animals of the group 1 on the second day after those animals were treated with doxycycline. In this volume, the Serum collected from the animals of group 1 contain 6 pg IL-1, 20 pg IL-1ra, 5.5 pg TNF and 12 pg sTNFr. The virus detection was provided by PCR method on the second day after animals infection. From the first day after infection, sera were taken from the mice of Group 6 daily to detect concentration of IL-1, TNF, IL-1RA and sTNFr (Table 12).

C. Results and Discussion

As seen in Table 7, injection of doxycycline to the uninfected mice increases production of the cytokines and their soluble receptors. It is noteworthy that this response, i.e., increased production of cytokines and their soluble receptors, to the first doxycycline injection was higher than to the second and the third doxycycline injection. This difference signifies development of the refractory period in mice on the second and the third day after the injection of doxycycline. Therefore, multiple injections of doxycycline to the uninfected mice does not keep high concentrations of the soluble cytokine receptors in their sera. Also, the survival rate for group 5, which received serum containing 60 pg of IL-1Ra was higher than that in group 6 in which the IL-1Ra level was 20 pg.

The experiments using BALB/c (haplotype H-2d) and C57BI/6 (H-2b) mice show that the dosage of Dengue virus of 10-10,000 LD₅₀ is absolutely lethal (100%) after intraperitoneal challenge to these mice weighing 12-14 grams.

In the experiments described below, BALB/c mice weighing 12-14 grams were used. These mice died toward the end of the fifth day after the infection with the dose of Dengue virus 100 LD₅₀. In the sera of animals from Group 2 (virus control group), the concentration of IL-1 increases during the development of the infection more significantly than the concentration of IL-1RA (Table 8). The large excess of IL-1 over IL-1RA manifests in the ratio of IL-1/IL-1RA.

These experiments show the importance of detecting the ratio IL-1/IL-1RA in prognosis of the development of the disease caused by the Dengue virus infection. The change in the ratio of TNF to sTNFr during the course of Dengue virus infection is analogous to that of the ratio of IL-1 to IL-1RA. Overall, the concetration of these two cytokines increases more significantly than that of their respective receptors during the course of the infection. The concentration of TNF increased 500 times on the day of death but the concentration of sTNFr only increased 4 times. In addition, the ratio of TNF/sTNFr, rather than the TNF concentration itself, is more significant for the resolution of Dengue virus infection.

TABLE 7
Level of IL-1, TNF, soluble receptors: IL-IRA and sTNF
after Doxycycline solution injection (Group 1)

| | | | | | | | |
|---|---|---|---|---|---|---|---|
| | | | IL-I/ | | | TNF/s | |
| | IL-1 | IL-IRA | IL-IRA | TNF | sTNFrI | TNFrI | |
| Days | (pg/ml) | (pg/ml) | (I) | (pg/ml) | (pg/ml) | (II) | I + II |

Before the injection

| | | | | | | | |
|---|---|---|---|---|---|---|---|
| 0 | 2.95 | 30 | 0.098 | 1.17 | 17 | 0.068 | 0.166 |

After the injection

| | | | | | | | |
|---|---|---|---|---|---|---|---|
| 1* | 20.62 | 180 | 0.115 | 4.68 | 85 | 0.072 | 0.187 |
| 2** | 17.43 | 60 | 0.291 | 17.55 | 38 | 0.462 | 0.753 |
| 3 | 17.48 | 80 | 0.219 | 9.36 | 35 | 0.267 | 0.486 |
| 4 | 17.93 | 175 | 0.102 | 8.19 | 38 | 0.216 | 0.318 |

*= Serum 1
**= Serum 2

TABLE 8
Level IL-1, TNF, soluble receptors: IL-1RA and sTNF
during of the experimental Dengue-virus infection (Group 2)

| | | | IL-I/IL- | | | | | |
|---|---|---|---|---|---|---|---|---|
| | IL-I | IL-IRA | IRA | TNF | sTNFrI | TNF/sTNF | | Survival/ |
| DAYS | (pg/ml) | (pg/ml) | (I) | (pg/ml) | (pg/ml) | (II) | I + II | dead |
| 0 | 2.95 | 30 | 0.098 | 1.17 | 17 | 0.068 | 0.166 | 10/0 |
| 1 | 10.6 | 70 | 0.151 | 8.19 | 32 | 0.256 | 0.407 | 10/0 |
| 2 | 16.8 | 65 | 0.258 | 26.9 | 37 | 0.727 | 0.985 | 10/0 |
| 3 | 26.7 | 70 | 0.381 | 35.1 | 45 | 0.780 | 1.161 | 10/0 |
| 4* | 32.76 | 78 | 0.420 | 51.6 | 45 | 1.147 | 1.567 | 8/2 |
| 5*# | 40.6 | 92 | 0.441 | 562.5 | 65 | 8.654 | 9.095 | 0/8 |

m.t.d.-4.76
*-blood samples taken from mice with clinical symptoms.
#-5 mice dies to the beginning of the fifth day and 3 mice to the end of this day.
m.t.d.-mean time of death

TABLE 9
Level of IL-1, TNF, soluble receptors; IL-1RA and sTNF during the
Doxycycline treatment (from the second day) of the experimental
Dengue-infection (type 2) (Group 3)

| | | | IL-1/ | | | | | |
|---|---|---|---|---|---|---|---|---|
| | IL-1 | IL-IRA | IL-IRA | TNF | sTNFrI | TNF/sTNF | | Survival/ |
| Days | (pg/ml) | (pg/ml) | (I) | (pg/ml) | (pg/ml) | (II) | I + II | dead |
| 0 | 2.95 | 30 | 0.083 | 1.17 | 17 | 0.068 | 0.151 | 10/0 |
| 1 | 10.6 | 70 | 0.151 | 8.19 | 32 | 0.256 | 0.407 | 10/0 |
| 2 | 16.8 | 65 | 0.258 | 26.9 | 37 | 0.727 | 0.985 | 10/0 |

the beginning of the treatment

| | | | | | | | | |
|---|---|---|---|---|---|---|---|---|
| 3 | 17.9 | 85 | 0.211 | 19.89 | 46 | 0.432 | 0.643 | 10/0 |
| 4 | 24.18 | 76 | 0.318 | 24.57 | 50 | 0.491 | 0.809 | 10/0 |
| 5 | 30.42 | 78 | 0.390 | 262.5 | 70 | 3.75 | 4.14 | 10/0 |
| 6 | n.d. | n.d. | n.d. | n.d. | n.d. | n.d. | n.d. | 0/10 |

m.t.d.-mean time to death—6 days
n.d.-no death

TABLE 10
Level of IL-1, TNF, soluble receptors; IL-1RA and sTNF during
the Doxycycline treatment (from the third day) of the
experimental Dengue (type 2) virus infection (Group 4)

| | IL-1 | | IL-1/ | TNF | | TNF/ | | Surviv- |
|---|---|---|---|---|---|---|---|---|
| | (pg/ | IL-IRA | IL-IRA | (pg/ | sTNFrI | sTNF | | al/ |
| Days | ml) | (pg/ml) | (I) | ml) | (pg/ml) | (II) | I + II | dead |
| 0 | 2.95 | 30 | 0.083 | 1.17 | 17 | 0.068 | 0.151 | 10/0 |
| 1 | 10.6 | 70 | 0.151 | 8.19 | 32 | 0.256 | 0.407 | 10/0 |
| 2 | 16.8 | 65 | 0.258 | 26.9 | 37 | 0.727 | 0.985 | 10/0 |
| 3 | 26.7 | 70 | 0.381 | 35.1 | 45 | 0.780 | 1.161 | 10/0 |

the beginning of the treatment

| | | | | | | | | |
|---|---|---|---|---|---|---|---|---|
| 4* | 30.42 | 76 | 0.400 | 46.8 | 48 | 0.975 | 1.375 | 6/4 |
| 5#* | 36.6 | 84 | 0.435 | 337.5 | 70 | 4.821 | 5.256 | 2/4 |
| | | | | | | | | 1/3 |
| 6 | n.d. | n.d. | n.d. | n.d. | n.d. | n.d. | n.d. | 0/1 |

n.d.-no death

TABLE 11
Level of IL-1, TNF, soluble receptors: IL-IRA and sTNF during
the treatment by Serum N1 of the experimental Dengue (type 2)
virus infection (Group 5)

| | IL-1 | | IL-1/ | TNF | | TNF/ | | Surviv- |
|---|---|---|---|---|---|---|---|---|
| | (pg/ | IL-IRA | IL-IRA | (pg/ | sTNFrI | sTNF | | al/ |
| Days | ml) | (pg/ml) | (I) | ml) | (pg/ml) | (II) | I + II | dead |
| 0 | 2.95 | 30 | 0.083 | 1.17 | 17 | 0.068 | 0.151 | 10/0 |
| 1 | 10.6 | 70 | 0.151 | 8.19 | 32 | 0.256 | 0.407 | 10/0 |
| 2 | 16.8 | 65 | 0.258 | 26.9 | 37 | 0.727 | 0.985 | 10/0 |

beginning of the treatment

| | | | | | | | | |
|---|---|---|---|---|---|---|---|---|
| 3 | 22.4 | 90 | 0.248 | 28.4 | 66 | 0.430 | 0.678 | 10/0 |
| 4 | 28.6 | 90 | 0.317 | 32.6 | 74 | 0.440 | 0.757 | 10/0 |
| 5 | 38.8 | 96 | 0.404 | 196.8 | 89 | 2.21 | 2.614 | 10/0 |
| 6 | 52.4 | 98 | 0.534 | 326.6 | 98 | 3.33 | 3.866 | 2/8 |
| 7 | n.d. | n.d. | n.d. | n.d. | n.d. | n.d. | n.d. | 0/2 |

m.t.d.-mean time to death—6.21 days
n.d.-no death

TABLE 12
Level of IL-1, TNF, soluble receptors: IL-IRA and sTNF during the
treatment by serum N2 of the experimental Dengue (type 2) virus
infection (Group 6)

| | IL-I | | IL-I/ | TNF | | TNF/ | | Surviv- |
|---|---|---|---|---|---|---|---|---|
| | (pg/ | IL-IRA | IL-IRA | (pg/ | sTNFrI | sTNF | | al/ |
| Days | ml) | (pg/ml) | (I) | ml) | (pg/ml) | (II) | I + II | dead |
| 0 | 2.95 | 30 | 0.083 | 1.17 | 17 | 0.068 | 0.151 | 10/0 |
| 1 | 10.6 | 70 | 0.151 | 8.19 | 32 | 0.256 | 0.407 | 10/0 |
| 2 | 16.8 | 65 | 0.258 | 26.9 | 37 | 0.727 | 0.985 | 10/0 |

the beginning of the treatment

| | | | | | | | | |
|---|---|---|---|---|---|---|---|---|
| 3 | 28.4 | 75 | 0.378 | 30.6 | 50 | 0.612 | 0.990 | 10/0 |
| 4 | 35.2 | 84 | 0.419 | 48.8 | 54 | 0.903 | 1.322 | 8/2 |
| 5 | 42.4 | 88 | 0.481 | 316.4 | 76 | 4.16 | 4.541 | 2/6 |
| 6 | n.d. | n.d. | n.d. | n.d. | n.d. | n.d. | n.d. | 0/2 |

n.d.-no death

TABLE 13
Effects of the different methods of treatment of the experimental Dengue
(type 2) virus infection

| | Scheme of | | |
|---|---|---|---|
| Group | Treatment | Survival/dead | m.t.d. |
| 2 | Virus control | 0/10 | 4.76 |
| | | 2 mice - on 4 day | |
| | | 8 mice - on 5 day | |
| 3 | doxycycline (from | 0/10 | 6.00 |
| | the 2 day till 5 day | 10 mice on day 6 | |
| | after infection) | | |
| 4 | doxycycline (from | 0/10 | 4.62 |
| | the 3 day till 5 day | 4 mice - on 4 day | |
| | after infection) | 5 mice - on 5 day | |
| | | 1 mice - on 6 day | |
| 5 | serum 1 (from the | 0/10 | 6.21 |
| | 2 day till 5 day | 8 mice - on 6 day | |
| | after infection) | 2 mice - on 7 day | |
| 6 | serum 2 (from the | 0/10 | 4.92 |
| | 2 day till five day | 2 mice - on 4 day | |
| | after infection) | 6 mice - on 5 day | |
| | | 2 mice - on 6 day | |

m.t.d. - mean time to death - 4.92 days

### EXAMPLE 3

### Treatment of Endotoxic Shock, Mousepox, Lassa Fever, Hemorrhagic Fever with Renal Syndrome (HFRS) and Dengue Fever with a Tetracycline Compound, IL-1Ra and Combinations Thereof

a. Expression of Soluble IL-1 Receptor Antagonist (IL-1Ra) in E. coli

The coding region of the IL-1Ra (residues 3-152, numbering according to Eisenberg et al. (1990) Nature 343:341-346; see, also Arend et al. (1990) J.

Clin. Invest. 85:1694-1797 and Hannum et al. (1990) Nature 343:336-340) as amplified from U937 cDNA by PCR with the introduction of an additional glycine residue, a BamHI restriction site at the 5′ end and an EcoRi site at the 3′ end (5′ oligonucleotide CGG GAT CCG GGA GAA AAT CCA GCA AGA TG SEQ ID NO. 25; 3′ oligonucleotide CGG AAT TCC CCT ACT CGT CCT GGA SEQ ID NO. 26). Using these primers, the mature recombinant IL-1Ra protein has the N-terminal sequence GSGRK, which is different from that of the native IL-1Ra protein, which is RPSGRK. The PCR product was introduced into the fusion protein expression vector pGEX-2T (Pharmacia; see, also Smith et al. (1988) Gene 67:21-40) and transformed into the E. coli strain NM554 (well known, see, e.g., Raleigh et al. (1988) Nucl. Acids Res. 16:1563-1575; and commercially available from, for example, Stratagene, La Jolla, Calif.). The expressed fusion protein glutathione S-transferase (GST)-IL-1Ra is cleaved with thrombin to obtain an authentic recombinant IL-1Ra protein.

b. Monitoring Production of TNF, Soluble TNF Receptor (sTNF R), IL-1, IL-1Ra in the Following Disease Models

There are disease models for monitoring disease progression and the efficacy of various treatment protocols. Exemplary models are as follows.

(1) Schwarzmann Reaction (Endotoxic Shock)

Endotoxic shock is accompanied by an increased IFN, TNF and IL-1 production, which simulates bacterial infection. BALB/c mice model are used in this study.

(2) Ectomelia (Mousepox)

BALB/c mice model are used in this study. Development of this lethal disease is accompanied by the increased TNF, IL-1 and IFN production.

Ectomelia virus gains entry through minute abrasions of the skin where it multiplies to produce a primary lesion. While this lesion is developing, a series of invasive steps produce a secondary viremia that seeds the skin and other organs with virus. A rash appears about 3 days after the primary lesion occurs.

(3) Experimental Lassa Fever

CBA/calac mice, which are highly sensitive to Lassa virus infection, are used in this study. Infection with the Lassa virus in the CBA/calac mice is accompanied by inflammation characterized histologically by cerebral edema, functional activity of kupffer cells, and necrosis of individual hepatocytes. Marked cytokine production also accompanies the disease development.

(4) Experimental HFRS Fever (Hantaan Virus)

C57B1/6 mice, which are highly sensitive to Hantaan virus infection, are used in this study. Development of this lethal disease is accompanied by the increased TNF and IL-1 production.

(5) Experimental Dengue Fever

BALB/c mice are used in this study. The mice are infected with denver fever virus. Development of this lethal disease is accompanied with by increased TNF, IL-1 and IFN production.

The data on dynamics of TNF, IL-1, sTNF and IL-1ra production and also dynamics of viremia are collected. These data allow the interrelationships between these cytokines, soluble receptors and the disease course to be determined. The scheme of administration of the soluble IL-1raand anti-TNF and anti-IL-1 drugs, which are likely to provide the healing of Systemic Inflammatory Response Syndrome (SIRS) in the above models, are based on the results thus obtained.

### EXAMPLE 4

### Treatment of the Dengue Virus Infection with Various Tetracycline and Tetracycline-like Compounds

Materials

Virus

Dengue virus type 2. Virus amplification by two passes through the brains of suckling mice. Mice were infected with 5 LD₅₀'s of virus.

Animals

160 mice BALB/c (haplotype H-2d), age 4 weeks were used for the experiment.

Experimental Scheme

A groups, control groups (virus only; 50 mice) Group A1, 20 mice, was the control group for mortality.

Group A2, 30 mice, was used for obtaining blood samples on the day (0) and days 1, 3, 5 and 6 post infection. Blood samples were obtained from the orbital sinuses (at every time point 3 mice were used for harvesting blood). All blood samples (500 μl) were frozen (−70 C.). After completion of the experiment, the concentrations of TNF and IL-1 were measured.

B groups, 60 mice, treatment with tetracycline hydrochloride (20 mg/kg) from the third day before the virus infection until 8 days after virus injection administered twice per day, orally in a volume of 30 μl.

Group B1, 20 mice, control for mortality.

Group B2, 40 mice, was used to obtain blood samples on the day (−1), (0) and days 1, 3, 5, 6, 7 and 12 post infection. Blood samples were obtained from the orbital sinuses (at every time point 3 mice were used for harvesting blood). All blood samples (500 μl) were frozen (−70 C.). After completion of the experiment the concentrations of TNF, IL-1 were measured.

C groups, 60 mice, treatment with Vybromycine suspension (20 mg/kg) from the third day before the virus infection until 8 days after virus injection, twice per day, orally in a volume of 30 μl.

Group C1, 20 mice, control for mortality.

Group C2, 40 mice, was used to obtain blood samples on day (−1), (0) and days 1, 3, 5, 6, 7, 8 and 12 post infection. Blood samples were obtained from the orbital sinuses (on every time point 3 mice were used for harvesting blood). All blood samples (500 μl) were frozen (−70 C.). After the whole experiment had finished, the concentrations of TNF, IL-1 were measured.

D groups, 60 mice, treatment with Terramycine (20 mg/kg) from the third day before the virus infection until 8 days after virus injection, twice per day, intramuscularly in volume 100 μl.

Group D1, 20 mice, control for mortality.

Group D2, 40 mice, was used to obtain blood samples on day (−1), (0) and days 1, 3, 5, 6, 7, 8 and 12 post infection. Blood samples were obtained from the orbital sinuses (on every time point 3 mice were used for harvesting blood). All blood samples (500 μl) were frozen (−70 C.). After the whole experiment had finished, the concentrations of TNF, IL-1 were measured. On the third day after challenge by the Dengue virus all samples taken from the infected mice were tested by RT-PCR for the virus detection.

TABLE 13
Dynamics of the changes of the concentrations of TNF-α and
IL-1β in the serum of animals from all Groups.

| | | | | | |
|---|---|---|---|---|---|
| | | IL-1 | TNF | | |
| Group | Days | pg/ml | pg/ml | Survival/total | amount |

Groups A

| | | | | | Group A1 (20 |
|---|---|---|---|---|---|
| | | | | | mice) % (survival) |
| A2 (virus control (30 mice) | | | | | 5 |
| | 0 | 7.0 | 18.4 | 20/20 | m.t.d. = 5.5 |
| | 1 | 12.2 | 22.6 | 20/20 | |
| | 3 | 54.8 | 50.8 | 20/20 | |
| | 5 | 80.2 | 112.5 | 12/20 | |
| | 6 | 166.8 | 136.6 | 4/20 | |
| | 7 | n.d | n.d | 1/20 | |
| | 12 | | | 1/20 | |
| | | | | | Group B1 (20 |
| | | | | | mice) % (survival) |
| B2 (tetracycline treatment; 40 mice) | | | | | 40 |
| | −1 | 6.8 | 18.4 | 20/20 | m.t.d. = 5.84 |
| | 0 | 6.8 | 16.0 | 20/20 | |
| | 1 | 10.8 | 16.6 | 20/20 | |
| | 3 | 46.8 | 14.0 | 20/20 | |
| | 5 | 66.0 | 28.8 | 16/20 | |
| | 6 | 56.8 | 38.4 | 11/20 | |
| | 7 | 10.2 | 33 | 8/20 | |
| | 12 | 7.4 | 19.6 | 8/20 | |
| | | | | | Group C1 (20 |
| | | | | | mice)% (survival) |
| C2 (Vybromycine treatment; 40 mice) | | | | | 20 |
| | −1 | 7.0 | 20.4 | 20/20 | m.t.d. = 6.7 |
| | 0 | 7.0 | 18.8 | 20/20 | |
| | 1 | 11.6 | 12.6 | 20/20 | |
| | 3 | 60.0 | 10.8 | 20/20 | |
| | 5 | 62.0 | 16.0 | 19/20 | |
| | 6 | 84.4 | 34.0 | 15/20 | |
| | 7 | 64.0 | 30.6 | 5/20 | |
| | 8 | 30.0 | 26.0 | 4/20 | |
| | 12 | 17.8 | 22.2 | 4/20 | |
| | | | | | Group D1 (20 |
| | | | | | mice)% (survival) |
| D2 (Terramycine treatment; 40 mice) | | | | | 15 |
| | −1 | 7.2 | 18.8 | 20/20 | m.t.d. = 6.53 |
| | 0 | 7.0 | 17.0 | 20/20 | |
| | 1 | 21.8 | 15.2 | 20/20 | |
| | 3 | 112.0 | 25.6 | 20/20 | |
| | 5 | 84.0 | 26.0 | 19/20 | |
| | 6 | 80.0 | 36.2 | 11/20 | |
| | 7 | 76.0 | 28.0 | 6/20 | |
| | 8 | 42.0 | 20.0 | 3/20 | |
| | 12 | 16.0 | 18.0 | 3/20 | |

The results set forth in Table 13 show that in the virus control group A2, the concetration of IL-1 increased 24-fold during the course of the disease (from the day 0 until the day 7), and the concentration of TNF increased 7-fold; m.t.d. in this group was 5.5 days and all animals died. In group B2, which was treated with tetracycline therapy, 40% of the animals survived (the m.t.d. of 5.84 is not statistically different from group A2). The concentration of IL-1 increased 10-fold by day 5 of the disease, the concentration of TNF increased 2-fold. the level of the cytokines in the serum of the animals of this group was statistically lower than in the control A2 group. In group C2, which was treated vibromycline, 20% of the animals survived, m.t.d. was 6.7 statistically higher than in the control A2 group. The concentration of IL-1 increased 12-fold by day 6 of the infection, and the concentration of TNF increased 3-fold. The level of cytokines in the serum of the animals of this group was statistically lower than in the control A2 group. In group D2, which was treated with terramycine, 15% of the animals survived, m.t.d. was 6.53, which is statistically longer in the control A2 group. The concentration of IL-1 increased 16-fold by day 3 of the disease and stayed at this level until the day 7. The concentration of TNF increased 2-fold by day 6 of the disease. The levels of the cytokines in the serum of the animals in this group were statistically lower than in the control group A2. Soluble tetracycline was most effective.

### EXAMPLE 5

### Treatment of the Dengue Virus Infection with Various Tetracyclines and serum

Virus

Dengue virus, type 2. All work with infectious virus was performed in the maximum-containment biosafety level-3 (BSL-3) of the SRC VB))Vector)). This virus was amplified in the brain of the suckling mice and was collected to produce stocks. This stock virus suspension was stored at −40 C., contained 6.8 LD₅₀/ml (in the mice BALB/c by intraperitoneal challenge). For infecting mice we used 5 LD₅₀ virus.

Animals

4-week-old BALB/c mice (haplotype H-2d), which weighed 12-14 grams, were used in the experiments with Dengue virus. The animals were received from the vivarium of SRC VB ((Vector)) and kept on a standard ration.

RT-PCR Procedure

Primers for Dengue virus type 2 detection were: Upper 5′AATATGCTGAAACGCGAGAGAAACCG (position 136-161) SEQ Lower 5′AAGGAACGCCACCAAGGCCATG (position 237-258) SEQ ID No. 24.

RNA was extracted from the serum of the infected animals (mice) by means of RNeasy Kits (Quiagen, Germany). For RT-PCR Titan-Kits (Berhringer, Germany) were used. Reverse transcription was conducted at 42° C. for 60 min, followed by 40 amplification cycles at 94° C. for 30 sec, at 55° C. for 1 min, and at 68° C. for 2 min, with a final extension at 68° C. for 7 min. Amplification was conducted in 0.2-ml tubes with a model BIS-105M thermocycler (Russia). The virus detection was provided by PCR on the second day after animals infection.

Experimental Scheme

Mice of all groups were infected by 5 LD₅₀ of Dengue virus.

Groups A—control groups (only virus).

Group A1—20 mice—control for mortality.

Group A2—30 mice—was used for obtaining blood samples on day (0) and on days 1, 3, 5 and 6 post infection. The blood samples were obtained from the orbital sinuses (at every time point 3 mice were used for harvesting blood). All blood samples (500 μl each) were frozen at −70° C. After completion of the experiment, the concentrations of TNF-α and IL-1β were measured. Groups C, 36 mice, were the Human serum treatment group. Treatment was carried out with the Human serum stimulated by Vibromycine. The Human serum was obtained from the blood of a human administered vibromycine (150 mg) orally twice a day (every 12 hours). The human blood was taken on the second and the third day after the beginning of the stimulation. The concentration in the human serum of IL-1RA was 184 pg/ml, and the concentration of sTNFrI was 950 pg/ml.

Treatment of the mice commenced on the third day after viral infecting of the mice and continued until day 8. It was administered intraperitonealy twice a day in the volume of 200 μl per dose. The dose of the infusing human serum is about 16% of the blood volume of a mouse.

Groups B—Tetracycline treatment groups.

Treatment with Tetracycline hydrochloride (100 μg in a volume of 30 μl) was carried out from the third day after virus infection until day 8, twice per day, orally. Tetracycline is more soluble than vibromycine so that is could be administered more readily in solution to the mice.

Group B1—control for mortality (20 mice).

Group B2—30 mice—was used for obtaining blood samples on day (0) and days 1, 3, 5, 6 and 12 post infection. Blood samples were obtained from the orbital sinuses (at every time point 3 mice were used for harvesting blood). All blood samples (500 μl each) were frozen −70° C. After completion of the experiment, the concentrations of TNF-α, and IL-1β were measured.

Groups C

Group C1—control for mortality. 10 mice.

Group C2—26 mice—was used for obtaining blood samples on day (0) and days 1, 3, 5 and 12 post infection. Blood samples were obtained from the orbital sinuses (at every time point 3 mice were used for harvesting blood). All blood samples (500 μl) were frozen and −70° C. After completion of the experiment, the concentrations of TNF-α and IL-1β were measured.

Groups D Control for human serum treatment groups.

The control for treatment was human serum obtained from the human before the Vibromycine stimulation. This “normal” human serum contained 24.4 pg/ml of IL-1RA and 25.0 pg/ml of sTNFR1. The volume dose and method of infusion were the same as during the Human serum treatment course. Treatment with the normal human serum commenced on the third day after virus infection until day 7, twice per day, intraperitonealy in a volume of 200 μl per dose. The dose of the infusing normal human serum was about 16% of the blood volume of a mouse.

Group D1—10 mice—control for mortality.

Group D2—26 mice—was used for obtaining blood samples on day (0) and days 1, 3, 5 and 6 post infection. Blood samples were obtained from the orbital sinuses (at every time point 3 mice were used for harvesting blood). All blood samples (500 μl) were frozen at −70° C. After completion of the experiment, the concentrations of TNF-α and IL-1β were measured.

Groups E. Treatment with anti-TNFα serum.

Group E1—10 mice.

For treatment rabbit serum prepared against the human TNF-α was used. The neutralizing activity of this rabbit's serum was 1 ng/ml. Treatment with anti-TNF-α serum commenced on the third day after virus infection until day 7, twice per day, intraperitonealy in a volume of 200 μl per dose. The dose of the infusing anti-TNF-α serum represented 16% of the blood volume of a mouse.

Group E2-10 mice.

The treatment with the normal rabbit serum was carried out from the third day after virus infection until day 6, twice per day, intraperitonealy in a volume of 200 μl per dose. The dose of the infusing normal rabbit serum represented 16% of the blood volume of a mouse.

Results

The results of the experiments show that the oral administration of Tetracycline (groups B) for the treatment of the experimental Dengue fever in mice (20 mg/kg, daily) prolongs (statistically significant) the lifetime of the animals, and increases (statistically significant) the number of the surviving mice (Table 14). The data (see Table below) shows that treatment considerably reduces inflammatory cytokines such as TNFα and IL-1β (Table 15). Treatment with stimulated human serum (groups C) containing the increased concentrations of the receptors of the cytokines also prolonged the lifetime of the mice, and increased the number of surviving animals. The results of the treatment by the normal human serum (groups D) did not reveal any significant differences from the results in the Control group A. Hence this data demonstrates the essential role of TNFα in the development of the experimental Dengue fever.

These results are further confirmed by the results of the anti-TNFα serum treatment (group E1). In this group 60% of all animals survived and the lifetime was significantly higher.

TABLE 14
The average lifetime and the data of the mortality among the treated
mice with the experimental Dengue fever

| Group | Scheme of Treatment | Survived/died | m.t.d. |
|---|---|---|---|
| A1 | virus control | 0/20 | 6.94 + 0.02 |
| B1 | Tetracycline treatment | 9/11 | 8.40 ± 0.73* |
| C1 | Human serum (with | 3/7 | 8.54 ± 0.42* |
| | sTNF RI and IL-1RA) | | |
| | treatment | | |
| D1 | <Normal>human serum | 0/10 | 7.00 ± 0.31 |
| E1 | Anti-TNF-α serum | 4/6 | 8.70 ± 0.48*,** |
| | treatment | | |
| E2 | Normal rabbit serum | 0/10 | 6.94 ± 0.02 |
| | treatment | | |

*the difference with the group A is statistically significant (P < 0.1)
**the difference with the group E2 is statistically significant (P < 0.1)

TABLE 15
Dynamics of the changes of the concentrations of TNF-α and IL-β in
the serum of the animals with the experimental Dengue fever

| | | | IL-1 | TNF |
|---|---|---|---|---|
| Group | Scheme of Treatment | Day | pg/ml | pg/ml |
| A2 | virus control | 0 | 6.2 | 8.0 |
| | | 1 | 12.1 | 14.4 |
| | | 3 | 32.8 | 36.8 |
| | | 5 | 62.6 | 116.4 |
| | | 6 | 88.4 | 459.2 |
| B2 | Tetracycline treatment | 0 | 6.0 | 7.8 |
| | | 1 | 12.0 | 13.8 |
| | | 3 | 36.0 | 38.2 |
| | | 5 | 48.6 | 56.2 |
| | | 6 | 62.4 | 156.8 |
| | | 12 | 15.6 | 18.0 |
| | | 21 | 5.8 | 7.4 |
| C2 | Human serum (with | 0 | 6.2 | 7.8 |
| | STNFrI IL-1RA) treatment | 1 | 12.2 | 14.0 |
| | | 3 | 36.8 | 35.8 |
| | | 5 | 52.4 | 78.2 |
| | | 12 | 18.2 | 19.2 |
| | | 21 | 6.6 | 7.6 |
| D2 | <Normal> human serum | 0 | 7.0 | 7.6 |
| | treatment | 1 | 12.2 | 13.6 |
| | | 3 | 36.4 | 36.8 |
| | | 5 | 60.8 | 98.2 |
| | | 6 | 84.2 | 320.0 |

### EXAMPLE 6

### Treatment of Marburg Virus Infection

Virus

Marburg virus strain Popp was received from the Belarussian Institute of Epidemiology and Microbiology. This virus was amplified in Vero E6 cells and the supernatant was collected to produce stocks. This stock virus suspension has been stored at −70° C., contained 10⁷7 PFU/ml. All work with infectious virus was performed in the maximum-containment biosafety level—4 (BSL-4) of the SRC VB (Vector).

Animals

Outbred guinea pigs (male) 200-220 grams were used in the experiments with Marburg virus.

Experimental Scheme

All animals were divided into groups, each contained 6 animals.

The guinea pigs were infected by the 5 LD₅₀ of the Marburg virus. Animals of the group A were used only for the virus control. Animals of the group B after infection were treated by the human serum (SERUM1) with IgG against Marburg (titer IgG in ELISA 1:80), without IgG against Ebola and sTNFrI (950 pg/ml), TNFα (7.8 pg/ml), IL-1RA (136 pg/ml), IL-1β, ((3 pg/ml), Animals of the group B were given SERUM1 intracardially from day 3 after virus infection until day 14, every day at the following doses:

3 day—200 μl

4 day—200 μl

5 day—400 μl

6 day—400 μl

7 day—600 μl

8 day—600 μl

9 day—600 μl

10 day—800 μl

11 day—800 μl

12 day—800 μl

13 day—800 μl

14 day—800 μl

Animals of the group C were treated by the human serum with IgG against Marburg virus (titer IgG in ELISA 1:80), without IgG against Ebola, the concentration of TNFα-7.8 pg/ml, sTNFrI-21 pg/ml, IL-1β-3 pg/ml, IL-IRA-24.4 pg/ml Serum 2.

Animals of the group C were given Serum 2 intracardially from day 3 after virus infecting until day 12, every day, at the following doses:

3 day—200 μl

4 day—200 μl

5 day—400 μl

6 day—400 μl

7 day—600 μl

8 day—600 μl

9 day—600 μl

10 day—800 μl

11 day—800 μl

12 day—800 μl

Animals of the group D were treated with the human serum without antibodies against Marburg virus and without antibodies against Ebola virus, and with sTNFrI-880 pg/ml,TNFα-7.2 pg/ml, IL-1β-3 pg/ml, IL-1RA-146 pg/ml (Serum 3).

Animals of group D were given Serum 3 intracardially from 3 day after virus infecting until 12 day, every day, at the following doses:

3 day—200 μl

4 day—200 μl

5 day—400 μl

6 day—40 μl

7 day—600 μl

8 day—600 μl

9 day—600 μl

10 day—800 μl

11 day—800 μl

12 day—800 μl

Animals of the group E were treated with human serum without the antibodies against Marburg and Ebola viruses, and the concentrations of TNF α-7.0 pg/ml, sTNFrl-20pg/ml, IL-1β-3 pg/ml, IL-1RA-20 pg/ml (SERUM 4). Animals of the group E were given Serum 4 intracardially from 3 days after virus, injecting every day, until 12 day, at the following doses:

3 day—200 μl

4 day—200 μl

5 day—400 μl

6 day—400 μl

7 day—600 μl

8 day—600 μl

9 day—600 μl

10 day—800 μl

11 day—800 μl

12 day—800 μl

On the third day after the challenge by the Marburg virus the blood samples taken from all infected guinea pigs (groups A, B, C, D, E) were tested by RT-PCR. This RT-PCR test was performed for the confirmation of the virus infection and showed positive amplification using a cDNA segment of Marburg virus with the approximate size about 420 bp. Detection of the virus by the PCR method in the blood samples performed before the challenge (0 day) showed no Marburg virus.

On day 7 a positive result by RT-PCR test was obtained. On the 27th day after the challenge, no Marburg virus was detected in the blood samples of the surviving animals.

TABLE 16
Mortality, average lifetime among the infected by the Marburg virus
guinea pigs

| | Serum | Survived/total | | |
|---|---|---|---|---|
| Group | treatment | amount | % of survival | M.T.D. |
| A | control: | 0/6 | 0% | 11.49 + 0.64 |
| | only virus | | | |
| B | Serum 1 | 4/6 | 66% | 13.51 + 0.80* |
| C | Serum 2 | 0/6 | 0% | 11.90 + 0.48 |
| D | Serum 3 | 1/6 | 16% | 11.73 + 0.53 |
| E | Serum 4 | 0/6 | 0% | 11.62 + 0.48 |

*statistically significant (P(0.01)

Results

All guinea pigs in groups A, C and E died, and the average lifetime was not statistically different from the control group A. In the animals of the group B treated by with SERUM1, which contains antibodies against Marburg virus and soluble receptors sTNFR and IL-IRA, a tendency of increasing survival of animals was observed and the prolongation of lifetime was statistically significant. Human soluble receptors (sTNFR1 and IL-IRA) were detected in the blood samples of the treated guinea pigs on day (0) before infecting (as a control) and on day 7 after infecting with the Marburg virus, and on the 27th day among the survived guinea pigs. The detection was performed using ELISA-kits of R&D Production. The human soluble receptors sTNFR1 and IL-IRA were detected in the blood of the animals. Without being bound by any theory, it appears that these receptors were used for the neutralization of the inflammatory cytokines produced during the development of the Marburg fever in the animals. The serum of the surviving guinea pigs after Marburg infection was used for the detection of the specific IgG by ELISA and Western blot (groups of guinea pigs A, B, C) on days (0), 27 and 35. On day ((0)) no specific IgG was detected. But on day 27 and 35 the specific antibodies against Marburg virus were found at a titer of 1:80. At the same time no antibodies against Ebola virus were detected.

It appears from the combination of the low titer of the antibodies against the Marburg virus with sufficient concentrations of the soluble receptors of the inflammatory cytokines can influence the development and outcome of the experimental Marburg fever.

### EXAMPLE 7

### Treatment of E. Coli Infection

Bacterial Strain

Enterohemorrhagic Escherichia coli (EHEC), 0 157:H7 strain, serotype 105282 was used these experiments. The organisms were incubated in LB medium for 24 h ar 37 C. After one passage viable counts were determined by plating on the agar media. Titer of E. coli was 10⁸ PFU. E. coli suspension was prepared by washing the bacterial pellet twice in the phosphate-buffered saline (PBS; pH 7.4).

Dosage and Method of Infecting

The bacterial suspension (10⁷ PFU) in the volume of 30 μl was infused to the mice intragastrically through the soft polyethylene catheter.

Mice. 4-week-old male BALB/c mice (halpotype H-2d) were used in the experiments. The blood volume per mouse was approximately 1.2 ml. All animals were divided into the following groups.

Groups A. Control groups. All animals were infected by E. coli suspension.

Group A-1, 10 mice, control for mortality. Group A2, 20 mice, was used to obtain blood samples on day “0” and day 1, 2, 3, 5 post-infection. Blood samples were obtained from the orbital sinuses (on every time point 3 mice were used for harvesting blood). All blood samples (500 μl each) were frozen −70 C. After the whole experiment had finished, the concentrations of TNF, IL-1 were measured.

Groups B. Treatment groups (B1 and B2).

Treatment was carried out with the Human serum containing IL-RA and sTNFrI. The Human serum was obtained from the blood of human taking orally Vibromycine in dose of 150 mg twice per day (every 12 hours). The Human blood was taken on the second day and the third day after the beginning of taking antibiotic. The concentration in the Human serum of IL-1RA was 184 pg/ml, and the concentration of sTNFrl was 950 pg/ml. The treatment was started from the second day after bacterial infecting of the mice and continued until 9 day, twice per day, intraperitoneally, in the volume of 200 μl per dose. The dose of the transfusing Human serum presented 16% of the blood volume of a mouse.

Group B1, 10 mice, control for mortality.

Group B2, 26 mice, was used from obtaining blood samples on day “0” and day 1, 2, 3, 5, 12, 21 post infection. Blood samples were obtained from the orbital sinuses (on every time point 3 mice were used for harvesting blood). All blood samples (500 μl each) were frozen −70° C. After the whole experiment had finished, the concentrations of TNF, IL-1 were measured.

Groups C. Control for Treatment groups.

Treatment was carried out with the ((Normal)) Human serum. The concentration in the ((Normal)) Human serum of IL-1RA was 24.4 pg/ml, and the concentration of sTNFrI was 22 pg/ml. The concentration of IL-1β pg/ml, the concentration of TNFα-7.6 pg/ml. The treatment was started from the second day after bacterial infecting of the mice and continued until 7 day, twice per day, intraperitonealy, in the volume of 200 μl per dose. The does of the transfusing Normal Human serum presented 16% of the blood volume of a mouse. All animals died on day 7 after bacterial infection.

Group C1, 10 mice, control mortality.

Group C2, 26 mice, was used to obtain blood samples on day “0” and day 1, 2, 3, 5, 6 post infection. Samples were obtaining from the orbital sinuses (on every time point 3 mice were used for harvesting blood). All blood samples (500 μl each) were frozen −70 C. After the whole experiment had finished, the concentrations of TFNa, IL-1β were measured.

Results

The results of the experiments show that infecting the mice with a pathogenic strain of E. coli leads to the death of all mice. The clinical manifestations of the experimental disease caused by this strain of E. coli have many common features with the experimental fevers in animals such as Dengue, Lassa, and Machupo. The presence of sepsis in the infected animals was confirmed by demonstrating E. coli in the blood of the animals on the 6^{th} day after infecting while it was not present before infecting. All infected mice showed intensified production of TNFα and IL-1β. Infusion of normal nonstimulated human serum had no effect on the levels of inflammatory cytokines nor did it prolong the lifetime of the animals or the number of survivors. Treatment with vibromycine stimulated human serum that contained resulting higher concentrations of sTNFrI and IL-1RA provides a statistically significant prolongation of lifetime of the infected mice, the survival of 4 of 10 mice and a decrease in production of the cytokines as sTNFrI and IL-1RA.

TABLE 17
The effects of the treatment of the experimental bacterial shock

| Group | Scheme of treatment | Survived/died | m.t.d. |
|---|---|---|---|
| A | E. coli control | 0/10 | 5.84 ± 0.19 |
| B | Human serum with sTNF and | 4/6 | 7.14 ± 0.49* |
| | I1-1RA, stimulated. | | |
| C | Human serum (normal) | 0/10 | 6.36 ± 0.29 |

*the difference from group A is statistically significant (P(0.05)

TABLE 18
Dynamics of the changes of concentrations of TNF-α and IL-1β in
the serum of animals with experimental bacterial shock.

| | | IL-1 | TNF |
|---|---|---|---|
| Group | Days | pg/ml | pg/ml |
| A2 | 0 | 7.8 | 5.4 |
| | 1 | 15.0 | 8.0 |
| | 2 | 23.0 | 10.0 |
| | 3 | 40.0 | 16.0 |
| | 5 | 190.0 | 362.0 |
| B2 | 0 | 7.2 | 5.6 |
| | 1 | 17.0 | 9.0 |
| | 2 | 24.0 | 11.0 |
| | 3 | 33.0 | 14.0 |
| | 5 | 86.0 | 136.0 |
| | 12 | 11.0 | 10.6 |
| | 21 | 6.2 | 5.0 |
| C2 | 0 | 7.2 | 5.4 |
| | 1 | 15.0 | 8.0 |
| | 2 | 24.0 | 11.0 |
| | 3 | 40.0 | 17.0 |
| | 5 | 172.0 | 316.0 |
| | 6 | 236.0 | 488.0 |

### EXAMPLE 8

### In Nitro Activation of Mononuclear Human Cells by Antibiotics

1. Cells

Mononuclear cells were obtained from human blood, which had been collected in tubes with Heparin (5 ED heparin/ml) and centrifuged on Hustopaqe (p=1.077), 1000×g, 30 minutes. Mononuclear cells were washed twice with RPMI-1640 medium (pH 7.2). The concentration of the cells was 5×10⁶/ml.

2. Activation of Cells

One portion of the cells was used as control, without any activation (in a volume 2 ml). A second portion was used for the tetracycline activation at a concentration of 0.06 mg/ml (in a volume of 2 ml). The third portion was used for the terramycine activation at a concentration of 0.06 mg/ml (in a volume of 2 ml). The activation continued for 2 hours, and the cells then were washed twice with the medium RPMI-1640 (pH 7.2). A monolayer was formed (2×10⁶/ml) and the cells were cultured at 37° C., 95% humidity, atmosphere of 5% of CO₂. Samples of activated mononuclear cells were taken on the third, 6th and 24th hours after the beginning of the contact. The concentrations of sTNFrI and IL-1RA were measured using standard ELISA-kits by R&D Systems.

The results of the experiment showed that the production of the receptors such as sTNFrI and IL-1RA are induced in vitro using Tetracycline and Terramycine. The production of the receptors by the activated cells was statistically significantly higher than the production by the non-stimulated cells. The concentrations of the receptors obtained in vitro are comparable to the concentrations obtained in vivo and even higher. For example, after vibromycine stimulation, the concentration of receptors in the donor serum (2 persons, on the 24th hour) were IL-1RA 126.8±6.8 pg/ml, sTNFrl 970±28.6 pg/ml (before the stimulation: IL-1RA 20±2.2 pg/ml and sTNFrl 22±3.4 pg/ml). After the tetracycline stimulation the concentrations of the same receptors in the donor serum (2person, at the 24th hour) was 130±6.8 pg/ml and 580±18.2 pg/ml.

TABLE 20
Dynamics of the concentrations of IL-1RA and sTNFrl

| | | IL-1RA | sTNFrl |
|---|---|---|---|
| Cells | Hours | pg/ml | pg/ml |
| only Cells | 0 | 27 + 1.4 | 18 + 1.6 |
| | 3 | 40 + 3.2 | 68 + 4.8 |
| | 6 | 58 + 4.6 | 44 + 3.2 |
| | 24 | 44 + 3.4 | 22 + 2.1 |
| Cells + Terramycine | 0 | 28 + 1.6 | 18 + 1.4 |
| | 3 | 93 + 6.2 | 313 + 10.4 |
| | 6 | 220 + 9.4 | 224 + 9.2 |
| | 24 | 185 + 8.6 | 264 + 9.6 |
| Cells + Tetracycline | 0 | 22 + 1.4 | 19 + 1.2 |
| | 3 | 86 + 4.6 | 185 + 8.4 |
| | 6 | 186 + 8.2 | 204 + 9.2 |
| | 24 | 140 + 7.6 | 201 + 8.6 |

### EXAMPLE 9

### Treatment of Septic Shock with Plasma from Tetracycline-injected Mice

1. Preparation of Plasma from Tetracycline-injected Mice

Sixty 7-8 week old Balb/c mice (H²⁻⁴ haplotype) were injected intramuscularly with tetracycline (58 mg/kilo in 0.1 ml of sterile PBS). Plasma (citrated)was collected from these mice at 24 hour postinjection. One 0.2 ml sample of the plasma from these mouse was tested for the presence of IL-1R and TNFα-RI&II. the reminder of the plasma from each mouse was pooled. After removing a small sample from this pool for testing for the above mentioned cytokines, the reminder of the plasma pool was stored at −85° C. until needed.

Thirty 7-8 week old female Balb/c mice (H^{2-d}) were injected with 0.1 ml of sterile PBS and their plasma was drawn at 24 hour postinjection. A sample of plasma from each mouse was tested for IL-1R and TNFα-RI&II. The remainder of the plasma from this group of mice was pooled. A sample of the pooled plasma was tested for the cytokines as described above.

2. Treatment of the Mice with Septic Shock

Fifty 6-8 week old female Balb/c mice (Haplotype as above) received concurrent intraperitoneal injections of 25 μg of Staphylococcus enterotoxin B (SEB) and 20 mg of galactosamine for the induction of Septic Shock. The mice were divided into the following treatment groups:

1) ten mice remained untreated and served as negative controls;

2) ten mice received an intramuscular injection of tetracycline (58 mg/kilo) on the day of induction, and on days 1, 2, 3 and 4 postinduction. These mice also received twice daily injections of 0.3 ml of plasma from mice treated with tetracycline on the day of induction and on days 1, 2, 3 and 4 postinduction;

3) ten mice received 0.3 ml of plasma from tetracycline-injected mice twice daily on the day of induction and on days 1, 2, 3 and 4 postinduction;

4) ten mice received intramuscular injection of tetracycline and 0.3 ml of plasma from tetracycline-injected mice once daily on the day of induction and on days 1, 2, 3 and 4 postinduction; and

5) ten mice received 0.3 ml of plasma from PBS-injected mice twice daily on the day of induction and on days 1, 2, 3 and 4 postinduction.

Ten mice were not induced for septic shock and served as normal controls.

Mortality among all groups of animals was recorded four times daily for 4 days (96 hours) postinduction.

All mice without the induced septic shock survived 96 hours postinduction. None of the mice with the septic shock treated with control plasma, i.e., plasma prepared from PBS-infected mice, survived 36 hours postinduction. About 20% of the mice with septic shock that were treated with either tetracycline or tetracycline-stimulated plasma alone survived 96 hours postinduction. About 40% of the mice with septic shock that were treated with tetracycline and tetracycline-stimulated plasma survived 96 hours postinduction. Therefore, combination therapy of tetracycline and tetracycline-stimulated plasma boosts the survival rate of the mice with the SEB-induced septic shock.

### EXAMPLE 10

### Effects of Plasma from Tetracycline-injected on the Outcome of Septic Shock in Mice and Protocols for Testing of Treatment Hemorrhagic Fevers in a Rodent Model

Individuals infected with gram negative bacteria such as Escherichia coli and Salmonella typhi develop a characteristic syndrome that includes acidosis, fever, hypotension, lactate release into the tissues, disseminated intravascular coagulation (DIC) and renal, hepatic and lung injury. These infections and the resulting syndrome can induce a lethal condition called septic shock (SS). Numerous studies have established that this pathologic picture is attributable almost entirely to secretion of TNFα by endotoxin-stimulated macrophages.

Mouse DIC and SS Models

Balb/c mice sensitized by administration of D-galactosamine and injected intraperitoneally with Staphylococcus enterotoxin B (SEB) are a well-established model for human septic shock with accompanying disseminated intravascular coagulation. This process is driven by the release of TNFα and IL-1 by antigen-stimulated macrophages. In this mouse model, death usually occurs within 24 hr or antigen challenge.

Phase I

1. Sixty, 7-8 week old female Balb/c mice (H²⁻⁴ haplotype) are injected intramuscularly with tetracycline (58 mg/kilo in 0.1 ml of sterile PBS).

2. Plasma (citrated) is collected from these mice at 24 hr postinjection. One 0.2 ml sample of plasma from each mouse is set aside for testing for the presence of IL-1R and TNFα-RI&II, the reminder of the plasma from each mouse is pooled. After removing a small sample from this pool for testing for the cytokines of interest, such as IL-1 an TNFα, the reminder of the plasma pool is be stored at −85° C. until needed.

3. Thirty, 7-8 week old female Balb/c mice (H^{2-d}) are injected with 0.1 ml of sterile PBS and their plasma drawn at 24 hr postinjection. A sample of plasma from each mouse will be tested for IL-1R and TNFα-RI&II and the remainder of the plasma from this group of mice will be pooled. A sample of the pooled plasma will be tested for cytokines as above.

Phase II

1. Fifty, 6-8 week old female Balb/c mice (Haplotype as above) receive concurrent ip injections of 25 μg of SEB and 20 mg of galactosamine.

10 mice remain untreated and serve as negative controls

10 mice receive an im injection of tetracycline (58 mg/kilo) on the day of induction, and on days 1, 2, 3 and 4 postinduction. These mice also receive twice daily injections of 0.3 ml of plasma from mice treated with tetracycline on the day of induction and on days 1, 2, 3 and 4 postinduction.

10 mice receive 0.3 ml of plasma from tetracycline-injected mice twice daily on the day of induction and on days 1, 2, 3 and 4 postinduction.

10 mice receive im injection of tetracycline and 0.3 ml of serum from tetracycline injected mice once daily on the day of induction and on days 1, 2, 3 and 4 postinduction.

10 mice receive 0.3 ml of serum from PBS-injected mice twice daily on the day of induction and on days 1, 2, 3 and 4 postinduction.

2. Ten mice as described above are not treated for induction of Septic Shock and will serve as normal controls.

3. Mortality among all groups of animals is recorded four times daily.

Design of Experiment

Investigation of Treatment of Yellow Fever Infection

1. Virus—Yellow fever—strain “Dakkar”, the stock virus suspension after passage sulking mice.

2. Animals—BALB/c, male, 4 weeks age, 140 animals.

Steps

1. Preparation of serum from mice after by injections of Doxycycline, (70 mice for group)

2. For mice infection used 5 LD₅₀ of YFV.

group A—control for YFV without treatment—10 mice.

group B—treatment of YFV by Doxycycline from the third day after infection, every day.

group C—treatment of YFV by Doxycycline from the third day after infection every 12 h.

group D—treatment of YFV by serum (with IL-1RA and sTNF) from the third day after infection, every day.

group E—treatment of YFV by serum (with IL-1RA and sTNF) from the third day after infection every 12 h.

group F—control virus: for detection soluble receptors (sTNF, IL-1RA) and cytokines (TNF and IL-1) in blood after infection (days 1, 2, 3, 4, 5, 6)—20 mice.

Investigation of Treatment of Lassa Fever Infection

1. Virus—Lassa fever—strain “Josiah”, the stock virus suspension after passage suckling mice.

2. Animals—CBA/calac, male, 4 weeks age, 140 animals.

Steps

1. Preparation of serum from mice after by injections of Doxycycline. (80 mice for group)

2. For mice infection used 10 LD₅₀ of LFV.

group A—control for LFV without treatment—20 mice.

group B—treatment of LFV by serum (with IL-1RA and sTNF) from the third day after infection, every day (20 mice).

group D—treatment of LFV by serum (with IL-1RA and sTNF) from the third day after infection every 12 h (20 mice).

### EXAMPLE 11

### Assays for TNF and IL-1 Receptors

Assays for IL-1 Receptors

Numerous bioassays used to detect and quantitate IL-1Ra are known. An assay used herein to determine IL-1Ra in blood and blood-derived fractions that have been treated with tetracycline or tetracycline-like compounds is the Quantikine IL-1ra mmunoassay, which is solid phase ELISA designed to measure IL-1Ra in cell culture supernate, serum, and plasma. It contains E. coli-derived recombinant human IL-1Ra as well as antibodies raised against the recombinant factor. This immunoassay has been shown to accurately quantitate the recombinant human IL-1ra. Results obtained during natural human IL-1ra showed linear curves that were parallel to the standard curves obtained using the E.coli-expressed Quantikine kit standards. These results indicate that the Quantikine Immunoassay kit can be used to determine relative mass values for natural human IL-1ra.

Principle of the Assay

This assay employs the quantitative sandwich enzyme immunoassay technique. A monoclonal antibody specific for IL-1Ra has been pre-coated onto a microplate. Standards and samples are pipetted into the wells and any IL-1Ra present is bound by the immobilized antibody. After washing away any unbound substances, an enzyme-linked polyclonal antibody specific for IL-1Ra is added to the wells. Following a wash to remove any unbound antibody-enzyme reagent, a substrate solution is added to the wells and color develops in proportion to the amount of IL-1Ra bound in the initial step. The color development is stopped and the intensity of the color is measured.

Assays for TNFs

Bioassays for sTNFR If typically involves measurement of the inhibitory effect of the soluble receptor on the cytotoxic activity TNF-α on a susceptible cell line. The Quantikine human sTNF RI Immunoassay is a solid phase ELISA designed to measure sTNF RI in cell culture supernate, serum, plasma and urine. It contains E. coli-expressed, recombinant human sTNF RI, as well as antibodies raised against this polypeptide. The recombinant protein represents the non-glycosylated, N-terminal methionyl form of the naturally occurring human soluble Type I receptor for TNF with an apparent molecular weight of approximately 18.6 kDa. This immunoassay has been shown to accurately quantitate the recombinant sTNF RI. Results obtained on samples containing natural sTNF RI showed linear curves that were parallel to the standard curves obtained using the Quantikine kit standards. These results indicate that Quantikine Immunoassay kit can be used to determine relative mass values of natural sTNF RI. Since the measurement of human sTNF RI by this immunoassay is relatively insensitive to added TNF-α or TNF-62 , it is probable that this measurement corresponds to the total amount of the soluble receptor present in samples, i.e., the total amount of free receptor plus the total amount of receptor bound to TNF.

Principle of the Assay

This assay employs the quantitative sandwich enzyme immunoassay technique. A monoclonal antibody specific for sTNF RI has been pre-coated onto a microplate. Standards and samples are pipetted into the wells an any sTNF RI present is bound by the immobilized antibody. After washing away any unbound substances, an enzyme-linked polyclonal antibody specific for sTNF RI is added to the wells. Following a wash to remove any unbound antibody-enzyme reagent, a substrate solution is added to the wells and color develops in proportion to the amount of sTNF RI bound in the initial step. The color development is stopped and the intensity of the color is measured.

TABLE 21
Exemplary levels of IL-1, TNF, IL-1RA and sTNF RI in samples
from normal volunteers

| | | | TNF | | |
|---|---|---|---|---|---|
| Sample | IL-1 | | pg/ml | IL-IRA | sTNF RI |
| 1. subject 1 serum 10/22 | 3 | pg/ml | 7.8 | 241.6 | |
| 2. subject 1 serum 12/06 | <3 | pg/mL | 7.8 | 136.0 | 950 |
| 3. subject 1 serum 12/07 | <3 | pg/mL | 7.8 | 100.8 | 970 |
| 4. subject 1 serum 12/08 | <3 | pg/mL | 7.8 | 184.8 | 875 |
| 5. subject 1 plasma 12/01 | <3 | pg/mL | 7.8 | 140.8 | 575 |
| 6. subject 1 plasma 12/03 | <3 | pg/mL | 7.8 | 82.4 | 825 |
| 7. subject 1 plasma 12/07 | <3 | pg/mL | 7.8 | 140.8 | 600 |
| 8. subject 2 serum 12/06 | 3 | pg/mL | 8.6 | 140.8 | 1650 |
| 9. subject 2 serum 12/07 | 3.9 | pg/mL | 8.6 | 164.0 | 1650 |
| 10. subject 2 serum 12/08 | <3 | pg/mL | 8.8 | 160.0 | 1750 |
| 11. Human IgG | 3 | pg/mL | 7.8 | 24.4 | 21.0 |
| 12. Swiss | 3.9 | pg/mL | 7.8 | 31.2 | 31.2 |
| 13. Human-Indonesia | 3 | pg/mL | 8.8 | 568 | 2200 |

Since modifications will be apparent to those of skill in this art, it is intended that this invention be limited only by the scope of the appended claims.

What is claimed is:

1. A method of treating a disease, condition or disorder, comprising:
administering blood or a soluble-receptor containing fraction thereof to a mammal suffering from an acute inflammatory condition, wherein prior to administration the blood or fraction thereof has been contacted with tetracycline or a tetracycline-like compound, whereby the level of a pre-selected cytokine receptor in the blood is at least three-fold higher than level of the receptors prior to contacting with the tetracycline or tetracycline-like compound.

2. The method of claim 1, wherein the cytokine receptor is a tumor necrosis factor (TNF) receptor and/or an interleukin-1 receptor (IL-1R).

3. The method of claim 1, wherein the disease, condition or disorder is selected from the group consisting of acute inflammatory conditions associated with viral hemorrhagic diseases, parasitic diseases, bacterial infections, sepsis, cachexia, autoimmune disorders, acute cardiovascular events, chronic myelogenous leukemia and transplanted bone marrow-induced graft-versus-host disease, septic shock, immune complex-induced colitis, cerebrospinal fluid inflammation, autoimmune disorders, multiple sclerosis; inflammatory responses associated with trauma; systemic inflammatory response syndrome (SIRS), adult respiratory distress syndrome (ARDS), acute liver failure, inflammatory bowel disease and Crohn's disease.

4. The method of claim 1, wherein the disease, condition or disorder is selected from viral hemorrhagic diseases and bacterial infections.

5. The method of claim 1, wherein the contacting with tetracycline or a tetracycline-like compound is effected by administering the tetracycline or tetracycline-like compound to the donor of the blood.

6. The method of claim 1, wherein the mammal has a viral hemorrhagic disease.

7. A method for treating or preventing a viral hemorrhagic disease, comprising administering an effective amount of a tetracycline or tetracycline-like compound, whereby the viral hemorrhagic disease is treated or prevented.

8. The method of claim 7, further comprising administering a blood-derived composition, wherein:
the composition is produced by
i) obtaining blood from a mammalian donor and measuring the level of a cytokine antagonist or cytokine receptor in the blood; and
ii) administering to the mammalian donor or contacting blood from the donor with a tetracycline or tetracycline-like compound(s) in an amount sufficient and for a time sufficient to result in a three-fold increase in the measured cytokine antagonist or receptor; and
the composition is administered simultaneously, subsequently or before administration of the tetracycline or tetracycline-like compound.

9. The method of claim 7, further comprising administering an anti-hemorrhagic viral treatment or agent to the mammal.

10. A combination, comprising:
a) a tetracycline compound; and
b) an anti-hemorrhagic virus treatment or agent.

11. The combination of claim 10, wherein the tetracycline compound and the anti-hemorrhagic virus agent are formulated in a single pharmaceutical composition or each formulated in a separate pharmaceutical compositions.

12. The combination of claim 10, wherein the tetracycline compound is selected from the group consisting of chlortetracycline, demeclocycline, doxycycline, methacycline, minocycline, oxytetracycline and tetracycline.

13. The combination of claim 10, wherein the hemorrhagic virus is a Bunyaviridae, a Filoviridae, a Flaviviridae, or an Arenaviridae virus.

14. The combination of claim 10, wherein the anti-hemorrhagic virus agent inhibits interleukin-1 (IL-1) and/or tumor necrosis factor (TNF).

15. The combination of claim 14, wherein the agent that inhibits IL-1 is selected from the group consisting of anti-IL-1 antibodies, anti-IL-1 receptor antibodies, IL-1 receptor antagonists, IL-1 production inhibitors, IL-1 receptor production inhibitors, and IL-1 releasing inhibitors.

16. The combination of claim 14, wherein the TNF inhibitor is selected from the group consisting of an anti-TNF antibody, an anti-TNF receptor antibody, a TNF receptor antagonist, a TNF production inhibitor, a TNF receptor production inhibitor and a TNF releasing inhibitor.

17. The combination of claim 10, wherein the anti-viral-hemorrhagic agent is selected from the group consisting of an anti-viral vaccine, an anti-viral antibody, a viral-activated immune cell and a viral-activated immune serum.

18. The method of claim 9, wherein the mammal is a human.

19. The method of claim 18, wherein the tetracycline compound is selected from the group consisting of chlortetracycline, demeclocycline, doxycycline, methacycline, minocycline, oxytetracycline and tetracycline.

20. The method of claim 9, wherein the anti-viral hemorrhagic treatment comprises administering an effective amount of an anti-viral-hemorrhagic agent.

21. The method of claim 20, wherein the tetracycline compound and the anti-viral-hemorrhagic agent are administered sequentially.

22. The method of claim 21, wherein the tetracycline compound and the anti hemorrhagic virus treatment are co-administered.

23. The method of claim 22, wherein the tetracycline compound and the anti-viral-hemorrhagic agent are administered in the same composition.

24. The method of claim 9, wherein the anti-hemorrhagic virus agent inhibits interleukin-1 (IL-1) and/or tumor necrosis factor (TNF).

25. The method of claim 24, wherein the agent that inhibits IL-1 is selected from the group consisting of anti-IL-1 antibodies, anti-IL-1 receptor antibodies, IL-1 receptor antagonists, IL-1 production inhibitors, IL-1 receptor production inhibitors, and IL-1 releasing inhibitors.

26. The method of claim 25, wherein the TNF inhibitor is selected from the group consisting of an anti-TNF antibody, an anti-TNF receptor antibody, a TNF receptor antagonist, a TNF production inhibitor, a TNF receptor production inhibitor and a TNF releasing inhibitor.

27. A kit, comprising the combination of claim 10 and instructions for administration of the components for treatment of a hemorrhagic viral infection.

28. An article of manufacture, comprising:
packaging material;
a tetracycline compound or a tetracycline-like compound(s) in an amount effective for treating a hemorrhagic viral infection; and
a label indicating that the tetracycline compound is for use in treating a hemorrhagic viral infection.

29. A method for producing a cytokine-receptor-enriched blood product, comprising:
treating blood or a fraction thereof with a tetracycline or tetracycline-like compound; and
harvesting the plasma, wherein the plasma is enriched for cytokine receptors compared to the blood prior to treatment.

30. The method of claim 29, wherein the receptors are soluble tumor necrosis factor (TNF) receptors and/or interleukin-1 (IL-1) receptors.

31. The method of claim 29, wherein the blood is contacted in vitro.

32. The method of claim 29, wherein the blood is contacted in vivo.

33. The method of claim 29, further comprising harvesting the globulin fraction.

34. A method for producing cytokine-receptor-enriched compositions, comprising:
treating white blood cells in vitro with a tetracycline or tetracycline-like compound, whereby receptor expression is induced; and
collecting extracellular medium.

35. The method of claim 34, further comprising:
fractionating the medium to collect fraction(s) that contain the receptors.

36. The method of claim 34, wherein the receptors comprise soluble tumor necrosis factor (TNF) receptors and/or interleukin-1 (IL-1) receptors.

37. The method of claim 34, further comprising isolating IL-1 and/or TNF receptors therefrom.

38. A soluble receptor-containing composition produced by the method of claim 29.

39. A soluble receptor-containing composition produced by the method of claim 34.

40. A method of treatment of a mammal having an acute inflammatory condition, disease or disorder, comprising administering the composition of claim 59.

41. The method of claim 40, wherein the acute inflammatory condition is selected from the group consisting of acute inflammatory conditions associated with viral hemorrhagic diseases, parasitic diseases, bacterial infections, sepsis, cachexia, autoimmune disorders, acute cardiovascular events, chronic myelogenous leukemia and transplanted bone marrow-induced graft-versus-host disease, septic shock, immune complex-induced colitis, cerebrospinal fluid inflammation, autoimmune disorders, multiple sclerosis; inflammatory responses associated with trauma; systemic inflammatory response syndrome (SIRS), adult respiratory distress syndrome (ARDS), acute liver failure, inflammatory bowel disease and Crohn's disease.

42. A method of treatment of a mammal having an acute inflammatory condition, disease or disorder, comprising administering the composition of claim 39.

43. The method of claim 42, wherein the acute inflammatory condition is selected from the group consisting of acute inflammatory conditions associated with viral hemorrhagic diseases, parasitic diseases, bacterial infections, sepsis, cachexia, autoimmune disorders, acute cardiovascular events, chronic myelogenous leukemia and transplanted bone marrow-induced graft-versus-host disease, septic shock, immune complex-induced colitis, cerebrospinal fluid inflammation, autoimmune disorders, multiple sclerosis; inflammatory responses associated with trauma; systemic inflammatory response syndrome (SIRS), adult respiratory distress syndrome (ARDS), acute liver failure, inflammatory bowel disease and Crohn's disease.

44. A method for treatment or prophylaxis of an inflammatory disease, comprising administering an effective amount of a tetracycline or tetracycline-like compound, whereby the disease is treated or prevented, and wherein the disease, condition or disorder is selected from the group consisting of multiple sclerosis, rheumatoid arthritis, and inflammatory responses associated with systemic inflammatory response syndrome (SIRS), adult respiratory distress syndrome (ARDS), acute liver failure, inflammatory bowel disease, polytrauma, burns, major surgery or Crohn's disease.

45. The method of claim 44, wherein the tetracycline compound is selected from the group consisting of chlortetracycline, demeclocycline, doxycycline, methacycline, minocycline, oxytetracycline and tetracycline.


SEQUENCE LISTING (26 sequences)

SEQ ID NO: 1 - protein, 271 aa, Homo sapiens
  Recombinant Interleukin 1-alpha
MAKVPDMFEDLKNCYSENEEDSSSIDHLSLNQKSFYHVSYGPLHEGCMDQSVSLSISETSKTSKLTFKESMVVVATNGKVLKKRRLSLSQSITDDDLEAIANDSEEEIIKPRSAPFSFLSNVKYNFMRIIKYEFILNDALNQSIIRANDQYLTAAALHNLDEAVKFDMGAYKSSKDDAKITVILRISKTQLYVTAQDEDQPVLLKEMPEIPKTITGSETNLLFFWETHGTKNYFTSVAHPNLFIATKQDYWVCLAGGPPSITDFQILENQA

SEQ ID NO: 2 - protein, 269 aa, Homo sapiens
  Interleukin-1 beta (catabolin)
MAEVPKLASEMMAYYSGNEDDLFFEADGPKQMKCSFQDLDLCPLDGGIQLRISDHHYSKGFRQAASVVVAMDKLRKMLVPCPQTFQENDLSTFFPFIFEEEPIFFDTWDNEAYVHDAPVRSLNCTLRDSQQKSLVMSGPYELKALHLQGQDMEQQVVFSMSFVQGEESNDKIPVALGLKEKNLYLSCVLKDDKPTLQLESVDPKNYPKKKMEKRFVFNKIEINNKLEFESAQFPNWYISTSQAENMPVFLGGTKGGQDITDFTMQFVSS

SEQ ID NO: 3 - protein, 569 aa, Homo sapiens
  Interleukin-1 receptor, Type I precursor
MKVLLRLICFIALLISSLEADKCKEREEKIILVSSANEIDVRPCPLNPNEHKGTITWYKDDSKTPVSTEQASRIHQHKEKLWFVPAKVEDSGHYYCVVRNSSYCLRIKISAKFVENEPNLCYNAQAIFKQKLPVAGDGGLVCPYMEFFKNENNELPKLQWYKDCKPLLLDNIHFSGVKDRLIVMNVAEKHRGNYTCHASYTYLGKQYPITRVIEFITLEENKPTRPVIVSPANETMEVDLGSQIQLICNVTGQLSDIAYWKWNGSVIDEDDPVLGEDYYSVENPANKRRSTLITVLNISEIESRFYKHPFTCFAKNTHGIDAAYIQLIYPVTNFQKHMIGICVTLTVIIVCSVFIYKIFKIDIVLWYRDSCYDFLPIKASDGKTYDAYILYPKTVGEGSTSDCDIFVFKVLPEVLEKQCGYKLFIYGRDDYVGEDIVEVINENVKKSRRLIIILVRETSGFSWLGGSSEEQIAMYNALVQDGIKVVLLELEKIQDYEKMPESIKFIKQKHGAIRWSGDFTQGPQSAKTRFWKNVRYHMPVQRRSPSSKHQLLSPATKEKLQREAHVPLG

SEQ ID NO: 4 - protein, 398 aa, Homo sapiens
  Interleukin-1 receptor, Type II precursor
MLRLYVLVMGVSAFTLQPAAHTGAARSCRFRGRHYKREFRLEGEPVALRCPQVPYWLWASVSPRINLTWHKNDSARTVPGEEETRMWAQDGALWLLPALQEDSGTYVCTTRNASYCDKMSIELRVFENTDAFLPFISYPQILTLSTSGVLVCPDLSEFTRDKTDVKIQWYKDSLLLDKDNEKFLSVRGTTHLLVHDVALEDAGYYRCVLTFAHEGQQYNITRSIELRIKKKKEETIPVIISPLKTISASLGSRLTIPCKVFLGTGTPLTTMLWWTANDTHIESAYPGGRVTEGPRQEYSENNENYIEVPLIFDPVTREDLHMDFKCVVHNTLSFQTLRTTVKEASSTFSWGIVLAPLSLAFLVLGGIWMHRRCKHRTGKADGLTVLWPHHQDFQSYPK

SEQ ID NO: 5 - protein, 177 aa, Homo sapiens
  Interleukin-1 Receptor Antagonist Protein Precursor (IL-1RA; ICIL-1RA; IRAP)
MEICRGLRSHLITLLLFLFHSETICRPSGRKSSKMQAFRIWDVNQKTFYLRNNQLVAGYLQGPNVNLEEKIDVVPIEPHALFLGIHGGKMCLSCVKSGDETRLQLEAVNITDLSENRKQDKRFAFIRSDSGPTTSFESAACPGWFLCTAMEADQPVSLTNMPDEGVMVTKFYFQEDE

SEQ ID NO: 6 - protein, 176 aa, Homo sapiens
  IL-1 receptor intracellular ligand protein comprising amino acid sequence
IPRVDLRVWQDCCEDCRTRGQFNAFSYHFRGRRSLEFSYQEDKPTKKTRPRKIPSVGRQGEHLSNSTSAFSTRSDASGTNDFREFVLEMQKTITDLRTQIKKLESRLSTTECVDAGGESHANNTKWKKDACTICECKDGQVTCFVEACPPATCAVPVNIPGACCPVCLQKRAEEKP

SEQ ID NO: 7 - protein, 320 aa, Homo sapiens
  IL-1 receptor intracellular ligand protein comprising amino acid sequence
KKGGKTEQDGYQKPTNKHFTQSPKKSVADLLGSFEGKRRLLLITAPKAENNMYVQQRDEYLESFCKMATRKISVITIFGPVNNSTMKIDHFQLDNEKPMRVVDDEDLVDQRLISELRKEYGMTYNDFFMVLTDVDLRVKQYYEVPITMKSVFDLIDTFQSRIKDMEKQKKEGIVCKEEVGGVLELFPINGSSVVEREDVPAHLVKDIRNYFQVSPEYFSMLLVGKDGNVKSWYPSPMWSMVIVYDLIDSMQLRRQEMAIQQSLGMRCQKMSMQAMVTIVTTKDTRMVTRMTTVIMRVITMDTLTEQKYVTLDSASFLCSC

SEQ ID NO: 8 - protein, 251 aa, Homo sapiens
  IL-1 receptor intracellular ligand protein comprising amino acid sequence
KNFFLTNRARERSDTFINLREVLNRFKLPPGEYILVPSTFEPNKDGDFCIRVFSEKKADYQAVDDEIEANLEEFDISEDDIDDGFRRLFAQLAGEDAEISAFELQTILRRVLAKRQDIKSDGFSIETCKIMVDMLDSDGSGKLGLKEFYILWTKIQKYQKIYREIDVDRSGTMNSYEMRKALEEAGFKMPCQLHQVIVARFADDQLIIDFDNFVRCLVRLETLFKIFKQLDPENTGTIELDLISWLCFSVL

SEQ ID NO: 9 - protein, 700 aa, Homo sapiens
  IL-1 receptor intracellular ligand protein comprising amino acid sequence
MAGIAAKLAKDREAAEGLGSHERAIKYLNQDYEALRNECLEAGTLFQDPSFPAIPSALGFKELGPYSSKTRGMRWKRPTEICADPQFIIGGATRTDICQGALGDCWLLAAIASLTLNEEILARVVPLNQSFQENYAGIFHFQFWQYGEWVEVVVDDRLPTKDGELLFVHSAEGSEFWSALLEKAYAKINGCYEALSGGATTEGFEDFTGGIAEWYELKKPPPNLFKIIQKALQKGSLLGCSIDITSAADSEAITFQKLVKGHAYSVTGAEEVESNGSLQKLIRIRNPWGEVEWTGRWNDNCPSWNTIDPEERERLTRRHEDGEFWMSFSDFLRHYSRLEICNLTPDTLTSDTYKKWKLTKMDGNWRRGSTAGGCRNYPNTFWMNPQYLIKLEEEDEDEEDGESGCTFLVGLIQKHRRRQRKMGEDMHTIGFGIYEVPEELSGQTNIHLSKNFFLTNRARERSDTFINLREVLNRFKLPPGEYILVPSTFEPNKDGDFCIRVFSEKKADYQAVDDEIEANLEEFDISEDDIDDGVRRLFAQLAGEDAEISAFELQTILRRVLAKRQDIKSDGFSIETCKIMVDMLDSDGSGKLGLKEFYILWTKIQKYQKIYREIDVDRSGTMNSYEMRKALEEAGFKMPCQLHQVIVARFADDQLIIDFDNFVRCLVRLETLFKIFKQLDPENTGTIELDLISWLCFSVL

SEQ ID NO: 10 - DNA, 18 bp, Artificial Sequence
  Description of Artificial Sequence Synthetic DNA which is antisense to human IL-1 beta
ctcaggtacttctgccat

SEQ ID NO: 11 - DNA, 24 bp, Artificial Sequence
  Description of Artificial Sequence Synthetic DNA which is antisense to human IL-1 alpha
tggatgggcaactgatgtgaaata

SEQ ID NO: 12 - DNA, 20 bp, Artificial Sequence
  Description of Artificial Sequence Synthetic phosphorothioate DNA which is antisense to IL-1 receptor
tgtgtcctgcaatcggtggc

SEQ ID NO: 13 - DNA, 18 bp, Artificial Sequence
  Description of Artificial Sequence Synthetic phosphodiester or phosphorothioate DNA which is antisense to human IL-1 receptor
tctgagtaacactttcat

SEQ ID NO: 14 - protein, 233 aa, Homo sapiens
  Tumor Necrosis Factor Precursor (TNF-alpha; Cachectin)
MSTESMIRDVELAEEALPKKTGGPQGSRRCLFLSLFSFLIVAGATTLFCLLHFGVIGPQREEFPRDLSLISPLAQAVRSSSRTPSDKPVAHVVANPQAEGQLQWLNRRANALLANGVELRDNQLVVPSEGLYLIYSQVLFKGQGCPSTHVLLTHTISRIAVSYQTKVNLLSAIKSPCQRETPEGAEAKPWYEPIYLGGVFQLEKGDRLSAEINRPDYLDFAESGQVYFGIIAL

SEQ ID NO: 15 - protein, 205 aa, Homo sapiens
  Tumor Necrosis Factor Beta (Lymphotoxin Alpha)
MTPPERLFLPRVCGTTLHLLLLGLLLVLLPGAQGLPGVGLTPSAAQTARQHPKMHLAHSTLKPAAHLIGDPSKQNSLLWRANTDRAFLQDGFSLSNNSLLVPTSGIYFVYSQVVFSGKAYSPKATSSPLYLAHEVQLFSSQYPFHVPLLSSQKMVYPGLQEPWLHSMYHGAAFQLTQGDQLSTHTDGIPHLVLSPSTVFFGAFAL

SEQ ID NO: 16 - protein, 455 aa, Homo sapiens
  Tumor Necrosis Factor p55 Receptor
MGLSTVPDLLLPLVLLELLVGIYPSGVIGLVPHLGDREKRDSVCPQGKYIHPQNNSICCTKCHKGTYLYNDCPGPGQDTDCRECESGSFTASENHLRHCLSCSKCRKEMGQVEISSCTVDRDTVCGCRKNQYRHYWSENLFQCFNCSLCLNGTVHLSCQEKQNTVCTCHAGFFLRENECVSCSNCKKSLECTKLCLPQIENVKGTEDSGTTVLLPLVIFFGLCLLSLLFIGLMYRYQRWKSKLYSIVCGKSTPEKEGELEGTTTKPLAPNPSFSPTPGFTPTLGFSPVPSSTFTSSSTYTPGDCPNFAAPRREVAPPYQGADPILATALASDPIPNPLQKWEDSAHKPQSLDTDDPATLYAVVENVPPLRWKEFVRRLGLSDHEIDRLELQNGRCLREAQYSMLATWRRRTPRREATLELLGRVLRDMDLLGCLEDIEEALCGPAALPPAPSLLR

SEQ ID NO: 17 - protein, 461 aa, Homo sapiens [sequence omitted]

SEQ ID NO: 18 - protein, 410 aa, Homo sapiens
  TNF receptor death domain ligand protein comprising amino acid sequence
SNAGDGPGGEGSVHLASSRGTLSDSEIETNSATSTIFGKAHSLKPSIKEKLAGSPIRTSEDVSQRVYLYEGLLGKERSTLWDQMQFWEDAFLDAVMLEREGMGMDQGPQEMIDRYLSLGEHDRKRLEDDEDRLLATLLHNLISYMLLMKVNKNDIRKKVRRLMGKSHIGLVYSQQINEVLDQLANLNGRDLSIWSSGSRHMKKQTFVVHAGTDTNGDIFFMEVCDDCVVLRSNIGTVYERWWYEKLINMTYCPKTKVLCLWRRNGSETQLNKFYTKKCRELYYCVKDSMERAAARQQSIKPGPELGGEFPVQDLKTGEGGLLQVTLEGINLKFMHNQVFIELNHIKKCNTVRGVFVLEEFVPEIKEVVSHKYKTPMAHEICYSVLCLFSYVAAVHSSEEDLRTPPRPVSS

SEQ ID NO: 19 - protein, 138 aa, Homo sapiens
  TNF receptor death domain ligand protein comprising amino acid sequence
EVQDLFEAQGNDRLKLLVLYSGEDDELLQRAAAGGLAMLTSMRPTLCSRIPQVTTHWLEILQALLLSSNQELQHRGAVVVLNMVEASREIASTLMESEMMEILSVLAKGDHSPVTRAAAACLDKAVEYGLIQPNQDGE

SEQ ID NO: 20 - protein, 310 aa, Homo sapiens
  TNF receptor death domain ligand protein comprising amino acid sequence
SLKANIPEVEAVLNTDRSLVCDGKRGLLTRLLQVMKKEPAESSFRFWQARAVESFLRGTTSYADQMFLLKRGLLEHILYCIVDSECKSRDVLQSYFDLLGELMKFNVDAFKRFNKYINTDAKFQVFLKQINSSLVDSNMLVRCVTLSLDRFENQVDMKVAEVLSECRLLAYISQVPTQMSFLFRLINIIHVQTLTQENVSCLNTSLVILMLARRKERLPLYLRLLQRMEHSKKYPGFLLNNFHNLLRFWQQHYLHKDKDSTCLENSSCISFSYWKETVSILLNPDRQSPSALVSYIEEPYMDIDRDFTEE

SEQ ID NO: 21 - protein, 607 aa, Homo sapiens
  TNF receptor death domain ligand protein comprising amino acid sequence
EISRKVYKGMLDLLKCTVLSLEQSYAHAGLGGMASIFGLLEIAQTHYYSKEPDKRKRSPTESVNTPVGKDPGLAGRGDPKAMAQLRVPQLGPRAPSATGKGPKELDTRSLKEENFIASIGPEVIKPVFDLGETEEKKSQISADSGVSLTSSSQRTDQDSVIGVSPAVMIRSSSQDSEVSTVVSNSSGETLGADSDLSSNAGDGPGGEGSVHLASSRGTLSDSEIETNSATSTIFGKAHSLKPSIKEKLAGSPIRTSEDVSQRVYLYEGLLGKERSTLWDQMQFWEDAFLDAVMLEREGMGMDQGPQEMIDRYLSLGEHDRKRLEDDEDRLLATLLHNLISYMLLMKVNKNDIRKKVRRLMGKSHIGLVYSQQINEVLDQLANLNGRDLSIWSSGSRHMKKQTFVVHAGTDTNGDIFFMEVCDDCVVLRSNIGTVYERWWYEKLINMTYCPKTKVLCLWRRNGSETQLNKFYTKKCRELYYCVKDSMERAAARQQSIKPGPELGGEFPVQDLKTGEGGLLQVTLEGINLKFMHNQVFIELNHIKKCNTVRGVFVLEEFVPEIKEVVSHKYKTPMAHEICYSVLCLFSYVAAVHSSEEDLRTPPRPVSS

SEQ ID NO: 22 - DNA, 18 bp, Artificial Sequence
  Description of Artificial Sequence Synthetic DNA which is antisense to TNF-alpha
tcatggtgtcctttgcag

SEQ ID NO: 23 - DNA, 26 bp, Artificial Sequence
  Description of Artificial Sequence Synthetic DNA upper primer for Dengue virus type 2 detection
aatatgctgaaacgcgagagaaaccg

SEQ ID NO: 24 - DNA, 22 bp, Artificial Sequence
  Description of Artificial Sequence Synthetic DNA lower primer for Dengue virus type 2 detection
aaggaacgccaccaaggccatg

SEQ ID NO: 25 - DNA, 29 bp, Artificial Sequence
  Description of Artificial Sequence Synthetic DNA upper primer for IL-1ra detection
cgggatccgggagaaaatccagcaagatg

SEQ ID NO: 26 - DNA, 24 bp, Artificial Sequence
  Description of Artificial Sequence Synthetic DNA lower primer for IL-1ra detection
aggtcctgctcatccccttaaggc